(19) United States
(12) Patent Application Publication
(10) Pub. No.: US 2023/0257430 A1
(43) Pub. Date: Aug. 17, 2023

(54) RECOMBINANT PROTEINS FOR QUANTIFICATION OF PROTEIN LEVELS

(71) Applicant: Genentech, Inc., South San Francisco, CA (US)

(72) Inventors: Jason LIANG, Pacifica, CA (US); Avinashnarayan VENKATANARAYAN, Foster City, CA (US); Lai Man Phu YEE, Fremont, CA (US); Donald Scott KIRKPATRICK, San Mateo, CA (US)

(21) Appl. No.: 18/309,003

(22) Filed: Apr. 28, 2023

Related U.S. Application Data
(63) Continuation of application No. PCT/US2021/057429, filed on Oct. 29, 2021.
(60) Provisional application No. 63/108,009, filed on Oct. 30, 2020.

(51) Int. Cl. C07K 14/47; G01N 33/68; C12N 9/12

(52) U.S. Cl. CPC C07K 14/4702; G01N 33/6848; C12Y 207/12002; C12N 9/12; G01N 2458/15

(57) ABSTRACT

Provided herein are recombinant proteins comprising sets of polypeptides that may be used for measurement of protein levels.

### CROSS-REFERENCE TO RELATED APPLICATIONS

This application is a continuation of International Patent Application No. PCT/US2021/057429, filed on Oct. 29, 2021, which claims benefit of U.S. Provisional Application No. 63/108,009, filed Oct. 30, 2020, the contents of which are hereby incorporated by reference in their entirety.

### SEQUENCE LISTING

The instant application contains a Sequence Listing which has been submitted electronically in XML format and is hereby incorporated by reference in its entirety. Said XML copy, created on Apr. 11, 2023, is named 50474-230003_Sequence_Listing_4_11_23.xml and is 92,718 bytes in size.

### FIELD OF THE INVENTION

Provided herein are recombinant proteins comprising sets of polypeptides that may be used for quantification of protein levels.

### BACKGROUND

Perturbations in the MAPK and PI3K signaling pathways are involved in the development of many cancers. Thus, there is a need in the art for methods of quantifying protein levels of members of the MAPK and PI3K signaling pathways, as well as recombinant proteins for use in such methods.

### SUMMARY OF THE INVENTION

In one aspect, the disclosure features a recombinant protein comprising a set of non-identical, contiguous polypeptides, the set comprising: a polypeptide consisting of a sequence present in RAF1; a polypeptide consisting of a sequence present in BRAF; a polypeptide consisting of a sequence present in BRAF^{V600E}; a polypeptide consisting of a sequence present in ARAF; a polypeptide consisting of a sequence present in MP2K1; a polypeptide consisting of a sequence present in MP2K2; a polypeptide consisting of a sequence present in MK03; a polypeptide consisting of a sequence present in MK01; a polypeptide consisting of a sequence present in RASK; a polypeptide consisting of a sequence present in RASN; a polypeptide consisting of a sequence present in RASH; a polypeptide consisting of a sequence present in each of RAF1, BRAF, and ARAF; a polypeptide consisting of a sequence present in both of RASH and RASN; a polypeptide consisting of a sequence present in both of RASN and RASK; a polypeptide consisting of a sequence present in each of RASH, RASN, and RASK; a polypeptide consisting of a sequence present in each of RASH^{Q61K}, RASN^{Q61K}, and RASK^{Q61K}; a polypeptide consisting of a sequence present in each of RASH^{Q61R}, RASN^{Q61R}, and RASK^{Q61R}; a polypeptide consisting of a sequence present in each of RASH^{G12V}, RASN^{G12V}, and RASK^{G12V}; a polypeptide consisting of a sequence present in each of RASH^{G13D}, RASN^{G13D}, and RASK^{G13D}; a polypeptide consisting of a sequence present in each of RASH^{G12C}, RASN^{G12C}, and RASK^{G12C}; a polypeptide consisting of a sequence present in each of RASH^{G12D}, RASN^{G12D}, and RASK^{G12D}; and a polypeptide consisting of a sequence present in each of RASH^{G12S}, RASN^{G12S}, and RASK^{G12S}; wherein the recombinant protein comprises a trypsin cleavage site between each polypeptide of the set that allows separation of each polypeptide upon exposure of the recombinant protein to trypsin.

In some aspects, each of the polypeptides is between 6 and 25 amino acid residues in length.

In some aspects, the polypeptide consisting of a sequence present in BRAF^{V600E} has the amino acid sequence of SEQ ID NO: 10; the polypeptide consisting of a sequence present in each of RASH^{Q61K}, RASN^{Q61K}, and RASK^{Q61K} has the amino acid sequence of SEQ ID NO: 37; the polypeptide consisting of a sequence present in each of RASH^{Q61R}, RASN^{Q61R}, and RASK^{Q61R} has the amino acid sequence of SEQ ID NO: 38; the polypeptide consisting of a sequence present in each of RASH^{G12V}, RASN^{G12V}, and RASK^{G12V} has the amino acid sequence of SEQ ID NO: 39; the polypeptide consisting of a sequence present in each of RASH^{G13D}, RASN^{G13D}, and RASK^{G13D} has the amino acid sequence of SEQ ID NO: 40; the polypeptide consisting of a sequence present in each of RASH^{G12C}, RASN^{G12C}, and RASK^{G12C} has the amino acid sequence of SEQ ID NO: 41; the polypeptide consisting of a sequence present in each of RASH^{G12D}, RASN^{G12D}, and RASK^{G12D} has the amino acid sequence of SEQ ID NO: 42; and/or the polypeptide consisting of a sequence present in each of RASH^{G12S}, RASN^{G12S}, and RASK^{G12S} has the amino acid sequence of SEQ ID NO: 43.

In some aspects, the set comprises at least two polypeptides consisting of a sequence present in RAF1, BRAF, ARAF, MP2K1, MP2K2, MK03, MK01, RASK, RASN, or RASH; at least two polypeptides consisting of a sequence present in both of RASH and RASN; and/or at least two polypeptides consisting of a sequence present in each of RASH, RASN, and RASK.

In some aspects, the set further comprises one or more polypeptides consisting of a sequence present in one or more additional target molecules, and wherein the recombinant protein comprises a trypsin cleavage site between each of the one or more polypeptides that allows separation of each polypeptide upon exposure of the recombinant protein to trypsin.

In another aspect, the disclosure features a recombinant protein comprising a set of polypeptides having the amino acid sequences of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, and SEQ ID NO: 43.

In some aspects, the recombinant protein further comprises an N-terminal sequence comprising methionine and a trypsin cleavage site between the N-terminal sequence and the set of polypeptides that allows separation of the N-terminal sequence from the set of polypeptides upon exposure of the recombinant protein to trypsin. In some aspects, the N-terminal sequence has the amino acid sequence of SEQ ID NO: 88.

In some aspects, the recombinant protein further comprises a C-terminal sequence comprising a tag and a trypsin cleavage site between the C-terminal sequence and the set of polypeptides that allows separation of the C-terminal sequence from the set of polypeptides upon exposure of the recombinant protein to trypsin. In some aspects, the tag is a polyhistidine tag. In some aspects, the C-terminal sequence has the amino acid sequence of SEQ ID NO: 89. In some aspects, the tag is a FLAG tag or a V5 tag.

In some aspects, the recombinant protein comprises the amino acid sequence of SEQ ID NO: 1.

In another aspect, the disclosure features a recombinant protein consisting of the amino acid sequence of SEQ ID NO: 1.

In some aspects, each polypeptide of the set comprises a label. In some aspects, the label is an isotopic label. In some aspects, the isotopic label is heavy arginine. In some aspects, the heavy arginine is ¹³C₁-arginine (R1); ¹³C₂-arginine (R2); ¹⁵N₄-arginine (R4); ¹³C₆-arginine (R6); ²H₇-arginine (R7); ¹³C₆, ¹⁵Ne₄-arginine (R10); ²H₇, ¹⁵N₄-arginine (R11), or ¹³C₆, ²H₇, ¹⁵N₄-arginine (R17). In some aspects, the isotopic label is heavy lysine. In some aspects, the heavy lysine is ¹³C lysine (K1); ¹⁵N₂-lysine (K2); ²H₄-lysine (K4); ¹³C₆-lysine (K6); ¹³C₆, ¹⁵N₂-lysine (K8); ²H₉-lysine (K9); ²H₉-lysine (K9); ²H₉, ¹⁵N₂-lysine (K11); or ¹³C₆; ²H₉, ¹⁵N₂-lysine (K17). In some aspects, the label is a chemical label. In some aspects, the chemical label is a tandem mass tag (TMT), an iTRAQ, a label produced by reductive methylation/dimethylation, or a label produced by acetylation. In some aspects, the recombinant protein is at least 98% labeled. In some aspects, the recombinant protein is at least 99% labeled.

In another aspect, the disclosure features a method for determining a protein level in a sample from a subject of one or more of RAF1, BRAF, BRAF^{V600E}, ARAF, MP2K1, MP2K2, MK03, MK01, RASK, RASN, RASH; RASH and RASN; RASN and RASK; RASH, RASN, and RASK; RASH^{Q61K}, RASN^{Q61K} and RASK^{Q61K}; RASH^{Q61R}, RASN^{Q61R}, and RASK^{Q61R}; RASH^{G12V}, RASN^{G12V}, and RASK^{G12V}; RASH^{G13D}, RASN^{G13D}, and RASK^{G13D}; RASH^{G12C}, RASN^{G12C}, and RASK^{G12C}; RASH^{G12D}, RASN^{G12D}, and RASK^{G12D}; and RASH^{G12S}, RASN^{G12S}, and RASK^{G12S}; the method comprising: (a) adding to the sample an amount of a recombinant protein of the disclosure; (b) exposing the sample following step (a) to trypsin, whereby the recombinant protein is cleaved, thereby generating an equimolar set of internal standard polypeptides, the set comprising: a polypeptide consisting of a sequence present in RAF1; a polypeptide consisting of a sequence present in BRAF; a polypeptide consisting of a sequence present in BRAF^{V600E}; a polypeptide consisting of a sequence present in ARAF; a polypeptide consisting of a sequence present in MP2K1; a polypeptide consisting of a sequence present in MP2K2; a polypeptide consisting of a sequence present in MK03; a polypeptide consisting of a sequence present in MK01; a polypeptide consisting of a sequence present in RASK; a polypeptide consisting of a sequence present in RASN; a polypeptide consisting of a sequence present in RASH; a polypeptide consisting of a sequence present in each of RAF1, BRAF, and ARAF; a polypeptide consisting of a sequence present in both of RASH and RASN; a polypeptide consisting of a sequence present in both of RASN and RASK; a polypeptide consisting of a sequence present in each of RASH, RASN, and RASK; a polypeptide consisting of a sequence present in each of RASH^{Q61K}, RASN^{Q61K}, and RASK^{Q61K}; a polypeptide consisting of a sequence present in each of RASH^{Q61R}, RASN^{Q61R}, and RASK^{Q61R}; a polypeptide consisting of a sequence present in each of RASH^{G12V}, RASN^{G12V}, and RASK^{G12V}; a polypeptide consisting of a sequence present in each of RASH^{G13D}, RASN^{G13D}, and RASK^{G13D}; a polypeptide consisting of a sequence present in each of RASH^{G12C}, RASN^{G12C}, and RASK^{G12C}; a polypeptide consisting of a sequence present in each of RASH^{G12D}, RASN^{G12D}, and RASK^{G12D}; and a polypeptide consisting of a sequence present in each of RASH^{G12S}, RASN^{G12S}, and RASK^{G12S}; (c) measuring a level of one or more internal standard polypeptides and a level of one or more corresponding polypeptides from the sample; and (d) comparing the level of the one or more internal standard polypeptides and the level of the one or more corresponding polypeptides from the sample, thereby determining a protein level of one or more of: RAF1, BRAF, BRAF^{V600E}, ARAF, MP2K1, MP2K2, MK03, MK01, RASK, RASN, RASH; RASH and RASN; RASN and RASK; RASH, RASN, and RASK; RASH^{Q61K}, RASN^{Q61K}, and RASK^{Q61K}; RASH^{Q61R}, RASN^{Q61R}, and RASK^{Q61R}; RASH^{G12V}, RASN^{G12V}, and RASK^{G12V}; RASH^{G13D}, RASN^{G13D}, and RASK^{G13D}; RASH^{G12C}, RASN^{G12C}, and RASK^{G12C}; RASH^{G12D}, RASN^{G12D}, and RASK^{G12D}; and RASH^{G12S}, RASN^{G12S}, and RASK^{G12S} in the sample.

In some aspects, the method comprises determining a protein level of one or more of RASH, RASN, RASK, ARAF, BRAF, and RAF1 in the sample. In some aspects, the method comprises determining a protein level of each of RASH, RASN, RASK, ARAF, BRAF, and RAF1 in the sample.

In some aspects, the protein level is a relative protein level. In some aspects, the protein level is an absolute protein level.

In some aspects, the method is performed for at least two samples from the subject. In some aspects, the at least two samples are from at least two different time points. In some aspects, the at least two different time points include a time point before administration of an agent to the subject and a timepoint after administration of the agent to the subject.

In some aspects, the measuring of step (c) comprises mass spectrometry (MS). In some aspects, the MS is parallel reaction monitoring MS (PRM-MS).

In some aspects, the sample is a human sample. In some aspects, the sample is a tumor sample. In some aspects, the sample is a lysate. In some aspects, the sample is an immunoprecipitate of a target protein.

In some aspects, the method comprises determining the ratio of the target protein to one or more of RAF1, BRAF, BRAF^{V600E}, ARAF, MP2K1, MP2K2, MK03, MK01, RASK, RASN, RASH; RASH and RASN; RASN and RASK; RASH, RASN, and RASK; RASH^{Q61K}, RASN^{Q61K}, and RASK^{Q61K}; RASH^{Q61R}, RASN^{Q61R}, and RASK^{Q61R}; RASH^{G12V}, RASN^{G12V}, and RASK^{G12V}; RASH^{G13D}, RASN^{G13D}, and RASK^{G13D}; RASH^{G12C}, RASN^{G12C}, and RASK^{G12C}; RASH^{G12D}, RASN^{G12D}, and RASK^{G12D}; and RASH^{G12S}, RASN^{G12S}, and RASK^{G12S}.

In another aspect, the disclosure features a nucleic acid encoding a recombinant protein of the disclosure.

In another aspect, the disclosure features a recombinant protein comprising a set of non-identical, contiguous polypeptides, the set comprising: a polypeptide consisting of a sequence present in P85A; a polypeptide consisting of a sequence present in P85B; a polypeptide consisting of a sequence present in PK3CA; a polypeptide consisting of a sequence present in PK3CA^{E545K}; a polypeptide consisting of a sequence present in PK3CA^{H1047K}; a polypeptide consisting of a sequence present in PK3CD; a polypeptide consisting of a sequence present in PK3CB; a polypeptide consisting of a sequence present in ERBB2; a polypeptide consisting of a sequence present in EGFR; a polypeptide consisting of a sequence present in RRAS2; and a polypeptide consisting of a sequence present in P55G, wherein the recombinant protein comprises a trypsin cleavage site between each polypeptide of the set that allows separation of each polypeptide upon exposure of the recombinant protein to trypsin.

In some aspects, each of the polypeptides is between 6 and 25 amino acid residues in length.

In some aspects, the polypeptide consisting of a sequence present in PK3CA^{E545K} has the amino acid sequence of SEQ ID NO: 56 and/or the polypeptide consisting of a sequence present in PK3CA^{H1047K} has the amino acid sequence of SEQ ID NO: 58 or SEQ ID NO: 59.

In some aspects, the set comprises at least two polypeptides consisting of a sequence present in P85A, P85B, PK3CA, PK3CA^{E545K}, PK3CA^{H1047K}, PK3CD, PK3CB, ERBB2, EGFR, RRAS2, or P55G.

In some aspects, the recombinant protein further comprises a polypeptide consisting of a sequence present in a control protein. In some aspects, the control protein is G3P or ACTA. In some aspects, the recombinant protein comprises a polypeptide consisting of a sequence present in G3P and a polypeptide consisting of a sequence present in ACTA. In some aspects, the set comprises at least two polypeptides consisting of a sequence present in G3P or ACTA.

In some aspects, the recombinant protein further comprises one or more additional non-identical, contiguous polypeptides consisting of a sequence present in one or more additional target molecules, wherein each of the one or more additional polypeptides comprises a cleavage site that allows separation of the polypeptide from the set upon exposure of the recombinant protein to trypsin.

In another aspect, the disclosure features a recombinant protein comprising a set of polypeptides having the amino acid sequences of SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 82, SEQ ID NO: 83, SEQ ID NO: 84, SEQ ID NO: 85, SEQ ID NO: 86, and SEQ ID NO: 87.

In some aspects, the set of polypeptides further comprises polypeptides having the amino acid sequences of SEQ ID NO: 76, SEQ ID NO: 77, SEQ ID NO: 78, SEQ ID NO: 79, SEQ ID NO: 80, and SEQ ID NO: 81.

In some aspects, the recombinant protein further comprises an N-terminal sequence comprising methionine and a trypsin cleavage site between the N-terminal sequence and the set of polypeptides that allows separation of the N-terminal sequence from the set of polypeptides upon exposure of the recombinant protein to trypsin. In some aspects, the N-terminal sequence has the amino acid sequence of SEQ ID NO: 88.

In some aspects, the recombinant protein further comprises a C-terminal sequence comprising a tag and a trypsin cleavage site between the C-terminal sequence and the set of polypeptides that allows separation of the C-terminal sequence from the set of polypeptides upon exposure of the recombinant protein to trypsin. In some aspects, the tag is a polyhistidine tag. In some aspects, the C-terminal sequence has the amino acid sequence of SEQ ID NO: 89. In some aspects, the tag is a FLAG tag or a V5 tag. In some aspects, the recombinant protein comprises the amino acid sequence of SEQ ID NO: 44.

In another aspect, the disclosure features a recombinant protein consisting of the amino acid sequence of SEQ ID NO: 44.

In some aspects, each polypeptide of the set comprises a label. In some aspects, the label is an isotopic label. In some aspects, the isotopic label is heavy arginine. In some aspects, the heavy arginine is ¹³C₁-arginine (R1); ¹³C₂-arginine (R2); ¹⁵N₄-arginine (R4); ¹³C₆-arginine (R6); ²H₇-arginine (R7); ¹³C₆, ¹⁵N₄-arginine (R10); ²H₇, ¹⁵N₄-arginine (R11), or ¹³C₆, ²H₇, ¹⁵N₄-arginine (R17). In some aspects, the isotopic label is heavy lysine. In some aspects, the heavy lysine is ¹³C₁-lysine (K1); ¹⁵N₂-lysine (K2); ²H₄-lysine (K4); ¹³C₆-lysine (K6); ¹³C₆, ¹⁵N₂-lysine (K8); ²H₈-lysine (K8); ²H₉-lysine (K9); ²H₉, ¹⁵N₂-lysine (K11); or ¹³C₆; ²H₉, ¹⁵N₂-lysine (K17). In some aspects, the label is a chemical label. In some aspects, the chemical label is a tandem mass tag (TMT), an iTRAQ, a label produced by reductive methylation/dimethylation, or a label produced by acetylation.

In some aspects, the recombinant protein is at least 98% labeled. In some aspects, the recombinant protein is at least 99% labeled.

In another aspect, the disclosure features a method for determining a protein level in a sample from a subject of one or more of P85A, P85B, PK3CA, PK3CA^{E545K}, PK3CA^{H1047K}, PK3CD, PK3CB, ERBB2, EGFR, RRAS2, and P55G; the method comprising: (a) adding to the sample an amount of a recombinant protein of the disclosure; (b) exposing the sample following step (a) to trypsin, whereby the recombinant protein is cleaved, thereby generating an equimolar set of internal standard polypeptides, the set comprising: a polypeptide consisting of a sequence present in P85A; a polypeptide consisting of a sequence present in P85B; a polypeptide consisting of a sequence present in PK3CA; a polypeptide consisting of a sequence present in PK3CA^{E545K}; a polypeptide consisting of a sequence present in PK3CA^{H1047K}; a polypeptide consisting of a sequence present in PK3CD; a polypeptide consisting of a sequence present in PK3CB; a polypeptide consisting of a sequence present in ERBB2; a polypeptide consisting of a sequence present in EGFR; a polypeptide consisting of a sequence present in RRAS2; and a polypeptide consisting of a sequence present in P55G; (c) measuring a level of one or more internal standard polypeptides and a level of one or more corresponding polypeptides from the sample; and (d) comparing the level of the one or more internal standard polypeptides and the level of the one or more corresponding polypeptides from the sample, thereby determining a protein level of one or more of P85A, P85B, PK3CA, PK3CA^{E545K}, PK3CA^{H1047K}, PK3CD, PK3CB, ERBB2, EGFR, RRAS2, and P55G in the sample.

In some aspects, the method further comprises determining a protein level of G3P and/or ACTA in the sample from the subject, wherein the set of internal standard polypeptides of step (b) comprises a polypeptide consisting of a sequence present in G3P and/or a polypeptide consisting of a sequence present in ACTA.

In some aspects, the protein level is a relative protein level. In some aspects, the protein level is an absolute protein level.

In some aspects, the method is performed for at least two samples from the subject. In some aspects, the at least two samples are from at least two different time points. In some aspects, the at least two different time points include a time point before administration of an agent to the subject and a timepoint after administration of the agent to the subject.

In some aspects, the measuring of step (c) comprises mass spectrometry (MS). In some aspects, the MS is parallel reaction monitoring MS (PRM-MS).

In some aspects, the sample is a human sample. In some aspects, the sample is a tumor sample. In some aspects, the sample is a lysate. In some aspects, the sample is an immunoprecipitate of a target protein.

In some aspects, the method comprises determining the ratio of the target protein to one or more of P85A, P85B, PK3CA, PK3CA^{E545K}, PK3CA^{H1047K}, PK3CD, PK3CB, ERBB2, EGFR, G3P, ACTA, RRAS2, and P55G In another aspect, the disclosure features a nucleic acid encoding the recombinant protein of the disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a set of graphs showing the CERES scores of the RAF family members ARAF (left graph), BRAF (center graph), and RAF1 (CRAF) (right graph), in human cancer cells including lung, pancreatic, colon, skin, ovary, and breast cancer cell lines. Cancer cell lines were categorized based on the presence of BRAF^{V600E}, RASK* (KRAS*) or RASN* (NRAS*) mutations. Asterisk denotes that cell lines carry a mutation in RASK or RASN. Cell lines that do not carry these mutations are grouped as “other”. Student's t-test: asterisk indicates a significance of <0.005. ns: not significant.

FIG. 1B is a graph showing the CERES score of RAF1 (CRAF) in RASK (KRAS), RASN (NRAS), and RAF1 (CRAF) mutant cancer cell lines from the Achilles dataset.

FIG. 1C is a set of photomicrographs showing a Western blot analysis of the MAPK pathway components RAF1 (CRAF), pMEK (antibody detects MEK1 phosphorylated at Ser 217 and MEK2 phosphorylated at Ser 222), MEK (antibody detects MEK1 and MEK2), pERK (antibody detects ERK1 phosphorylated at Thr 202 and ERK2 phosphorylated at Tyr 204), and ERK (antibody detects ERK1 and ERK2) and a β actin control in the cancer cell lines A549, CALU6, TCC.PAN2, SW620, and HCT116 treated with a shRNA that depletes RAF1 (CRAF) (shCRAF) or a non-targeting shRNA (shNT). GDC-0973 (+) indicates that cells were co-treated with the MEK inhibitor GDC-0973 (24 hours, 250 nM). DOX (+) indicates that cells were treated with 0.5 μg/mL doxycycline (DOX) for 72 hours to induce expression of the shRNA. shCRAF-1 and shCRAF-2 are shRNA constructs targeting different regions of RAF1 (CRAF).

FIG. 1D is a pair of photomicrographs and a graph showing the results of a soft agar colony formation growth assay for the cancer cell line A549 treated with shCRAF. Colonies were stained with MTT dye and imaged using the GelCount software (n=5). The number (#) of colonies counted is reported. Cells not treated with DOX (“Without DOX”) are provided as a control.

FIG. 1E is a pair of photomicrographs and a graph showing the results of a soft agar colony formation growth assay for the cancer cell line CALU6 treated with shCRAF. Colonies were stained with MTT dye and imaged using the GelCount software (n=5). The number (#) of colonies counted is reported. Cells not treated with DOX (“Without DOX”) are provided as a control.

FIG. 1F is a pair of photomicrographs and a graph showing the results of a soft agar colony formation growth assay for the cancer cell line TCC.PAN2 treated with shCRAF. Colonies were stained with MTT dye and imaged using the GelCount software (n=5). The number (#) of colonies counted is reported. Cells not treated with DOX (“Without DOX”) are provided as a control.

FIG. 1G is a pair of photomicrographs and a graph showing the results of a soft agar colony formation growth assay for the cancer cell line SW620 treated with shCRAF. Colonies were stained with MTT dye and imaged using the GelCount software (n=5). The number (#) of colonies counted is reported. Cells not treated with DOX (“Without DOX”) are provided as a control.

FIG. 1H is a pair of photomicrographs and a graph showing the results of a soft agar colony formation growth assay for the cancer cell line HCT116 treated with shCRAF. Colonies were stained with MTT dye and imaged using the GelCount software (n=5). The number (#) of colonies counted is reported. Cells not treated with DOX (“Without DOX”) are provided as a control.

FIG. 1I is a set of photomicrographs showing the results of a soft agar colony formation growth assay for the cancer cell line A549 treated with an shRNA that depletes ARAF (shARAF), BRAF (shBRAF) and RAF1 (CRAF) (shCRAF) or a non-targeting shRNA (shNT). Colonies were stained with MTT dye and imaged using the GelCount software (n=5).

FIG. 1J is a heat map showing levels of DUSP6 mRNA, as measured using qRT-PCR, in the cancer cell lines A549, CALU6, TCC.PAN2, SW620, and HCT116 following treatment with shCRAF or a non-targeting shRNA (shNT). GDC-0973 (+) indicates that cells were co-treated with the MEK inhibitor GDC-0973 (24 hours, 250 nM). DOX (+) indicates that cells were treated with 0.5 μg/mL doxycycline (DOX) for 72 hours.

FIG. 1K is a heat map showing levels of SPRTY mRNA, as measured using qRT-PCR, in the cancer cell lines A549, CALU6, TCC.PAN2, SW620, and HCT116 following treatment with shCRAF or a non-targeting shRNA (shNT). GDC-0973 (+) indicates that cells were co-treated with the MEK inhibitor GDC-0973 (24 hours, 250 nM). DOX (+) indicates that cells were treated with 0.5 μg/mL doxycycline (DOX) for 72 hours.

FIG. 1L is a graph showing in vivo xenograft tumor growth inhibition curves in A549 cells treated with shCRAF. “With DOX” indicates that samples were treated with 0.5 mg/ml doxycycline (DOX). Samples not treated with DOX (“Without DOX”) are provided as a control. n=10 mice per genotype.

FIG. 1M is a graph showing in vivo xenograft tumor growth inhibition curves in CALU6 cells treated with shCRAF. “With DOX” indicates that cells were treated with 0.5 mg/mL doxycycline (DOX) hours. Samples not treated with DOX (“Without DOX”) are provided as a control. n=10 mice per genotype.

FIG. 1N is a graph showing in vivo xenograft tumor growth inhibition curves in SW620 cells treated with shCRAF. “With DOX” indicates that cells were treated with 0.5 mg/mL doxycycline (DOX) hours. Samples not treated with DOX (“Without DOX”) are provided as a control. n=10 mice per genotype.

FIG. 2A is a set of photomicrographs showing the results of a soft agar colony formation growth assay for the cancer cell lines SW48 and H₁₉₇₅ treated with an shRNA that depletes RAF1 (CRAF) (shCRAF). Colonies were stained with MTT dye and imaged using the GelCount software (n=5). “With DOX” indicates that cells were treated with 0.5 μg/mL doxycycline (DOX). Cells not treated with DOX (“Without DOX”) are provided as a control.

FIG. 2B is a set of photomicrographs showing a Western blot analysis of the MAPK pathway components ARAF, BRAF, RAF1 (CRAF), pMEK, MEK, pERK, ERK, pRSK, RSK, and a β actin control in cancer cell lines treated with shARAF, shBRAF, shCRAF, or a non-targeting shRNA (shNT). GDC-0973 (+) indicates that cells were co-treated with the MEK inhibitor GDC-0973 (24 hours, 250 nM). DOX (+) indicates that cells were treated with 0.5 μg/mL doxycycline (DOX) for 72 hours.

FIG. 2C is a graph showing the results of a short-term growth assay (percent confluence) in A549 cells treated with shCRAF or a shNT control (mean±SEM, n=5). “With DOX” indicates that cells were treated with doxycycline (DOX). Cells not treated with DOX (“Without DOX”) are provided as a control.

FIG. 2D is a set of photomicrographs showing the results of a long-term colony formation assay in A549 cells treated with shARAF, shBRAF, or shCRAF or a shNT control. Cells were stained with crystal violet to determine colony growth. “With DOX” indicates that cells were treated with 0.5 μg/mL doxycycline (DOX) for 72 hours. Cells not treated with DOX (“Without DOX”) are provided as a control.

FIG. 2E is a graph showing the log 2 fold change in expression of a MAPK target gene set determined by mRNA sequencing in cancer cells treated with shARAF, shBRAF, or shCRAF. Cells were treated with 0.5 μg/mL doxycycline (DOX) for 72 hours. Cells were treated with a MEK inhibitor (GDC-0973) for 6 hours, 250 nM (n=3) or an ERK inhibitor (GDC-0994). Data are normalized to a non-targeting control (shNT) and DMSO (no DOX).

FIG. 3A is a set of photomicrographs showing a Western blot analysis of the MAPK pathway components ARAF, BRAF, RAF1 (CRAF), pMEK, MEK, pERK, ERK, and a β actin control in A549 xenograft tumors treated with shCRAF or a non-targeting shRNA (shNT). Each lane represents a tumor from an individual animal (n=4 tumors per condition). DOX (+) indicates that cells were treated with 0.5 μg/mL doxycycline (DOX) for 72 hours.

FIG. 3B is a set of photomicrographs showing a Western blot analysis of the MAPK pathway components ARAF, BRAF, RAF1 (CRAF), pMEK, MEK, pERK, ERK, and a β actin control in CALU6 xenograft tumors treated with shCRAF or a non-targeting shRNA (shNT). Each lane represents a tumor from an individual animal (n=4 tumors per condition). DOX (+) indicates that cells were treated with 0.5 μg/mL doxycycline (DOX) for 72 hours.

FIG. 3C is a set of photomicrographs showing a Western blot analysis of the MAPK pathway components ARAF, BRAF, RAF1 (CRAF), pMEK, MEK, pERK, ERK, and a β actin control in SW620 xenograft tumors treated with shCRAF or a non-targeting shRNA (shNT). Each lane represents a tumor from an individual animal (n=4 tumors per condition). DOX (+) indicates that cells were treated with 0.5 μg/mL doxycycline (DOX) for 72 hours.

FIG. 3D is a set of photomicrographs showing representative immunostaining with anti-RAF1 (CRAF), anti-Cleaved Caspase 3, anti-p21 and anti-Ki67 antibodies in paraffin-embedded A549 xenograft tumors treated with shCRAF. Cells not treated with DOX are provided as a control.

FIG. 3E is a set of photomicrographs showing representative immunostaining with anti-RAF1 (CRAF), anti-Cleaved Caspase 3, anti-p21 and anti-Ki67 antibodies in paraffin-embedded CALU6 xenograft tumors treated with shCRAF. Cells not treated with DOX are provided as a control.

FIG. 3F is a set of photomicrographs showing representative immunostaining with anti-RAF1 (CRAF), anti-Cleaved Caspase 3, anti-p21 and anti-Ki67 antibodies in paraffin-embedded SW620 xenograft tumors treated with shCRAF. Cells not treated with DOX are provided as a control.

FIG. 3G is a box-and-whisker plot showing relative mRNA expression of RAF1 (CRAF), PUMA, p21, DUSP6, and SPRTY (as measured using qRT-PCR) in A549 xenograft tumors treated with shCRAF (n=4 tumors per condition). Cells not treated with DOX are provided as a control.

FIG. 3H is a box-and-whisker plot showing relative mRNA expression of RAF1 (CRAF), PUMA, p21, DUSP6, and SPRTY (as measured using qRT-PCR) in CALU6 xenograft tumors treated with shCRAF (n=4 tumors per condition). Cells not treated with DOX are provided as a control.

FIG. 3I is a box-and-whisker plot showing relative mRNA expression of RAF1 (CRAF), PUMA, p21, DUSP6, and SPRTY (as measured using qRT-PCR) in SW620 xenograft tumors treated with shCRAF (n=4 tumors per condition). Cells not treated with DOX are provided as a control.

FIG. 4A is a schematic diagram showing the domains of RAF1 (CRAF), the locations of the S529A, K375M, and D468N mutations, and the lengths of the N-terminal fragment (amino acids (aa) 1-303) and C-terminal (kinase domain) fragment (aa 303-648).

FIG. 4B is a set of photomicrographs showing the results of a soft agar colony formation growth assay for A549 cells (parental) or RAF1 (CRAF) knockout (KO) cancer cells expressing wild-type RAF1 (CRAF) or versions of RAF1 (CRAF) having S259A, D468N, D468A, or K375M mutations. The RAF1 (CRAF) mutants are expressed upon treatment with doxycycline. Colonies were stained with MTT dye and imaged using a GelCount imager. “With DOX” indicates that cells were treated with 0.25 μg/mL doxycycline (DOX) for 48 hours.

FIG. 4C is a set of photomicrographs showing the results of immunoprecipitation of FLAG-tagged wild-type RAF1 (CRAF) and FLAG-tagged versions of RAF1 (CRAF) having S259A, D468N, D468A, or K375M mutations in A549 RAF1 (CRAF) knockout cells. DOX (+) indicates that cells were treated with 0.25 μg/mL doxycycline for 48 hours. Eluates were co-immunoprecipitated and blotted with the indicated antibodies. GDC-0973 (+) indicates that cells were co-treated with the MEK inhibitor GDC-0973 (24 hours, 250 nM). The RAF1 (CRAF) immunoprecipitates were additionally analyzed for kinase activity (kinase assay) using inactive MEK as a substrate.

FIG. 4D is a set of photomicrographs showing the results of a soft agar colony formation growth assay for A549 cells (parental) or RAF1 (CRAF) knockout (KO) cancer cells expressing the RAF1 (CRAF) N-terminal domain (CRAF^{NTD}, aa 1-303), the RAF1 (CRAF) kinase domain (CRAF^{KD}, aa 303-648), or RAF1 (CRAF) kinase domains having kinase-dead mutations (CRAF^{K375M;KD} or CRAF^{D468N;KD}. Colonies were stained with MTT dye and imaged using a GelCount imager. “With DOX” indicates that cells were treated with 0.25 μg/mL doxycycline (DOX) for 48 hours.

FIG. 4E is a set of plots showing the average sum peptide spectrum matches (PSMs) and SAINT log odds scores for interacting partners of RAF1^{KD} (CRAF^{KD}) (left plot), RAF1^{KD,K375M} (CRAF^{KD,K375M}) (center plot), and RAF1^{KD,D468N} (CRAF^{KD,D468N}) (right plot) as determined using affinity purification mass spectrometry (AP-MS). RAF1 (CRAF) interaction partners are denoted based on average spectral counts (n=3) marked by a red dot to show significance (Bayesian false discovery rate (BDFR)<0.05).

FIG. 4F is a schematic diagram showing a representative workflow for applying the protein interaction, kinetics, and estimation of stoichiometries (PIKES) approach to RAF1 (CRAF) −/− A549 RASK (KRAS) mutant cells comprising wild-type RAF1 (CRAF), RAF1^{D468N} (CRAF^{D468N}), or RAF1^{K375M}(CRAF^{K375M}). Following lysis, immunoprecipitation (IP) using FLAG-RAF1 (FLAG-CRAF), and elution, a MAPK QCONCAT polypeptide is added to the sample. The sample is digested and mass spectrometry and parallel reaction monitoring (PRM) analyses are performed. Ratios of heavy (QCONCAT-derived) to light (sample-derived) polypeptides are calculated.

FIG. 4G is a box plot showing the absolute amount of FLAG-RAF1 (FLAG-CRAF) (fmol/IP) or a FLAG-empty control bound to ARAF or BRAF as measured by a PIKES targeted analysis comprising expression and immunoprecipitation of FLAG-tagged RAF1^{WT} (CRAF^{WT}) in RAF1 (CRAF) knockout A549 cells treated with 0.25 μg/mL DOX for 48 hours (n=3).

FIG. 4H is a box plot showing the heterodimerization efficiency of RAF1^{KD,K375M} (CRAF^{KD,K375M}) with ARAF and BRAF, represented as the average fold change in the amount of RAF1^{KD,K375M}(CRAF^{KD,K375M}) bound to ARAF or BRAF over CRAF^{WT} as measured by a PIKES targeted analysis in RAF1 (CRAF) knockout A549 cells

FIG. 4I is a box plot showing the heterodimerization efficiency of RAF1^{KD,D468N} (CRAF^{KD,D468N}) with ARAF and BRAF, represented as the average fold change in the amount of RAF1^{KD,D468N} (CRAF^{KD,D468N}) bound to ARAF or BRAF over RAF1^{WT} (CRAF^{WT}) as measured by a PIKES targeted analysis in RAF1 (CRAF) knockout A549 cells

FIG. 4J is a set of photomicrographs showing the results of a soft agar colony formation growth assay for A549 cells (parental) or ARAF knockout (KO), BRAF KO, or RAF1 (CRAF) KO cancer cells treated with the pan-RAF inhibitor AZ-628 at 100 nm, 1 μM, and 10 μM concentrations. Colonies were stained with MTT dye and imaged using a GelCount imager.

FIG. 4K is a schematic diagram of protein-protein interactions showing RAF1 (CRAF) preferential heterodimerization promoting RAF1 (CRAF) kinase-dependent or kinase-independent function.

FIG. 5A is a set of photomicrographs showing a Western blot analysis of the MAPK pathway components ARAF, BRAF, RAF1 (CRAF), pERK, ERK, and a β actin control in A549 parental cells or ARAF KO, BRAF KO, or RAF1 (CRAF) KO cells.

FIG. 5B is a set of photomicrographs showing the results of a soft agar colony formation growth assay for A549 cells (parental) or ARAF KO, BRAF KO, or RAF1 (CRAF) KO cells. Colonies were stained with MTT dye and imaged using a GelCount imager.

FIG. 5C is a set of photomicrographs showing a Western blot analysis of the MAPK pathway components ARAF, BRAF, RAF1 (CRAF), pMEK, MEK, pERK, ERK, and a β actin control in A549 parental or RAF1 (CRAF) knockout cells expressing CRAF^{S259A}, CRAF^{D468N}, CRAF^{D486A}, or CRAF^{K375M} constructs. DOX (+) indicates that cells were treated with 0.25 μg/mL doxycycline for 48 hours.

FIG. 5D is a box and whisker plot showing the number of colonies per well (mean±SEM, n=5) in a soft agar colony formation growth assay for A549 cells (parental) or RAF1 (CRAF) knockout cells expressing wild-type RAF1 (CRAF), RAF1^{S259A} (CRAF^{S259A}), RAF1^{D468N} (CRAF^{D468N}), RAF1^{D486}A (CRAF^{D486A}), or RAF1^{K375M} (CRAF^{K375M}) constructs. An empty vector control is provided. Colonies were stained with MTT dye and imaged using a GelCount imager.

FIG. 5E is a set of photomicrographs showing a Western blot analysis of the MAPK pathway components ARAF, BRAF, RAF1 (CRAF), pMEK, MEK, pERK, ERK, and a β actin control in A549 parental or RAF1 (CRAF) knockout cells expressing wild-type RAF1 (CRAF), RAF1^{D468A} (CRAF^{D468A}) or RAF1^{K375M} (CRAF^{K375M}) constructs. DOX (+) indicates that cells were treated with 0.25 μg/mL doxycycline for 48 hours. GDC-0973 (+) indicates that cells were treated with 250 nM of the MEK inhibitor GDC-0973 for 24 hours. An empty vector control is provided.

FIG. 5F is a set of photomicrographs showing the results of a long-term colony formation assay in wild-type A549 cells (parental) or RAF1 (CRAF) knockout cells expressing the indicated RAF1 (CRAF) mutants. Cells were treated with a DMSO control or 50 nM, 100 nM, or 250 nM of the MEK inhibitor GDC-0973 for 10 days. Cells were stained with crystal violet to determine colony growth.

FIG. 5G is a set of photomicrographs showing a Western blot analysis of the MAPK pathway components ARAF, BRAF, RAF1^{C-term} (CRAF^{C-term}), RAF1^{N-term} (CRAF^{N-term}), pMEK, MEK, pERK, ERK, and a β actin control in A549 parental or RAF1 (CRAF) knockout cells expressing RAF1^{KD} (CRAF^{KD}), RAF1^{D486N;KD} (CRAF^{D486N;KD}), or RAF1^{K375M;KD} (CRAF^{K375M;KD}) constructs. DOX (+) indicates that cells were treated with 0.25 μg/mL doxycycline for 48 hours. An empty vector control is provided. RAF1 (CRAF) N-terminal and C-terminal deletion mutants were detected with specific RAF1 (CRAF) antibodies.

FIG. 5H is a schematic diagram showing a representative workflow for applying an affinity purification-mass spectrometry (AP-MS) approach to identify binding partners of RAF1^{WT;KD} (CRAF^{WT;KD}) RAF1^{D486N;KD} (CRAF^{D486N;KD}), or RAF1^{K375M;KD} (CRAF^{K375M;KD}). Following lysis, immunoprecipitation (IP) using FLAG-CRAF, and elution, the sample is digested and mass spectrometry and data-dependent acquisition (DDA) analyses are performed.

FIG. 6A is a sequence alignment of the human RAS isoforms RASH (SEQ ID NO: 93), RASK) SEQ ID NO: 94), and RASN (SEQ ID NO: 95). The distinguishing and shared peptides used to identify protein abundance of each isoform are indicated by bolded residues.

FIG. 6B is a sequence alignment of the human RAF isoforms BRAF (SEQ ID NO: 96), ARAF (SEQ ID NO: 97), and RAF1 (SEQ ID NO: 98). The distinguishing and shared peptides used to identify protein abundance of each isoform are indicated by bolded residues.

FIG. 6C is a box-and-whisker plot showing the expression level (log₂ nRPKM) of the RAF isoforms ARAF, BRAF, and RAF1 (CRAF) in all RASK (KRAS) mutant cells represented in the Achilles dataset.

FIG. 6D is a bar graph showing the expression level (RPKM) of the RAS isoforms RASK, RASN, and RASH and the RAF isoforms ARAF, BRAF, and RAF1 (CRAF) in the indicated RASK (KRAS) mutant cancer cells.

FIG. 6E is a bar graph showing the protein abundance (fmol per μg extract) of a shared RAF polypeptide (panRAS^{WT}), mutant KRAS^{G12/G13}, and the RAS isoforms RASH (HRAS), RASN (NRAS), and RASK (KRAS) in the indicated RASK (KRAS) mutant cells (mean±SEM, n=3) as measured using PIKES-based quantification of relative protein abundance.

FIG. 6F is a bar graph showing the protein abundance (fmol per μg extract) of a shared RAF polypeptide (panRAF) and the RAF isoforms ARAF, BRAF, and RAF1 (CRAF) in the indicated RASK (KRAS) mutant cells (mean±SEM, n=3) as measured using PIKES-based quantification of relative protein abundance upon expression and immunoprecipitation of FLAG-tagged CRAF^{WT} in CRAF knockout A549 cells (0.25 μg/ml doxycycline for 48 hours). CRAF dimerization partners are denoted by the absolute amount of CRAF bound (fmol/IP).

FIG. 6G is a set of photomicrographs showing the results of a soft agar colony formation growth assay for the indicated cancer cells treated with non-targeting shRNAs (shNT) or shRNA targeting ARAF (shARAF), BRAF (shBRAF), or RAF1 (CRAF) (shCRAF) and treated with the pan-RAF inhibitor AZ-628 at 100 nm, 1 μM, and 10 μM concentrations or a DMSO control. “With DOX” indicates that cells were treated with 0.5 μg/mL doxycycline for 48 hours.

FIG. 7A is a bar graph showing the CERES correlation in the Achilles data set of the indicated genes with BRAF and RAF1 (CRAF).

FIG. 7B is a set of photomicrographs showing the results of a soft agar colony formation growth assay for the indicated cancer cells treated with non-targeting shRNAs (shNT) or a shRNA targeting SHOC2 (shSHOC2). “With DOX” indicates that cells were treated with 0.5 μg/mL doxycycline for 72 hours. The cells were stained with MTT reagent and imaged using the GelCount imager.

FIG. 7C is a set of photomicrographs showing a Western blot analysis of levels of SHOC2, pERK, and ERK in the indicated cancer cell lines following treatment with non-targeting shRNAs (shNT) or shSHOC2. DOX (+) indicates that cells were treated with (0.5 μg/mL doxycycline, for 72 hours).

FIG. 7D is bar graph showing relative levels of SHOC2 MRNA (as measured using qRT-PCR) in the indicated cancer cell lines treated with a non-targeting shRNA (shNT) or shSHOC2. DOX (+) indicates that cells were treated with 0.5 μg/mL doxycycline for 72 hours.

FIG. 7E is a set of photomicrographs showing the results of a soft agar colony formation growth assay in A549 ARAF knockout (ARAF KO) and BRAF knockout (BRAF KO) cancer cells treated with non-targeting shRNAs (shNT) or shSHOC2. “With DOX” indicates that cells were treated with 0.5 μg/mL doxycycline for 72 hours. The cells were stained with MTT reagent and imaged using the GelCount imager.

FIG. 7F is a set of photomicrographs showing a Western blot analysis of levels of SHOC2, ARAF, BRAF, RAF1 (CRAF), pMEK, MEK, pERK, and ERK and a β actin control in A549 ARAF knockout (ARAF KO) or BRAF knockout (BRAF KO) cancer cells treated with non-targeting shRNAs (shNT) or shSHOC2. DOX (+) indicates that cells were treated with 0.5 μg/mL doxycycline for 72 hours.

FIG. 7G is a set of photomicrographs showing a Western blot analysis of levels of SHOC2, ARAF, BRAF, RAF1 (CRAF), pERK, and ERK and a β actin control in A549 BRAF knockout (BRAF KO) cancer cells treated with non-targeting shRNAs (shNT) or shSHOC2 and stimulated with EGF (50 ng/μl) for the indicated durations. DOX (+) indicates that cells were treated with 0.5 μg/mL doxycycline for 72 hours.

FIG. 7H is a set of photomicrographs showing the results of immunoprecipitation (IP) of endogenous RAF1 (CRAF) in the A549 BRAF knockout cancer cells upon treatment with shNT or shSHOC2. Cells were treated with 0.5 μg/mL doxycycline for 72 hours. RAF1 (CRAF) immunoprecipitates were co-immunoprecipitated with the indicated antibodies. EGF (+) indicates that cells were stimulated with EGF (50 ng/μl for 10 minutes).

FIG. 7I is a set of photomicrographs showing the results of immunoprecipitation (IP) of endogenous ARAF in the A549 BRAF knockout cancer cells upon treatment with shNT or shSHOC2. Cells were treated with 0.5 μg/mL doxycycline for 72 hours. RAF1 (CRAF) immunoprecipitates were co-immunoprecipitated with the indicated antibodies. EGF (+) indicates that cells were stimulated with EGF (50 ng/μl for 10 minutes).

FIG. 8A is a set of photomicrographs showing the results of a soft agar colony formation growth assay in the indicated cancer cell lines treated with shRNAs targeting RAF1 (CRAF) (shCRAF) and luciferase (shLuciferase) or shRNAs targeting RAF1 (CRAF) (shCRAF) and ARAF (shARAF). “With DOX” indicates that cells were treated with 0.5 μg/mL doxycycline. The cells were stained with MTT reagent and imaged using the GelCount imager.

FIG. 8B is a set of photomicrographs showing the results of a soft agar colony formation growth assay in A549 parental, RAF1 (CRAF) knockout (CRAF KO), BRAF and RAF1 (CRAF) knockout (BRAF KO;CRAF KO), ARAF and RAF1 (CRAF) knockout (ARAF KO;CRAF KO), and ARAF and BRAF knockout (ARAF KO;BRAF KO) cell lines. The cells were stained with MTT reagent and imaged using the GelCount imager.

FIG. 8C is a set of photomicrographs showing the results of a soft agar colony formation growth assay in A549 parental, ARAF knockout (ARAF KO), RAF1 (CRAF) knockout (CRAF KO), and ARAF and RAF1 (CRAF) knockout (ARAF KO;CRAF KO) cell lines expressing either a luciferase control or the indicated wild-type (WT) or mutant forms of ARAF and RAF1 (CRAF). Cells were treated with 0.5 μg/mL doxycycline for 10 days.

FIG. 8D is a set of photomicrographs showing the results of immunoprecipitation (IP) of ARAF and BRAF, followed by a kinase assay, in A549 cells treated with non-targeting shRNA (shNT) or shCRAF. DOX (+) indicates that cells were treated with 0.5 μg/mL doxycycline for 72 hours. The eluates were co-immunoprecipitated and blotted with the indicated antibodies. The ARAF and BRAF immunoprecipitates were analyzed for kinase activity utilizing inactive MEK as a substrate.

FIG. 8E is a set of photomicrographs showing the results of immunoprecipitation (IP) of ARAF and, followed by a kinase assay, in A549 cells (parental), and RAF1 (CRAF) knockout (CRAF KO), and BRAF and RAF1 (CRAF) knockout (BRAF KO/CRAF KO) cells stimulated with EGF (50 ng/μl, 10 minutes). Right top panel: Immunoprecipitation of ARAF was performed and lysates were immunoblotted with the indicated antibodies.

FIG. 9A is a set of photomicrographs showing a Western blot analysis of levels of RAF1 (CRAF), BRAF, ARAF, pMEK, MEK, pERK, ERK, pRSK, RSK, and a β actin control in the indicated cancer cell lines treated with non-targeting shRNAs (shNT), shCRAF, or shARAF. DOX (+) indicates that cells were treated with 0.5 μg/mL doxycycline for 72 hours.

FIG. 9B is a plot showing the number of colonies counted per well in a soft agar colony formation growth assay in the indicated cancer cell lines treated with non-targeting shRNAs (shNT), shCRAF, and shRNAs targeting RAF1 (CRAF) and ARAF (shCRAF;shARAF). The colonies were stained with MTT dye and imaged using the Gel Count imager (mean±SEM, n=6).

FIG. 9C is a set of photomicrographs showing a Western blot analysis of levels of RAF1 (CRAF), ARAF, BRAF, pMEK, MEK, pERK, ERK, pRSK, RSK, and a β actin control in A549 parental, ARAF knockout (ARAF KO), BRAF knockout (BRAF KO), RAF1 (CRAF) knockout (CRAF KO) and ARAF and RAF1 (CRAF) double knockout (ARAF;CRAF KO) cells expressing the indicated mutants. DOX (+) indicates that cells were treated with 0.5 μg/mL doxycycline for 48 hours.

FIG. 9D is a graph showing the corrected FRET ratio (A.U.) measured in an in vitro TR-FRET MP2K1 (MEK1) phosphorylation assay upon ATP titration of the indicate RAF:14-3-3 dimers or a BRAF^{V600E} monomer (mean±SEM, n=2).

FIG. 10A is a schematic diagram showing RAF1 (CRAF) conditional knock-out (RAF1^{fl/fl} (CRAF^{fl/fl})) and RAF1 (CRAF) conditional knock-in (RAF1^{D468A} (CRAF^{D468A}) and RAF1^{R401H} (CRAF^{R401H})) mice and the observed phenotype (embryonic lethal or not embryonic lethal (alive)).

FIG. 10B is a set of plots showing the results of a digital drop genotyping PCR assay using the RAF1 (CRAF) wild-type, RAF1^{R401H} (CRAF^{R401H}), and RAF1^{D468A} (CRAF^{D468A}) primers to validate the expression of RAF1 (CRAF) and the indicated mutants (n=5 mice).

FIG. 10C is a representative table indicating the Mendelian ratios observed for the expression of RAF1 (CRAF) mutants (RAF1^{D468A} (CRAF^{D468A}) and RAF1^{R401H} (CRAF^{R401H})) in mice at the indicated timepoints.

FIG. 10D is a graph showing overall survival (percent survival) at 0-60 weeks in RAF1 (CRAF) wild-type (wt/wt; n=15), RAF1 (CRAF) D468A/+(wt/ki; n=15), and RAF1 (CRAF) D468A/D468A (ki/ki; n=15) mice.

FIG. 10E is a set of photomicrographs showing a Western blot analysis of levels of RAF1 (CRAF), pERK, ERK, a β actin control in the indicated tissues upon expression of WT RAF1 (CRAF), RAF1 (CRAF) KO, or RAF1 (CRAF) mutants (RAF1^{D468A} (CRAF^{D468A}) and RAF1^{R401H} (CRAF^{R401H})). Tissues were collected from (n=1) mouse for the Western blot analysis. A total of (n=10 mice) were enrolled in the study.

FIG. 11A is a set of photomicrographs showing a Western blot analysis of levels of ARAF, BRAF, RAF1 (CRAF), pMEK, MEK, pERK, ERK, and a β actin control in RAF1 (CRAF) KO, BRAF KO;RAF1 (CRAF) KO, ARAF KO; RAF1 (CRAF) KO, and BRAF KO CRISPR knockout clones following EGF stimulation (50 ng/μl) at 0, 5, 10, 15, 30, and 60 minutes.

FIG. 11B is a graph showing the ratio of pMEK to total MEK (A.U.) as measured using a meso scale discovery (MSD) plate assay for MEK phosphorylation in the A549 parental and indicated CRISPR knockout clones upon EGF stimulation (50 ng/μl) at the indicated time points (mean±SEM, n=3).

FIG. 11C is a graph showing the ratio of pERK to total ERK (A.U.) as measured using a meso scale discovery (MSD) plate assay for ERK phosphorylation in the A549 parental and indicated CRISPR knockout clones upon EGF stimulation (50 ng/μl) at the indicated time points (mean±SEM, n=3).

FIG. 11D is a set of photomicrographs showing a Western blot analysis of levels of p21, cleaved caspase 3, and a β actin control in CALU6 cells upon treatment with non-targeting shRNAs (shNT) or shCRAF. DOX (+) indicates that cells were treated with 0.5 μg/mL doxycycline for 72 hours. GDC-0973 (+) indicates that cells were co-treated with a MEK inhibitor (GDC-0973, 250 nM, 24 hours). The lysates were probed for the indicated antibodies.

FIG. 11E is a set of photomicrographs showing a Western blot analysis of levels of RAF1 (CRAF), ARAF, BRAF, pERK, ERK, pRSK, RSK, p21, and a β actin control in A549 cells upon treatment with non-targeting shRNAs (shNT), shCRAF, or shCRAF;shARAF. DOX (+) indicates that cells were treated with 0.5 μg/mL doxycycline for 72 hours. The lysates were probed for the indicated antibodies.

FIG. 11F is a set of photomicrographs showing the results of a soft-agar colony formation assay in A549 parental and RAF1 (CRAF) knockout (CRAF KO) cancer cells upon treatment with DMSO or a MEK inhibitor (GDC-0973) at the indicated dose titrations. The colonies were labeled with MTT reagent and imaged using a GelCount imager.

FIG. 11G is a set of photomicrographs showing the results of a soft-agar colony formation assay in A549 parental and RAF1 (CRAF) knockout (CRAF KO) cancer cells upon treatment with DMSO or an ERK inhibitor (GDC-0994) at the indicated dose titrations. The colonies were labeled with MTT reagent and imaged using a GelCount imager.

FIG. 11H is a set of schematic diagrams of protein-protein interactions showing dimerization-dependent functions in promotion of RASK (KRAS) tumorigenesis.

FIG. 12A is a set of photomicrographs showing the results of a soft-agar colony formation assay in HCT116 parental, p21 knockout (p21−/−), PUMA knockout (PUMA^{-/-}), PUMA and p21 double knockout (PUMA^{-/-};p21−/−), and BAX and BAK double knockout (BAX^{-/-};Bak^{-/-}) cells upon treatment with non-targeting shRNAs (shNT) or shCRAF. “With DOX” indicates that cells were treated with 0.5 μg/mL doxycycline for 10 days. Colonies were labelled with MTT dye and imaged using a GelCount imager.

FIG. 12B is a set of photomicrographs showing a Western blot analysis of levels of RAF1 (CRAF), pCRAF, pERK, ERK, PUMA, p21, and a β actin control in HCT116 parental, p21 knockout (p21^{-/-}), PUMA knockout (PUMA^{-/-}), PUMA and p21 double knockout (PUMA^{-/-};p21−/−) cells following treatment with non-targeting shRNAs (shNT) or shCRAF. DOX (+) indicates that cells were treated with 0.5 μg/mL doxycycline for 72 hours.

FIG. 12C is a set of photomicrographs showing a Western blot analysis of levels of RAF1 (CRAF), BAX, pERK, ERK, and a β actin control in HCT116 parental and BAX and BAK double knockout (BAX^{-/-};Bak^{-/-}) cells following treatment with non-targeting shRNAs (shNT) or shCRAF. DOX (+) indicates that cells were treated with 0.5 μg/mL doxycycline for 72 hours.

FIG. 12D is a bar graph showing the relative level of RAF1 (CRAF) mRNA (as measured using qRT-PCR) in HCT116 RASK (KRAS) mutant parental or BAX and BAK double knockout (BAX^{-/-};Bak^{-/-}) upon treatment with non-targeting shRNAs (shNT) or shCRAF. DOX (+) indicates that cells were treated with 0.5 μg/mL doxycycline for 72 hours.

FIG. 12E is a bar graph showing the relative level of BAX mRNA (as measured using qRT-PCR) in HCT116 RASK (KRAS) mutant parental or BAX and BAK double knockout (BAX^{-/-};Bak^{-/-}) upon treatment with non-targeting shRNAs (shNT) or shCRAF. DOX (+) indicates that cells were treated with 0.5 μg/mL doxycycline for 72 hours.

FIG. 12F is a bar graph showing the relative level of BAK mRNA (as measured using qRT-PCR) in HCT116 RASK (KRAS) mutant parental or BAX and BAK double knockout (BAX^{-/-};Bak^{-/-}) upon treatment with non-targeting shRNAs (shNT) or shCRAF. DOX (+) indicates that cells were treated with 0.5 μg/mL doxycycline for 72 hours.

FIG. 12G is a set of photomicrographs showing the results of a soft-agar colony formation assay in A549 cancer cells upon treatment with shCRAF and co-treatment with the indicated inhibitors. “With DOX” indicates that cells were treated with 0.5 μg/mL doxycycline for 10 days. Cells were co-treated with DMSO or inhibitors for cellular apoptosis (ZVAD-FMK, 20 μM), necroptosis (NEC1, 30 μM) or a combination of both (ZVAD-FMK, 10 μM; NEC1, 10 μM) and an autophagy inhibitor (Bafilomycin, 50 nM).

FIG. 12H is a set of photomicrographs showing a Western blot analysis of levels of the indicated proteins in CALU6 cells treated with non-targeting shRNAs (shNT) or shCRAF. DOX (+) indicates that cells were treated with 0.5 μg/mL doxycycline. GDC-0973 (+) indicates that cells were treated with a MEK inhibitor (GDC-0973, 250 nM, for 24 hours).

FIG. 12I is a heat map showing the results of a gene set enrichment analysis (GSEA) of apoptosis target genes upon acute depletion using shARAF, shBRAF, and shCRAF. Cells were treated with 0.25 μg/mL doxycycline for 72 hours. Differential analysis was performed by normalizing the data to non-targeting shRNA (shNT).

FIG. 13 is a box plot showing the absolute amount of CRAF^{WT} in cells treated with the MEK inhibitor GDC-0973 (250 nM, 24 hours) as measured by a PIKES targeted analysis comprising expression and immunoprecipitation of FLAG-tagged RAF1^{WT} (CRAF^{WT}).

### DETAILED DESCRIPTION OF THE INVENTION

### 1. Definitions

As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) aspects that are directed to that value or parameter per se. For example, description referring to “about X” includes description of “X.” In some embodiments, “about” may refer to ±15%, ±10%, ±5%, or ±1% as understood by a person of skill in the art.

It is understood that aspects of the invention described herein include “comprising,” “consisting,” and “consisting essentially of” aspects.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, plasma, serum, blood-derived cells, urine, cerebro-spinal fluid, saliva, buccal swab, sputum, tears, perspiration, mucus, tumor lysates, and tissue culture medium, tissue extracts such as homogenized tissue, tumor tissue, cellular extracts, and combinations thereof. The sample may be an archival sample, a fresh sample, or a frozen sample. In some aspects, the sample is a formalin-fixed and paraffin-embedded (FFPE) tumor tissue sample.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

A “subject” or an “individual” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the subject or individual is a human.

### II. Recombinant Proteins

Provided herein are recombinant proteins comprising a set of non-identical, contiguous polypeptides consisting of sequences present in members of a signaling pathway of interest (e.g., members of the of the mitogen activated protein kinase (MAPK) pathway or the phosphoinositide 3-kinase (PI3K) pathway), wherein each polypeptide of the set may be separated upon exposure of the recombinant protein to a cleavage agent. Separation produces an equimolar set of polypeptides that may be used as an internal standard for quantification of protein levels in a sample.

One method for using such recombinant proteins for proteomic profiling is the PIKES (Protein Interaction (label-free mass spectrometry (MS)), Kinetics (stable isotope labeling by amino acids in cell culture (SILAC) MS), Estimation of Stoichiometries (parallel reaction monitoring (PRM) MS)) approach, described, e.g., in Reichermeier et al., Mol Cell, 77: 1092-1106 el 099, 2020. Each recombinant protein is converted into equimolar ratios of polypeptides consisting of a sequence present in a target gene (internal standard polypeptides), which can be used to distinguish between and estimate the stoichiometries of closely related target genes. This method allows absolute quantification of protein levels, thus allowing intra-sample comparison.

In some aspects, the recombinant protein in may be translated in vitro. In some aspects, the recombinant protein is purified, e.g., from a population of cultured cells. In some aspects, the recombinant protein is at least 80% pure, 85% pure, 90% pure, 95% pure, 97% pure, 98% pure, 99% pure, or more than 99% pure.

### A. Recombinant Protein Design

i. Selection of Polypeptides

In some aspects, the recombinant protein includes at least 5, at least 10, at least 15, at least 20, at least 25, at least 30, at least 35, at least 40, at least 45, or about 50 polypeptides. A polypeptide may consist of a sequence present in only one sequence in the set of proteins to be quantified (target proteins), e.g., may be a unique sequence, or may consist of a sequence present in two or more of the set of target proteins. In some aspects, the recombinant protein includes more than one (e.g., two, three, four, or five) distinct polypeptides corresponding to a single target protein. In some aspects, the recombinant protein includes one or more polypeptides for use as a control, e.g., one or more polypeptides consisting of a sequence present in a control protein, e.g., G3P or ACTA. In some aspects, each of the polypeptides is between 6 and 25 amino acid residues in length, e.g., is 6, 7, 8, 9, 10, 11, 12, 13, 14, 15, 16, 17, 18, 19, 20, 21, 22, 23, 24, or 25 amino acid residues in length. Multiple arrangements of polypeptides in the recombinant protein are contemplated. General principles for selecting polypeptides from target proteins are described, e.g., in Pratt et al., Nature Protocols, 1(2): 1029-1043, 2006.

ii. Cleavage Sites

In some aspects, a recombinant protein comprises a cleavage site between each polypeptide of the set that allows separation of each polypeptide upon exposure of the recombinant protein to a cleavage agent. In some aspects, the cleavage site is a trypsin cleavage site (e.g., the polypeptides are tryptic polypeptides) and the cleavage agent is trypsin. Methods for identifying tryptic polypeptides are known in the art. In other aspects, the cleavage agent is another endoprotease, e.g., endopeptidase ArgC, endopeptidase LysC, chymotrypsin, endopeptidase Asp-N, staphylococcal peptidase I, or trypsin. Further examples of cleavage agents include chemical cleavage agents, e.g., cyanogen bromide. Cleavage reagents and methods of selecting a cleavage strategy for a recombinant protein are described, e.g., in Pratt et al., Nature Protocols, 1(2): 1029-1043, 2006.

iii. 5′ and 3′ Regions

In some aspects, a recombinant protein comprises an N-terminal sequence comprising methionine (e.g., a nucleic acid encoding the recombinant protein comprises a transcriptional start site) and a cleavage site (e.g., a trypsin cleavage site) between the N-terminal sequence and the set of polypeptides that allows separation of the N-terminal sequence from the set of polypeptides upon exposure of the recombinant protein to a cleavage agent (e.g., trypsin). In some aspects, the N-terminal sequence has the amino acid sequence of SEQ ID NO: 88.

In some aspects, a recombinant protein comprises a C-terminal sequence comprising one or more tags that may be used for purification of the recombinant protein, e.g., a polyhistidine tag (e.g., a tag having the amino acid sequence of SEQ ID NO: 89). The tag may be, e.g., a FLAG tag (e.g., a FLAG tag comprising the amino acid sequence of SEQ ID NO: 90), a HA tag (e.g., a HA tag comprising the amino acid sequence of SEQ ID NO: 91), or a V5 tag (e.g., a V5 tag comprising the amino acid sequence of SEQ ID NO: 92). In some aspects, the tag is a tandem tag, e.g., a 2×, 3×, 4×, 5×, 6×, 7×, or 8×FLAG tag, His tag, or HA tag. In some aspects, the tandem tag is a 3×tag. Tandem tags may be heterogeneous, e.g., may comprise two or more of a FLAG tag, a HA tag, or V5 tag. In some aspects, the tandem tag is a His-FLAG tag. The C-terminal sequence may further comprise a cleavage site (e.g., a trypsin cleavage site) between the C-terminal sequence and the set of polypeptides that allows separation of the C-terminal sequence from the set of polypeptides upon exposure of the recombinant protein to a cleavage agent (e.g., trypsin).

iv. Labeling of Recombinant Proteins

In some aspects, each polypeptide of the set of non-identical, contiguous polypeptides comprised by a recombinant protein comprises a label. The label may be any moiety that can be used to distinguish a polypeptide derived from the recombinant protein from a corresponding, un-labeled polypeptide derived from the sample. Labeling strategies for recombinant proteins are described, e.g., in Pratt et al., Nature Protocols, 1(2): 1029-1043, 2006.

In some aspects, the label is an isotopic label. In some aspects, the isotopic label is heavy arginine, e.g., ¹³C₁-arginine (R1); ¹³C₂-arginine (R2); ¹⁵N₄-arginine (R4); ¹³C₆-arginine (R6); ²H₇- arginine (R7); ¹³C₆, ¹⁵N₄-arginine (R10); ²H₇, ¹⁵N₄-arginine (R11), or ¹³C₆, ²H₇, ¹⁵N₄-arginine (R17). Representative heavy arginine species are provided in Table 1. In some aspects, the isotopic label is heavy lysine, e.g., ¹³C₁-lysine (K1); ¹⁵N₂-lysine (K2); ²H₄-lysine (K4); ¹³C₆-lysine (K6); ¹³C₆, ¹⁵N₂-lysine (K8); ²H₈-lysine (K8); ²H₉-lysine (K9); ²H₉, ¹⁵N₂-lysine (K11); or ¹³C₆; ²H₉, ¹⁵N₂-lysine (K17). Representative heavy lysine species are provided in Table 2. Further examples of lysine isotopic labels include the Thermo Fisher NEUCODE™ lysines K521, K440, 390, 642, 192, and 202 (13C, 2H, 15N). Other isotopic labels are also contemplated, including those on amino acids other than arginine and lysine.

TABLE 1
Arginine isotopes

| R# | ¹³C | ²H (Deuterated) | ¹⁵N |
|---|---|---|---|
| R1 | 1 | | |
| R2 | 2 | | |
| R4 | | | 4 |
| R6 | 6 | | |
| R7 | | 7 | |
| R10 | 6 | | 4 |
| R11 | | 7 | 4 |
| R17 | 6 | 7 | 4 |

TABLE 2
Lysine isotopes

| K# | ¹³C | ²H (Deuterated) | ¹⁵N |
|---|---|---|---|
| K1 | 1 | | |
| K2 | | | 2 |
| K4 | | 4 | |
| K6 | 6 | | |
| K8 | 6 | | 2 |
| K8 | | 8 | |
| K9 | | 9 | |
| K11 | | 9 | 2 |
| K17 | 6 | 9 | 2 |

In some aspects, the label is a chemical label. In some aspects, the chemical label is a tandem mass tag (TMT), an iTRAQ, a label produced by reductive methylation/dimethylation, or a label produced by acetylation (e.g., acetic anhydride). In some aspects, the label (e.g., chemical label) is an isobaric label.

In some aspects, the recombinant protein is at least 98% labeled. In some aspects, the recombinant protein is at least 99% labeled. In some aspects, the recombinant protein is 100% labeled.

### B. Recombinant Proteins Useful in Assessment of MAPK Pathway Components

Provided herein are recombinant proteins comprising a set of non-identical, contiguous polypeptides consisting of sequences present in one or more members of the mitogen activated protein kinase (MAPK) pathway, or mutant or variant forms thereof, e.g., sequences present in one or more of ARAF, BRAF, BRAF^{V600E}, RASH (HRAS), RASH^{Q61K}(HRAS^{Q61K}), RASH^{Q61R}(HRAS^{Q61R}), RASH^{G12V}(HRAS^{G12V}), RASH^{G13D} (HRAS^{G13D}), RASH^{G12C} (HRAS^{G12C}), RASH^{G12D} (HRAS^{G12D}), RASH^{G12S} (HRAS^{G12S}), RASK (KRAS), RASK^{Q61K} (KRAS^{Q61K}), RASK^{Q61R} (KRAS^{Q61R}), RASK^{G12V} (KRAS^{G12V}), RASK^{G13D} (KRAS^{G13D}), RASK^{G12C} (KRAS^{G12C}), RASK^{G12D} (KRAS^{G12D}), RASK^{G12S} (KRASGI²S), MP2K1 (MEK1), MP2K2 (MEK2), MK03 (ERK1/MAPK3), MK01 (ERK2/MAPK1), RASN (NRAS), RASN^{Q61K}(NRAS^{Q61K}), RASN^{Q61R} (NRAS^{Q61R}), RASN^{G12V} (NRAS^{G12V}), RASN^{G13D} (NRAS^{G13D}), RASN^{G12C} (NRAS^{G12C}), RASN^{G12D} (NRAS^{G12D}), RASN^{G12S} (NRAS^{G12S}), and RAF1 (CRAF).

In some aspects, provided herein is a recombinant protein comprising a set of non-identical, contiguous polypeptides, the set comprising one or more of a polypeptide consisting of a sequence present in RAF1; a polypeptide consisting of a sequence present in BRAF; a polypeptide consisting of a sequence present in BRAF^{V600E}; a polypeptide consisting of a sequence present in ARAF; a polypeptide consisting of a sequence present in MP2K1; a polypeptide consisting of a sequence present in MP2K2; a polypeptide consisting of a sequence present in MK03; a polypeptide consisting of a sequence present in MK01; a polypeptide consisting of a sequence present in RASK; a polypeptide consisting of a sequence present in RASN; a polypeptide consisting of a sequence present in RASH; a polypeptide consisting of a sequence present in each of RAF1, BRAF, and ARAF; a polypeptide consisting of a sequence present in both of RASH and RASN; a polypeptide consisting of a sequence present in both of RASN and RASK; a polypeptide consisting of a sequence present in each of RASH, RASN, and RASK; a polypeptide consisting of a sequence present in each of RASH^{Q61K}, RASN^{Q61K}, and RASK^{Q61K}; a polypeptide consisting of a sequence present in each of RASH^{Q61R}, RASN^{Q61R}, and RASK^{Q61R}; a polypeptide consisting of a sequence present in each of RASH^{G12V}, RASN^{G12V}, and RASK^{G12V}; a polypeptide consisting of a sequence present in each of RASH^{G13D}, RASN^{G13D}, and RASK^{G13D}; a polypeptide consisting of a sequence present in each of RASH^{G12C}, RASN^{G12C}, and RASK^{G12C}; a polypeptide consisting of a sequence present in each of RASH^{G12D}, RASN^{G12D}, and RASK^{G12D}; and a polypeptide consisting of a sequence present in each of RASH^{G12S}, RASN^{G12S}, and RASK^{G12S}, e.g., comprising at least 1, 2, 3, 4, 5, 6, 7, 8, 9, 10, 11, 12, 13, 14, 15, 16, 17, 18, 19, 20, 21, or all 22 polypeptides listed above, wherein the recombinant protein comprises a cleavage site (e.g., a trypsin cleavage site) between each polypeptide of the set that allows separation of each polypeptide upon exposure of the recombinant protein to a cleavage agent (e.g., trypsin).

In some aspects, provided herein is a recombinant protein comprising a set of non-identical, contiguous polypeptides, the set comprising a polypeptide consisting of a sequence present in RAF1 (CRAF); a polypeptide consisting of a sequence present in BRAF; a polypeptide consisting of a sequence present in BRAF^{V600E}; a polypeptide consisting of a sequence present in ARAF; a polypeptide consisting of a sequence present in MP2K1; a polypeptide consisting of a sequence present in MP2K2; a polypeptide consisting of a sequence present in MK03; a polypeptide consisting of a sequence present in MK01; a polypeptide consisting of a sequence present in RASK; a polypeptide consisting of a sequence present in RASN; a polypeptide consisting of a sequence present in RASH; a polypeptide consisting of a sequence present in each of RAF1, BRAF, and ARAF; a polypeptide consisting of a sequence present in both of RASH and RASN; a polypeptide consisting of a sequence present in both of RASN and RASK; a polypeptide consisting of a sequence present in each of RASH, RASN, and RASK; a polypeptide consisting of a sequence present in each of RASH^{Q61K}, RASN^{Q61K}, and RASK^{Q61K}; a polypeptide consisting of a sequence present in each of RASH^{Q61R}, RASN^{Q61R}, and RASK^{Q61R}; a polypeptide consisting of a sequence present in each of RASH^{G12V}, RASN^{G12V}, and RASK^{G12V}; a polypeptide consisting of a sequence present in each of RASH^{G13D}, RASN^{G13D}, and RASK^{G13D}; a polypeptide consisting of a sequence present in each of RASH^{G12C}, RASN^{G12C}, and RASK^{G12C}; a polypeptide consisting of a sequence present in each of RASH^{G12D}, RASN^{G12D}, and RASK^{G12D}; and a polypeptide consisting of a sequence present in each of RASH^{G12S}, RASN^{G12S}, and RASK^{G12S}, wherein the recombinant protein comprises a cleavage site (e.g., a trypsin cleavage site) between each polypeptide of the set that allows separation of each polypeptide upon exposure of the recombinant protein to a cleavage agent (e.g., trypsin).

In some aspects, each of the polypeptides is between 6 and 25 amino acid residues in length, e.g., is 6, 7, 8, 9, 10, 11, 12, 13, 14, 15, 16, 17, 18, 19, 20, 21, 22, 23, 24, or 25 amino acid residues in length.

In some aspects, the polypeptide consisting of a sequence present in BRAF^{V600E} has the amino acid sequence of SEQ ID NO: 10; the polypeptide consisting of a sequence present in each of RASH^{Q61K}, RASN^{Q61K}, and RASK^{Q61K} has the amino acid sequence of SEQ ID NO: 37; the polypeptide consisting of a sequence present in each of RASH^{Q61R}, RASN^{Q61R}, and RASK^{Q61R} has the amino acid sequence of SEQ ID NO: 38; the polypeptide consisting of a sequence present in each of RASH^{G12V}, RASN^{G12V}, and RASK^{G12V} has the amino acid sequence of SEQ ID NO: 39; the polypeptide consisting of a sequence present in each of RASH^{G13D}, RASN^{G13D}, and RASK^{G13D} has the amino acid sequence of SEQ ID NO: 40; the polypeptide consisting of a sequence present in each of RASH^{G12C}, RASN^{G12C}, and RASK^{G12C} has the amino acid sequence of SEQ ID NO: 41; the polypeptide consisting of a sequence present in each of RASH^{G12D}, RASN^{G12D}, and RASK^{G12D} has the amino acid sequence of SEQ ID NO: 42; and/or the polypeptide consisting of a sequence present in each of RASH^{G12S}, RASN^{G12S}, and RASK^{G12S} has the amino acid sequence of SEQ ID NO: 43.

In some aspects, the set of non-identical, contiguous polypeptides comprised by the recombinant protein includes at least two, at least three, or at least four non-identical polypeptides consisting of a sequence present in a target molecule. In some aspects, the set comprises at least two non-identical polypeptides consisting of a sequence present in RAF1, BRAF, ARAF, MP2K1, MP2K2, MK03, MK01, RASK, RASN, or RASH (e.g., at least two polypeptides consisting of a sequence present in RAF1, at least two polypeptides consisting of a sequence present in BRAF, at least two polypeptides consisting of a sequence present in ARAF, at least two polypeptides consisting of a sequence present in MP2K1, at least two polypeptides consisting of a sequence present in MP2K2, at least two polypeptides consisting of a sequence present in MK03, at least two polypeptides consisting of a sequence present in MK01, at least two polypeptides consisting of a sequence present in RASK, at least two polypeptides consisting of a sequence present in RASN, and/or at least two polypeptides consisting of a sequence present in RASH); at least two polypeptides consisting of a sequence present in both of RASH and RASN; and/or at least two polypeptides consisting of a sequence present in each of RASH, RASN, and RASK. In some aspects, the set comprises at least three non-identical polypeptides consisting of a sequence present in RAF1 (CRAF), BRAF, ARAF, MP2K1, MP2K2, or MK01 (e.g., at least three polypeptides consisting of a sequence present in RAF1 (CRAF), at least three polypeptides consisting of a sequence present in BRAF, at least three polypeptides consisting of a sequence present in ARAF, at least three polypeptides consisting of a sequence present in MP2K1, at least three polypeptides consisting of a sequence present in MP2K2, and/or at least three polypeptides consisting of a sequence present in MK01). In some aspects, the set comprises at least five non-identical polypeptides consisting of a sequence present in the target molecule BRAF.

In some aspects, the recombinant protein further comprises one or more additional non-identical, contiguous polypeptides consisting of a sequence present in one or more additional target molecules, wherein each of the one or more additional polypeptides comprises a cleavage site that allows separation of the polypeptide from the set upon exposure of the recombinant protein to a cleavage agent (e.g., trypsin). In some aspects, the one or more additional target molecules are components of the MAPK pathway. In other aspects, the one or more additional target molecules are not components of the MAPK pathway.

In some aspects, provided herein is a recombinant protein comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or all 42 (e.g., at least 10, at least 15, at least 20, at least 25, at least 30, at least 35, or at least 40) of a set of polypeptides having the amino acid sequences of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, and SEQ ID NO: 43, e.g., one or more of the polypeptides listed in Table 3.

TABLE 3
MAPK pathway polypeptides

| | | Uniprot | |
|---|---|---|---|
| SEQ ID NO: | Name | ID | Sequence |
| SEQ ID NO: 2 | RAF1 (CRAF) | P04049 | VVDPTPEQFQAFR |
| SEQ ID NO: 3 | RAF1 (CRAF) | P04049 | DNLAIVTQWCEGSSLYK |
| SEQ ID NO: 4 | RAF1 (CRAF) | P04049 | STSTPNVHMVSTTLPVD |
| | | | SR |
| SEQ ID NO: 5 | BRAF | P15056 | SNNIFLHEDLTVK |
| SEQ ID NO: 6 | BRAF | P15056 | LTQEHIEALLDK |
| SEQ ID NO: 7 | BRAF | P15056 | MLNVTAPTPQQLQAFK |
| SEQ ID NO: 8 | BRAF | P15056 | MLTPQQLQAFK |
| SEQ ID NO: 9 | BRAF | P15056 | IGDFGLATVK |
| SEQ ID NO: 10 | BRAFV600E | P15056 | IGDFGLATEK |
| SEQ ID NO: 11 | ARAF | P10398 | VSQPTAEQAQAFK |
| SEQ ID NO: 12 | ARAF | P10398 | IGTGSFGTVFR |
| SEQ ID NO: 13 | ARAF | P10398 | FDMVQLIDVAR |
| SEQ ID NO: 14 | polypeptide corresponding | n/a | IGDFGLATVK |
| | to a sequence present in | | |
| | each of RAF1, BRAF, and ARAF | | |
| SEQ ID NO: 15 | MP2K1 (MEK1) | Q02750 | LPSGVFSLEFQDFVNK |
| SEQ ID NO: 16 | MP2K1 (MEK1) | Q02750 | ISELGAGNGGVVFK |
| SEQ ID NO: 17 | MP2K1 (MEK1) | Q02750 | IPEQILGK |
| SEQ ID NO: 18 | MP2K2 (MEK2) | P36507 | ISELGAGNGGVVTK |
| SEQ ID NO: 19 | MP2K2 (MEK2) | P36507 | LPNGVFTPDFQEFVNK |
| SEQ ID NO: 20 | MP2K2 (MEK2) | P36507 | LNQPGTPTR |
| SEQ ID NO: 21 | MK03 (ERK1/MAPK3) | P27361 | GQPFDVGPR |
| SEQ ID NO: 22 | MK03 (ERK1/MAPK3) | P27361 | SQQLSNDHICYFLYQILR |
| SEQ ID NO: 23 | MK01 (ERK2/MAPK1) | P28482 | NYLLSLPHK |
| SEQ ID NO: 24 | MK01 (ERK2/MAPK1) | P28482 | GQVFDVGPR |
| SEQ ID NO: 25 | MK01 (ERK2/MAPK1) | P28482 | HENIIGINDIIR |
| SEQ ID NO: 26 | RASK (KRAS) | P01116 | SFEDIHHYR |
| SEQ ID NO: 27 | RASK (KRAS) | P01116 | DSEDVPMVLVGNK |
| SEQ ID NO: 28 | RASN (NRAS) | P01111 | SFADINLYR |
| SEQ ID NO: 29 | RASN (NRAS) | P01111 | QAHELAK |
| SEQ ID NO: 30 | RASH (HRAS) | P01112 | SFEDIHQYR |
| SEQ ID NO: 31 | RASH (HRAS) | P01112 | SYGIPYIETSAK |
| SEQ ID NO: 32 | polypeptide consisting of a sequence | n/a | DSDDVPMVLVGNK |
| | present in both of RASH (HRAS) and | | |
| | RASN (NRAS) | | |
| SEQ ID NO: 33 | polypeptide consisting of a sequence | n/a | SYGIPFIETSAK |
| | present in each of RASN (NRAS) and | | |
| | RASK (KRAS) | | |
| SEQ ID NO: 34 | polypeptide consisting of a sequence | n/a | QGVEDAFYTLVR |
| | present in both of RASH (HRAS) and | | |
| | RASN (NRAS) | | |
| SEQ ID NO: 35 | polypeptide consisting of a sequence | n/a | LVVVGAGGVGK |
| | present in each of RASH (HRAS), RASN | | |
| | (NRAS), and RASK (KRAS) | | |
| SEQ ID NO: 36 | polypeptide consisting of a sequence | n/a | QVVIDGETCLLDILDTA |
| | present in each of RASH (HRAS), RASN | | GQEEYSAMR |
| | (NRAS), and RASK (KRAS) | | |
| SEQ ID NO: 37 | polypeptide consisting of a sequence | n/a | QVVIDGETCLLDILDTA |
| | present in each of RASHQ61K (HRASQ61K) | | GK |
| | RASNQ61K (NRASQ61K), and RASKQ61K | | |
| | (KRASQ61K) | | |
| SEQ ID NO: 38 | polypeptide consisting of a sequence | n/a | QVVIDGETCLLDILDTA |
| | present in each of RASHQ61R | | GR |
| | (HRASQ61R), RASNO61R (NRASQ61R), and | | |
| | RASKQ61R (KRASQ61R) | | |
| SEQ ID NO: 39 | polypeptide consisting of a sequence | n/a | LVVVGAVGVGK |
| | present in each of RASHG12V (HRASG12V) | | |
| | RASNG12V (NRASG12V), and RASKG12V | | |
| | (KRASG12V) | | |
| SEQ ID NO: 40 | polypeptide consisting of a sequence | n/a | LVVVGAGDVGK |
| | present in each of RASHG13D | | |
| | (HRASG13D), RASNG13D (NRASG13D), and | | |
| | RASKG13D (KRASG13D) | | |
| SEQ ID NO: 41 | polypeptide consisting of a sequence | n/a | LVVVGACGVGK |
| | present in present in each of RASHG12C | | |
| | (HRASG120), RASNG12C (NRASG120), and | | |
| | RASKG12C (KRASG12C) | | |
| SEQ ID NO: 42 | polypeptide consisting of a sequence | n/a | LVVVGADGVGK |
| | present in each of RASHG12D | | |
| | (HRASG12D), RASNG12D (NRASG12D), and | | |
| | RASKG12D (KRASG12D) | | |
| SEQ ID NO: 43 | polypeptide consisting of a sequence | n/a | LVVVGASGVGK |
| | present in each of RASHG12S | | |
| | (HRASG12S), RASNG12S (NRASG125), and | | |
| | RASKG12S (KRASG12S) | | |

In some aspects, provided herein is a recombinant protein comprising a set of polypeptides having the amino acid sequences of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, and SEQ ID NO: 43.

In some aspects, the recombinant protein further comprises an N-terminal sequence comprising methionine and a cleavage site (e.g., a trypsin cleavage site) between the N-terminal sequence and the set of polypeptides that allows separation of the N-terminal sequence from the set of polypeptides upon exposure of the recombinant protein to a cleavage agent (e.g., trypsin). In some aspects, the N-terminal sequence has the amino acid sequence of SEQ ID NO: 88. In some aspects, the N-terminal sequence further comprises a tag. In some aspects, the tag is a polyhistidine tag. In some aspects, the tag is a FLAG tag (e.g., a FLAG tag comprising the amino acid sequence of SEQ ID NO: 90), a HA tag (e.g., a HA tag comprising the amino acid sequence of SEQ ID NO: 91), or a V5 tag (e.g., a V5 tag comprising the amino acid sequence of SEQ ID NO: 92). In some aspects, the tag is a tandem tag, e.g., a 2×, 3×, 4×, 5×, 6×, 7×, or 8×FLAG tag, His tag, or HA tag. In some aspects, the tandem tag is a 3×tag. In some aspects, the tandem tag is heterogeneous, e.g., comprises two or more of a FLAG tag, a HA tag, or V5 tag. In some aspects, the tandem tag is a His-FLAG tag.

In some aspects, the recombinant protein further comprises a C-terminal sequence comprising a tag and a cleavage site (e.g., a trypsin cleavage site) between the C-terminal sequence and the set of polypeptides that allows separation of the C-terminal sequence from the set of polypeptides upon exposure of the recombinant protein to a cleavage agent (e.g., trypsin). In some aspects, the tag is a polyhistidine tag. In some aspects, the C-terminal sequence has the amino acid sequence of SEQ ID NO: 89. In some aspects, the tag is a FLAG tag (e.g., a FLAG tag comprising the amino acid sequence of SEQ ID NO: 90), a HA tag (e.g., a HA tag comprising the amino acid sequence of SEQ ID NO: 91), or a V5 tag (e.g., a V5 tag comprising the amino acid sequence of SEQ ID NO: 92). In some aspects, the tag is a tandem tag, e.g., a 2×, 3×, 4×, 5×, 6×, 7×, or 8×FLAG tag, His tag, or HA tag. In some aspects, the tandem tag is a 3×tag. In some aspects, the tandem tag is heterogeneous, e.g., comprises two or more of a FLAG tag, a HA tag, or V5 tag. In some aspects, the tandem tag is a His-FLAG tag.

In some aspects, the recombinant protein comprises the amino acid sequence of SEQ ID NO: 1.

In some aspects, provided herein is a recombinant protein consisting of the amino acid sequence of SEQ ID NO: 1.

In some aspects, each polypeptide of the set of non-identical, contiguous polypeptides comprised by the recombinant protein comprises a label. In some aspects, the label is an isotopic label. In some aspects, the isotopic label is heavy arginine. In some aspects, the heavy arginine is ¹³C₁-arginine ¹³C₁-arginine (R1); ¹³C₂-arginine (R2); ¹⁵N₄-arginine (R4); ¹³C₆-arginine (R6); ²H₇-arginine (R7); ¹³C₆, ¹⁵N₄-arginine (R10); ²H₇, ¹⁵N₄-arginine (R11), or ¹³C₆, ²H₇, ¹⁵N₄-arginine (R17). Representative heavy arginine species are provided in Table 1. In some aspects, the isotopic label is heavy lysine. In some aspects, the heavy lysine is ¹³C₁-lysine (K1); ¹⁵N₂-lysine (K2); ²H₄-lysine (K4); ¹³C₆-lysine (K6); ¹³C₆, ¹⁵N₂-lysine (K8); ²H₈-lysine (K8); ²H₉-lysine (K9); ²H₉, ¹⁵N₂-lysine (K11); or ¹³C₆; ²H₉, ¹⁵N₂-lysine (K17). Representative heavy lysine species are provided in Table 2. Further examples of lysine isotopic labels include the Thermo Fisher NEUCODE™ lysines K521, K440, 390, 642, 192, and 202 (13C, 2H, 15N).

In some aspects, the label is a chemical label. In some aspects, the chemical label is a tandem mass tag (TMT), an iTRAQ, a label produced by reductive methylation/dimethylation, or a label produced by acetylation (e.g., acetic anhydride). In some aspects, the label (e.g., chemical label) is an isobaric label.

In some aspects, the recombinant protein is at least 98% labeled. In some aspects, the recombinant protein is at least 99% labeled. In some aspects, the recombinant protein is 100% labeled.

In some aspects, provided herein is a nucleic acid encoding any one of the recombinant proteins described herein.

### C. Recombinant Proteins Useful in Assessment of PI3K Pathway Components

Provided herein are recombinant proteins comprising a set of non-identical, contiguous polypeptides consisting of sequences present in one or more members of the phosphoinositide 3-kinase (PI3K) pathway, or mutant or variant forms thereof, e.g., sequences present in one or more of P85A, P85B, PK3CA, PK3CA^{E545K}, PK3CA^{H1047K}, PK3CD, PK3CB, ERBB2 (HER2), EGFR, RRAS2, and P55G.

In some aspects, provided herein is a recombinant protein comprising a set of non-identical, contiguous polypeptides, the set comprising one or more of a polypeptide consisting of a sequence present in P85A; a polypeptide consisting of a sequence present in P85B; a polypeptide consisting of a sequence present in PK3CA; a polypeptide consisting of a sequence present in PK3CA^{E545K}; a polypeptide consisting of a sequence present in PK3CA^{H1047K}; a polypeptide consisting of a sequence present in PK3CD; a polypeptide consisting of a sequence present in PK3CB; a polypeptide consisting of a sequence present in ERBB2; a polypeptide consisting of a sequence present in EGFR; a polypeptide consisting of a sequence present in RRAS2; and a polypeptide consisting of a sequence present in P55G, e.g., comprising at least 1, 2, 3, 4, 5, 6, 7, 8, 9, 10, or all 11 polypeptides listed above, wherein the recombinant protein comprises a cleavage site (e.g., a trypsin cleavage site) between each polypeptide of the set that allows separation of each polypeptide upon exposure of the recombinant protein to a cleavage agent (e.g., trypsin).

In some aspects, provided herein is a recombinant protein comprising a set of non-identical, contiguous polypeptides, the set comprising a polypeptide consisting of a sequence present in P85A; a polypeptide consisting of a sequence present in P85B; a polypeptide consisting of a sequence present in PK3CA; a polypeptide consisting of a sequence present in PK3CA^{E545K}; a polypeptide consisting of a sequence present in PK3CA^{H1047K}; a polypeptide consisting of a sequence present in PK3CD; a polypeptide consisting of a sequence present in PK3CB; a polypeptide consisting of a sequence present in ERBB2; a polypeptide consisting of a sequence present in EGFR; a polypeptide consisting of a sequence present in RRAS2; and a polypeptide consisting of a sequence present in P55G, wherein the recombinant protein comprises a cleavage site (e.g., a trypsin cleavage site) between each polypeptide of the set that allows separation of each polypeptide upon exposure of the recombinant protein to a cleavage agent (e.g., trypsin). In some aspects, the recombinant protein further comprises a sequence present in G3P and/or further comprises a sequence present in ACTA.

In some aspects, each of the polypeptides is between 6 and 25 amino acid residues in length, e.g., is 6, 7, 8, 9, 10, 11, 12, 13, 14, 15, 16, 17, 18, 19, 20, 21, 22, 23, 24, or 25 amino acid residues in length.

In some aspects, the polypeptide consisting of a sequence present in PK3CA^{E545K} has the amino acid sequence of SEQ ID NO: 56 and/or the polypeptide consisting of a sequence present in PK3CA^{H1047K} has the amino acid sequence of SEQ ID NO: 58 or SEQ ID NO: 59.

In some aspects, the set of non-identical, contiguous polypeptides comprised by the recombinant protein includes at least two, at least three, or at least four non-identical polypeptides consisting of a sequence present in a target molecule. In some aspects, the set comprises at least two non-identical polypeptides consisting of a sequence present in P85A, P85B, PK3CA, PK3CA^{E545K}, PK3CA^{H1047K}, PK3CD, PK3CB, ERBB2, EGFR, RRAS2, or P55G, e.g., at least two polypeptides consisting of a sequence present in P85A, at least two polypeptides consisting of a sequence present in P85B, at least two polypeptides consisting of a sequence present in PK3CA, at least two polypeptides consisting of a sequence present in PK3CA^{E545K}, at least two polypeptides consisting of a sequence present in PK3CA^{H1047K}, at least two polypeptides consisting of a sequence present in PK3CD, at least two polypeptides consisting of a sequence present in PK3CB, at least two polypeptides consisting of a sequence present in ERBB2, at least two polypeptides consisting of a sequence present in EGFR, at least two polypeptides consisting of a sequence present in RRAS2, or at least two polypeptides consisting of a sequence present in P55G.

In some aspects, the recombinant protein further comprises a polypeptide consisting of a sequence present in a control protein. In some aspects, the control protein is G3P or ACTA. In some aspects, the recombinant protein comprises a polypeptide consisting of a sequence present in G3P and a polypeptide consisting of a sequence present in ACTA. In some aspects, the set comprises at least two polypeptides consisting of a sequence present in G3P or ACTA, e.g., at least two polypeptides consisting of a sequence present in G3P or at least two polypeptides consisting of a sequence present in ACTA.

In some aspects, the recombinant protein further comprises one or more additional non-identical, contiguous polypeptides consisting of a sequence present in one or more additional target molecules, wherein each of the one or more additional polypeptides comprises a cleavage site that allows separation of the polypeptide from the set upon exposure of the recombinant protein to a cleavage agent (e.g., trypsin). In some aspects, the one or more additional target molecules are components of the PI3K pathway. In other aspects, the one or more additional target molecules are not components of the PI3K pathway.

In some aspects, provided herein is a recombinant protein comprising a set of polypeptides having the amino acid sequences of SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 82, SEQ ID NO: 83, SEQ ID NO: 84, SEQ ID NO: 85, SEQ ID NO: 86, and SEQ ID NO: 87, e.g., one or more of the polypeptides listed in Table 4. In some embodiments, the recombinant protein further comprises polypeptides having the amino acid sequences of SEQ ID NO: 76, SEQ ID NO: 77, SEQ ID NO: 78, SEQ ID NO: 79, SEQ ID NO: 80, and/or SEQ ID NO: 81. In some embodiments, the recombinant protein further comprises polypeptides having the amino acid sequences of SEQ ID NO: 76, SEQ ID NO: 77, SEQ ID NO: 78, SEQ ID NO: 79, SEQ ID NO: 80, and SEQ ID NO: 81, e.g., one or more of the polypeptides listed in Table 5.

In some aspects, provided herein is a recombinant protein comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 or all 43 (e.g., at least 10, at least 15, at least 20, at least 25, at least 30, at least 35, or at least 40) of a set of polypeptides having the amino acid sequences of SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, and SEQ ID NO: 87, e.g., one or more of the polypeptides listed in Table 4 or Table 5.

TABLE 4
PI3K pathway polypeptides

| SEQ ID NO: | Name | Uniprot ID | Sequence |
|---|---|---|---|
| SEQ ID NO: 45 | P85A | P27986 | FSAASSDNTENLIK |
| SEQ ID NO: 46 | P85A | P27986 | TQSSSNLAELR |
| SEQ ID NO: 47 | P85A | P27986 | NESLAQYNPK |
| SEQ ID NO: 48 | P85B | O00459 | AALQALGVAEGGER |
| SEQ ID NO: 49 | P85B | O00459 | ALGATFGPLLLR |
| SEQ ID NO: 50 | P85B | O00459 | APGPGPPPAAR |
| SEQ ID NO: 51 | PK3CA | P42336 | EAGFSYSHAGLSNR |
| SEQ ID NO: 52 | PK3CA | P42336 | YEQYLDNLLVR |
| SEQ ID NO: 53 | PK3CA | P42336 | EATLITIK |
| SEQ ID NO: 54 | PK3CA | P42336 | DLNSPHSR |
| SEQ ID NO: 55 | PK3CA | P42336 | DPLSEITEQEK |
| SEQ ID NO: 56 | PK3CAE545K | P42336 | DPLSEITK |
| SEQ ID NO: 57 | PK3CA | P42336 | QMNDAHHGGWTTK |
| SEQ ID NO: 58 | PK3CAH1047K | P42336 | QMNDAR |
| SEQ ID NO: 59 | PK3CAH1047K | P42336 | HGGWTTK |
| SEQ ID NO: 60 | PK3CD | O00329 | TGLIEVVLR |
| SEQ ID NO: 61 | PK3CD | O00329 | GELLNPTGTVR |
| SEQ ID NO: 62 | PK3CD | O00329 | IELIQGSK |
| SEQ ID NO: 63 | PK3CD | O00329 | HEVQEHFPEALAR |
| SEQ ID NO: 64 | PK3CD | O00329 | NANLSTIK |
| SEQ ID NO: 65 | PK3CB | P42338 | EIFPQSLPK |
| SEQ ID NO: 66 | PK3CB | P42338 | AAEIASSDSANVSSR |
| SEQ ID NO: 67 | PK3CB | P42338 | EATISYIK |
| SEQ ID NO: 68 | PK3CB | P42338 | VFGEDSVGVIFK |
| SEQ ID NO: 69 | PK3CB | P42338 | EYNSGDDLDR |
| SEQ ID NO: 70 | ERBB2 (HER2) | P04626 | VLQGLPR |
| SEQ ID NO: 71 | ERBB2 (HER2) | P04626 | GLQSLPTHDPSPLQR |
| SEQ ID NO: 72 | ERBB2 (HER2) | P04626 | FVVIQNEDLGPASPLDSTFYR |
| SEQ ID NO: 73 | EGFR | P00533 | TDLHAFENLEIIR |
| SEQ ID NO: 74 | EGFR | P00533 | IPLENLQIIR |
| SEQ ID NO: 75 | EGFR | P00533 | YLVIQGDER |
| SEQ ID NO: 82 | RRAS2 | P62070 | QVTQEEGQQLAR |
| SEQ ID NO: 83 | RRAS2 | P62070 | GSFEEIYK |
| SEQ ID NO: 84 | RRAS2 | P62070 | LVVVGGGGVGK |
| SEQ ID NO: 85 | P55G | Q92569 | VQAEDLLYGKPDGAFLIR |
| SEQ ID NO: 86 | P55G | Q92569 | HCVIYSTAR |
| SEQ ID NO: 87 | P55G | Q92569 | LQEYHSQYQEK |

TABLE 5
Control polypeptides

| SEQ ID NO: | Name | Uniprot ID | Sequence |
|---|---|---|---|
| SEQ ID NO: 76 | G3P | P04406 | LVINGNPITIFQER |
| SEQ ID NO: 77 | G3P | P04406 | GALQNIIPASTGAAK |
| SEQ ID NO: 78 | G3P | P04406 | VGVNGFGR |
| SEQ ID NO: 79 | ACTA | P62736 | AGFAGDDAPR |
| SEQ ID NO: 80 | ACTA | P62736 | AVFPSIVGRPR |
| SEQ ID NO: 81 | ACTA | P62736 | SYELPDGQVITIGNER |

In some aspects, the recombinant protein further comprises an N-terminal sequence comprising methionine and a cleavage site (e.g., a trypsin cleavage site) between the N-terminal sequence and the set of polypeptides that allows separation of the N-terminal sequence from the set of polypeptides upon exposure of the recombinant protein to a cleavage agent (e.g., trypsin). In some aspects, the N-terminal sequence has the amino acid sequence of SEQ ID NO: 88.

In some aspects, the recombinant protein further comprises a C-terminal sequence comprising a tag and a cleavage site (e.g., a trypsin cleavage site) between the C-terminal sequence and the set of polypeptides that allows separation of the C-terminal sequence from the set of polypeptides upon exposure of the recombinant protein to a cleavage agent (e.g., trypsin). In some aspects, the tag is a polyhistidine tag. In some aspects, the C-terminal sequence has the amino acid sequence of SEQ ID NO: 89. In some aspects, the tag is a FLAG tag (e.g., a FLAG tag comprising the amino acid sequence of SEQ ID NO: 90), a HA tag (e.g., a HA tag comprising the amino acid sequence of SEQ ID NO: 91), or a V5 tag (e.g., a V5 tag comprising the amino acid sequence of SEQ ID NO: 92). In some aspects, the tag is a tandem tag, e.g., a 2×, 3×, 4×, 5×, 6×, 7×, or 8×FLAG tag, His tag, or HA tag. In some aspects, the tandem tag is a 3×tag. In some aspects, the tandem tag is heterogeneous, e.g., comprises two or more of a FLAG tag, a HA tag, or V5 tag. In some aspects, the tandem tag is a His-FLAG tag.

In some aspects, the recombinant protein comprises the amino acid sequence of SEQ ID NO: 44.

In some aspects, provided herein is a recombinant protein consisting of the amino acid sequence of SEQ ID NO: 44.

In some aspects, each polypeptide of the set of non-identical, contiguous polypeptides comprised by the recombinant protein comprises a label. In some aspects, the label is an isotopic label. In some aspects, the isotopic label is heavy arginine. In some aspects, the heavy arginine is ¹³C₁-arginine ¹³C₁-arginine (R1); ¹³C₂-arginine (R2); ¹⁵N₄-arginine (R4); ¹³C₆-arginine (R6); ²H₇-arginine (R7); ¹³C₆, ¹⁵N₄-arginine (R10); ²H₇, ¹⁵N₄-arginine (R11), or ¹³C₆, ²H₇, ¹⁵N₄-arginine (R17). Representative heavy arginine species are provided in Table 1. In some aspects, the isotopic label is heavy lysine. In some aspects, the heavy lysine is ¹³C₁-lysine (K1); ¹⁵N₂-lysine (K2); ²H₄-lysine (K4); ¹³C₆-lysine (K6); ¹³C₆, ¹⁵N₂-lysine (K8); ²H₈-lysine (K8); ²H₉-lysine (K9); ²H₉, ¹⁵N₂-lysine (K11); or ¹³C₆; ²H₉, ¹⁵N₂-lysine (K17). Representative heavy lysine species are provided in Table 2. Further examples of lysine isotopic labels include the Thermo Fisher NEUCODE™ lysines K521, K440, 390, 642, 192, and 202 (13C, 2H, 15N).

In some aspects, the label is a chemical label. In some aspects, the chemical label is a tandem mass tag (TMT), an iTRAQ, a label produced by reductive methylation/dimethylation, or a label produced by acetylation (e.g., acetic anhydride). In some aspects, the label (e.g., chemical label) is an isobaric label.

In some aspects, the recombinant protein is at least 98% labeled. In some aspects, the recombinant protein is at least 99% labeled. In some aspects, the recombinant protein is 100% labeled.

In some aspects, provided herein is a nucleic acid encoding any one of the recombinant proteins described herein.

### III. Methods for Determining Protein Levels

### A. Methods for Determining Protein Levels of MAPK Pathway Components

In some aspects, provided herein is a method for determining a protein level (e.g., an absolute protein level) in a sample from a subject of one or more of RAF1 (CRAF), ARAF, BRAF, BRAF^{V600E}, RASH (HRAS), RASH^{Q61K} (HRAS^{Q61K}), RASH^{Q61R} (HRAS^{Q61R}), RASH^{G12V}(HRAS^{G12V}), RASH^{G13D} (HRAS^{G13D}), RASH^{G12C} (HRAS^{G12C}), RASH^{G12D} (HRAS^{G12D}), RASH^{G12S} (HRAS^{G12S}), RASK (KRAS), RASK^{Q61K} (KRAS^{Q61K}), RASK^{Q61R} (KRAS^{Q61R}), RASK^{G12V}(KRAS^{G12V}), RASK^{G13D}(KRAS^{G13D}), RASK^{G12C} (KRAS^{G12C}), RASK^{G12D} (KRAS^{G12D}), RASK^{G12S}(KRAS^{G12S}), MP2K1 (MEK1), MP2K2 (MEK2), MK03 (ERK1/MAPK3), MK01 (ERK2/MAPK1), RASN (NRAS), RASN^{Q61K}(NRAS^{Q61K}), RASN^{Q61R} (NRAS^{Q61R}), RASN^{G12V} (NRAS^{G12V}), RASN^{G13D} (NRAS^{G13D}), RASN^{G12C} (NRAS^{G12C}), RASN^{G12D}(NRAS^{G12D}), RASN^{G12S} (NRAS^{G12S}); the method comprising (a) adding to the sample an amount of a recombinant protein described in Section IIB herein, wherein the recombinant protein comprises at least one polypeptide consisting of a sequence present in the protein for which the level is determined; (b) exposing the sample following step (a) to a cleavage agent (e.g., trypsin), whereby the recombinant protein and proteins from the sample are cleaved, thereby generating an equimolar set of internal standard polypeptides, the set comprising at least one polypeptide consisting of a sequence present in each of the proteins for which the level is determined; (c) measuring a level of one or more internal standard polypeptides and a level of one or more corresponding polypeptides from the sample (e.g., measuring a level of the internal standard polypeptide consisting of a sequence present in RAF1 and measuring a level of RAF1 from the sample); and (d) comparing the level of the one or more internal standard polypeptides and the level of the one or more corresponding polypeptides from the sample, thereby determining a protein level of one or more of: RAF1, BRAF, BRAF^{V600E}, ARAF, MP2K1, MP2K2, MK03, MK01, RASK, RASN, RASH; RASH and RASN; RASN and RASK; RASH, RASN, and RASK; RASH^{Q61K}, RASN^{Q61K}, and RASK^{Q61K}; RASH^{Q61R}, RASN^{Q61R}, and RASK^{Q61R}; RASH^{G12V}, RASN^{G12V}, and RASK^{G12V}; RASH^{G13D}, RASN^{G13D}, and RASK^{G13D}; RASH^{G12C}, RASN^{G12C}, and RASK^{G12C}; RASH^{G12D}, RASN^{G12D}, and RASK^{G12D}; and RASH^{G12S}, RASN^{G12S}, and RASK^{G12S} in the sample.

In some aspects, provided herein is a method for determining a protein level in a sample from a subject of one or more of RAF1, BRAF, BRAF^{V600E}, ARAF, MP2K1, MP2K2, MK03, MK01, RASK, RASN, RASH; RASH and RASN; RASN and RASK; RASH, RASN, and RASK; RASH^{Q61K}, RASN^{Q61K}, and RASK^{Q61K}; RASH^{Q61R}, RASN^{Q61R}, and RASK^{Q61R}; RASH^{G12V}, RASN^{G12V}, and RASK^{G12V}; RASH^{G13D}, RASN^{G13D}, and RASK^{G13D}; RASH^{G12C}, RASN^{G12C}, and RASK^{G12C}; RASH^{G12D}, RASN^{G12D}, and RASK^{G12D}; and RASH^{G12S}, RASN^{G12S}, and RASK^{G12S}; the method comprising (a) adding to the sample an amount of a recombinant protein described in Section IIB herein, wherein the recombinant protein comprises at least one polypeptide consisting of a sequence present in each of RAF1, BRAF, BRAF^{V600E}, ARAF, MP2K1, MP2K2, MK03, MK01, RASK, RASN, RASH; RASH and RASN; RASN and RASK; RASH, RASN, and RASK; RASH^{Q61K}, RASN^{Q61K}, and RASK^{Q61K}; RASH^{Q61R}, RASN^{Q61R}, and RASK^{Q61R}; RASH^{G12V}, RASN^{G12V}, and RASK^{G12V}; RASH^{G13D}, RASN^{G13D}, and RASK^{G13D}; RASH^{G12C}, RASN^{G12C}, and RASK^{G12C}; RASH^{G12D}, RASN^{G12D}, and RASK^{G12D}; and RASH^{G12S}, RASN^{G12S}, and RASK^{G12S}; (b) exposing the sample following step (a) to a cleavage agent (e.g., trypsin), whereby the recombinant protein and proteins from the sample are cleaved, thereby generating an equimolar set of internal standard polypeptides, the set comprising one or more of a polypeptide consisting of a sequence present in RAF1; a polypeptide consisting of a sequence present in BRAF; a polypeptide consisting of a sequence present in BRAF^{V600E}a polypeptide consisting of a sequence present in ARAF; a polypeptide consisting of a sequence present in MP2K1; a polypeptide consisting of a sequence present in MP2K2; a polypeptide consisting of a sequence present in MK03; a polypeptide consisting of a sequence present in MK01; a polypeptide consisting of a sequence present in RASK; a polypeptide consisting of a sequence present in RASN; a polypeptide consisting of a sequence present in RASH; a polypeptide consisting of a sequence present in each of RAF1, BRAF, and ARAF; a polypeptide consisting of a sequence present in both of RASH and RASN; a polypeptide consisting of a sequence present in both of RASN and RASK; a polypeptide consisting of a sequence present in each of RASH, RASN, and RASK; a polypeptide consisting of a sequence present in each of RASH^{Q61K}, RASN^{Q61K}, and RASK^{Q61K}; a polypeptide consisting of a sequence present in each of RASH^{Q61R}, RASN^{Q61R}, and RASK^{Q61R}; a polypeptide consisting of a sequence present in each of RASH^{G12V}, RASN^{G12V}, and RASK^{G12V}; a polypeptide consisting of a sequence present in each of RASH^{G13D}, RASN^{G13D}, and RASK^{G13D}; a polypeptide consisting of a sequence present in each of RASH^{G12C}, RASN^{G12C}, and RASK^{G12C}; a polypeptide consisting of a sequence present in each of RASH^{G12D}, RASN^{G12D}, and RASK^{G12D}; and a polypeptide consisting of a sequence present in each of RASH^{G12S}, RASN^{G12S}, and RASK^{G12S}; (c) measuring a level of one or more internal standard polypeptides and a level of one or more corresponding polypeptides from the sample; and (d) comparing the level of the one or more internal standard polypeptides and the level of the one or more corresponding polypeptides from the sample, thereby determining a protein level of one or more of RAF1, BRAF, BRAF^{V600E}, ARAF, MP2K1, MP2K2, MK03, MK01, RASK, RASN, RASH; RASH and RASN; RASN and RASK; RASH, RASN, and RASK; RASH^{Q61K}, RASN^{Q61K}, and RASK^{Q61K}; RASH^{Q61R}, RASN^{Q61R}, and RASK^{Q61R}; RASH^{G12V}, RASN^{G12V}, and RASK^{G12V}; RASH^{G13D}, RASN^{G13D}, and RASK^{G13D}; RASH^{G12C}, RASN^{G12C}, and RASK^{G12C}; RASH^{G12D}, RASN^{G12D}, and RASK^{G12D}; and RASH^{G12S}, RASN^{G12S}, and RASK^{G12S} in the sample.

In some aspects, the method comprises determining a protein level (e.g., an absolute protein level) of one or more of RASH, RASN, RASK, ARAF, BRAF, and RAF1 in the sample. In some aspects, the method comprises determining a protein level of each of RASH, RASN, RASK, ARAF, BRAF, and RAF1 in the sample.

In some aspects, the protein level is a relative protein level. In some aspects, the protein level is an absolute protein level.

In some aspects, the method is performed for at least two samples from the subject (e.g., two, three, four, five, or more than five samples from the subject). In some aspects, the at least two samples (e.g., two, three, four, five, or more than five samples) are from at least two different time points (e.g., two, three, four, five, or more than five time points). In some aspects, the at least two different time points (e.g., two, three, four, five, or more than five time points) include at least one time point before administration of an agent (e.g., a therapeutic agent) to the subject and at least one timepoint after administration of the agent (e.g., therapeutic agent) to the subject.

In some aspects, the measuring of step (c) comprises mass spectrometry (MS). In some aspects, the MS is parallel reaction monitoring MS (PRM-MS).

In some aspects, the sample is a human sample. In some aspects, the sample is a tumor sample. In some aspects, the sample is a lysate.

In some aspects, the sample is an immunoprecipitate of a target protein. In some aspects, the sample is an immunoprecipitate of a MAPK pathway protein or a variant or mutant form thereof, e.g., an immunoprecipitate of RAF1, BRAF, BRAF^{V600E}, ARAF, MP2K1, MP2K2, MK03, MK01, RASK, RASN, RASH; RASH and RASN; RASN and RASK; RASH, RASN, and RASK; RASH^{Q61K}, RASN^{Q61K}, and RASK^{Q61K}; RASH^{Q61R}, RASN^{Q61R}, and RASK^{Q61R}; RASH^{G12V}, RASN^{G12V}, and RASK^{G12V}; RASH^{G13D}, RASN^{G13D}, and RASK^{G13D}; RASH^{G12C}, RASN^{G12C}, and RASK^{G12C}; RASH^{G12D}, RASN^{G12D}, and RASK^{G12D}; and RASH^{G12S}, RASN^{G12S}, or RASK^{G12S}.

In some aspects, the method comprises determining the ratio of the target protein to one or more of RAF1, BRAF, BRAF^{V600E}, ARAF, MP2K1, MP2K2, MK03, MK01, RASK, RASN, RASH; RASH and RASN; RASN and RASK; RASH, RASN, and RASK; RASH^{Q61K}, RASN^{Q61K}, and RASK^{Q61K}; RASH^{Q61R}, RASN^{Q61R}, and RASK^{Q61R}; RASH^{G12V}, RASN^{G12V}, and RASK^{G12V}; RASH^{G13D}, RASN^{G13D}, and RASK^{G13D}; RASH^{G12C}, RASN^{G12C}, and RASK^{G12C}; RASH^{G12D}, RASN^{G12D}, and RASK^{G12D}; and RASH^{G12S}, RASN^{G12S}, and RASK^{G12S} In some aspects, the recombinant protein used in the method comprises a set of polypeptides having the amino acid sequences of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, and SEQ ID NO: 43.

In some aspects, the recombinant protein used in the method comprises the amino acid sequence of SEQ ID NO: 1.

In some aspects, the recombinant protein used in the method consists of the amino acid sequence of SEQ ID NO: 1.

### B. Methods for Determining Protein Levels of PI3K Pathway Components

In some aspects, provided herein is a method for determining a protein level (e.g., an absolute protein level) in a sample from a subject of one or more of P85A, P85B, PK3CA (also called PIK3CA and p¹10-alpha), PK3CA^{E545K}, PK3CA^{H1047K}, PK3CD, PK3CB, ERBB2 (HER2), EGFR, RRAS2, and P55G, the method comprising (a) adding to the sample an amount of a recombinant protein described in Section 11C herein, wherein the recombinant protein comprises at least one polypeptide consisting of a sequence present in the protein for which the level is determined; (b) exposing the sample following step (a) to a cleavage agent (e.g., trypsin), whereby the recombinant protein and proteins from the sample are cleaved, thereby generating an equimolar set of internal standard polypeptides, the set comprising at least one polypeptide consisting of a sequence present in each of the proteins for which the level is determined; (c) measuring a level of one or more internal standard polypeptides and a level of one or more corresponding polypeptides from the sample (e.g., measuring a level of the internal standard polypeptide consisting of a sequence present in P85A and measuring a level of P85A from the sample); and (d) comparing the level of the one or more internal standard polypeptides and the level of the one or more corresponding polypeptides from the sample, thereby determining a protein level of one or more of P85A, P85B, PK3CA, PK3CA^{E545K}, PK3CA^{H1047K}, PK3CD, PK3CB, ERBB2, EGFR, RRAS2, and P55G in the sample.

In some aspects, provided herein is a method for determining a protein level in a sample from a subject of one or more of P85A, P85B, PK3CA, PK3CA^{E545K}, PK3CA^{H1047K}, PK3CD, PK3CB, ERBB2, EGFR, RRAS2, and P55G, the method comprising (a) adding to the sample an amount of a recombinant protein described in Section IIC herein, wherein the recombinant protein comprises at least one polypeptide consisting of a sequence present in each of P85A, P85B, PK3CA, PK3CA^{E545K}, PK3CA^{H1047K}PK3CD, PK3CB, ERBB2, EGFR, RRAS2, and P55G; (b) exposing the sample following step (a) to a cleavage agent (e.g., trypsin), whereby the recombinant protein and proteins from the sample are cleaved, thereby generating an equimolar set of internal standard polypeptides, the set comprising one or more of a polypeptide consisting of a sequence present in P85A; a polypeptide consisting of a sequence present in P85B; a polypeptide consisting of a sequence present in PK3CA; a polypeptide consisting of a sequence present in PK3CA^{E545K}; a polypeptide consisting of a sequence present in PK3CA^{H1047K}; a polypeptide consisting of a sequence present in PK3CD; a polypeptide consisting of a sequence present in PK3CB; a polypeptide consisting of a sequence present in ERBB2; a polypeptide consisting of a sequence present in EGFR; a polypeptide consisting of a sequence present in RRAS2; and a polypeptide consisting of a sequence present in P55G; (c) measuring a level of one or more internal standard polypeptides and a level of one or more corresponding polypeptides from the sample; and (d) comparing the level of the one or more internal standard polypeptides and the level of the one or more corresponding polypeptides from the sample, thereby determining a protein level of one or more of P85A, P85B, PK3CA, PK3CA^{E545K}, PK3CA^{H1047K}, PK3CD, PK3CB, ERBB2, EGFR, RRAS2, and P55G in the sample.

In some aspects, the method further comprises determining a protein level in the sample from the subject of G3P and/or ACTA, wherein the recombinant protein of step (a) comprises a polypeptide consisting of a sequence present in G3P and/or a polypeptide consisting of a sequence present in ACTA, and the set of internal standard polypeptides of step (b) comprises a polypeptide consisting of a sequence present in G3P and/or a polypeptide consisting of a sequence present in ACTA. In some aspects, the method comprises determining a protein level in the sample from the subject of both G3P and ACTA.

In some aspects, the protein level is a relative protein level. In some aspects, the protein level is an absolute protein level.

In some aspects, the method is performed for at least two samples from the subject (e.g., two, three, four, five, or more than five samples from the subject). In some aspects, the at least two samples (e.g., two, three, four, five, or more than five samples) are from at least two different time points (e.g., two, three, four, five, or more than five time points). In some aspects, the at least two different time points (e.g., two, three, four, five, or more than five time points) include at least one time point before administration of an agent (e.g., a therapeutic agent) to the subject and at least one timepoint after administration of the agent (e.g., therapeutic agent) to the subject.

In some aspects, the measuring of step (c) comprises mass spectrometry (MS). In some aspects, the MS is parallel reaction monitoring MS (PRM-MS).

In some aspects, the sample is a human sample. In some aspects, the sample is a tumor sample. In some aspects, the sample is a lysate.

In some aspects, the sample is an immunoprecipitate of a target protein. In some aspects, the sample is an immunoprecipitate of a PI3K pathway protein or a variant or mutant form thereof, e.g., an immunoprecipitate of P85A, P85B, PK3CA, PK3CA^{E545K}, PK3CA^{H1047K}, PK3CD, PK3CB, ERBB2, EGFR, RRAS2, or P55G.

In some aspects, the method comprises determining the ratio of the target protein to one or more of P85A, P85B, PK3CA, PK3CA^{E545K}, PK3CA^{H1047K}, PK3CD, PK3CB, ERBB2, EGFR, G3P, ACTA, RRAS2, and P55G.

In some aspects, a single species of recombinant protein is added to the sample. In other aspects, at least two, at least three, at least four, at least five, or more than five species of recombinant proteins are added to the sample. In some aspects, a recombinant protein useful for assessing levels of one or more members of the MAPK pathway (e.g., a recombinant protein described in Section IIIB herein) and an additional recombinant protein comprising a different set of polypeptides are added to the sample.

In some aspects, a recombinant protein useful for assessing levels of one or more members of the PI3K pathway (e.g., a recombinant protein described in Section IIIC herein) and an additional recombinant protein comprising a different set of polypeptides are added to the sample. In some aspects, a recombinant protein useful for assessing levels of one or more members of the MAPK pathway and a recombinant protein useful for assessing levels of one or more members of the PI3K pathway, are added to the sample, e.g., a recombinant protein consisting of the amino acid sequence of SEQ ID NO: 1 and a recombinant protein consisting of the amino acid sequence of SEQ ID NO: 44 are added to the sample. The two or more species of recombinant protein may comprise the same label, or may comprise different labels.

In some aspects, the recombinant protein used in the method comprises a set of polypeptides having the amino acid sequences of SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, and SEQ ID NO: 87, e.g., one or more of the polypeptides listed in Table 4 or Table 5.

In some aspects, the recombinant protein used in the method comprises the amino acid sequence of SEQ ID NO: 44.

In some aspects, the recombinant protein used in the method consists of the amino acid sequence of SEQ ID NO: 44.

### IV. EXAMPLES

The following are examples of methods, uses, and compositions of the invention. It is understood that various other aspects may be practiced, given the general description provided above, and the examples are not intended to limit the scope of the claims.

### Example 1. Assessment of the Functional Role of RAF Isoforms in RASK (KRAS) and RASN (NRAS) Mutant Tumors

a. Analysis of CERES Scores

Approximately 30% of all tumors harbor mutations in the RASK (KRAS) gene, and there are currently no approved targeted therapies for RASK (KRAS) mutant cancers. In RASK (KRAS) mutant lung tumors, depletion of RAF1 (CRAF) and not BRAF prevents tumor growth despite ERK signaling remaining intact. It has previously been demonstrated that single agent pan-RAF inhibitors exhibit limited efficacy in RASK (KRAS) mutant tumor cell lines (Yen et al., Cancer Cell, 34: 611-625, 2018; Whittaker et al., Mol Cancer Ther, 14: 2700-2711, 2015). To assess the functional role of each RAF isoform in RASK (KRAS) and RASN (NRAS) mutant tumors, we performed a comparative analysis of the Project Achilles dataset (BROAD, the Cancer Dependency Map (DepMap)) (Tsherniak et al., Cell, 170: 564-576 e516, 2017; Yu et al., Nat Biotechnol, 34: 419-423, 2016).

The DepMap dataset was generated by a CRISPR screen in which about 30,000 genes were knocked out in multiple cancer cell lines and viability of the cell lines was assessed. A viability score (CERES score) of <−1 upon loss of a gene indicates that the cell line is dependent on that gene for growth. Thus, a CERES score refers to a cumulative dependency of a cancer cell line on a single gene that is required for its viability and growth.

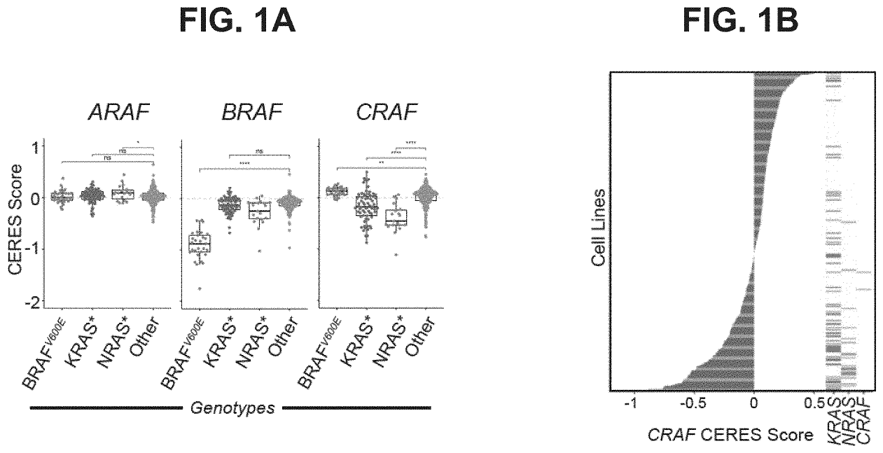

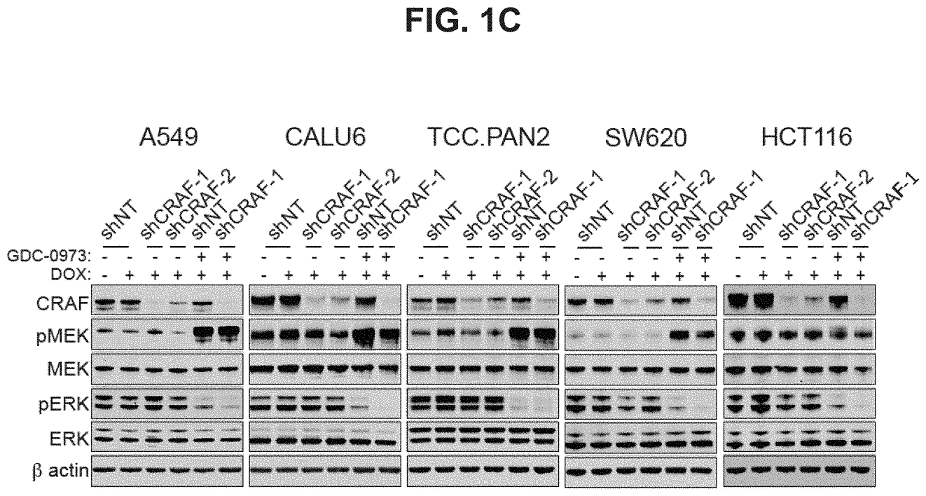

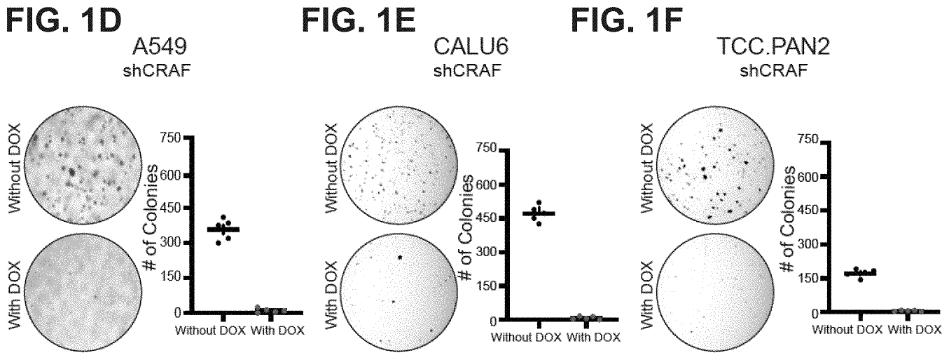

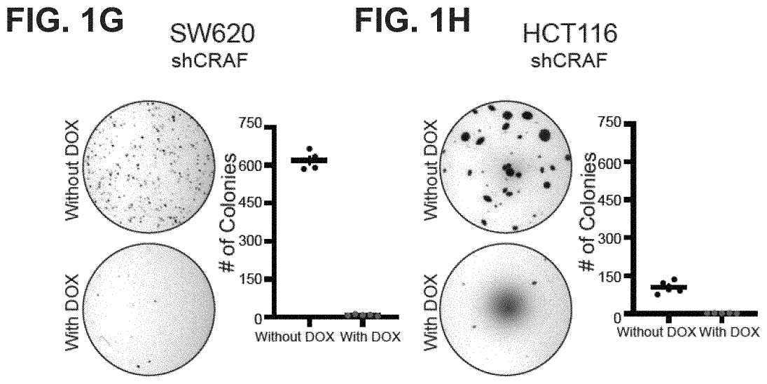

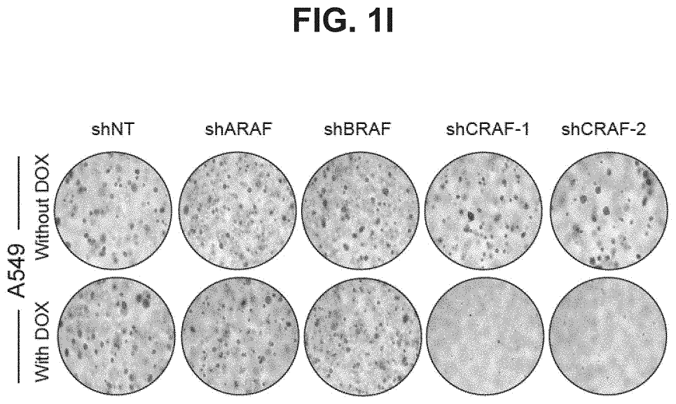

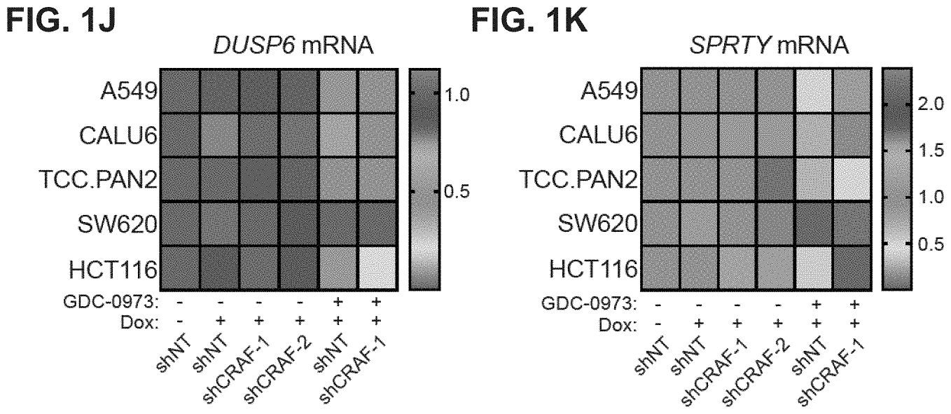

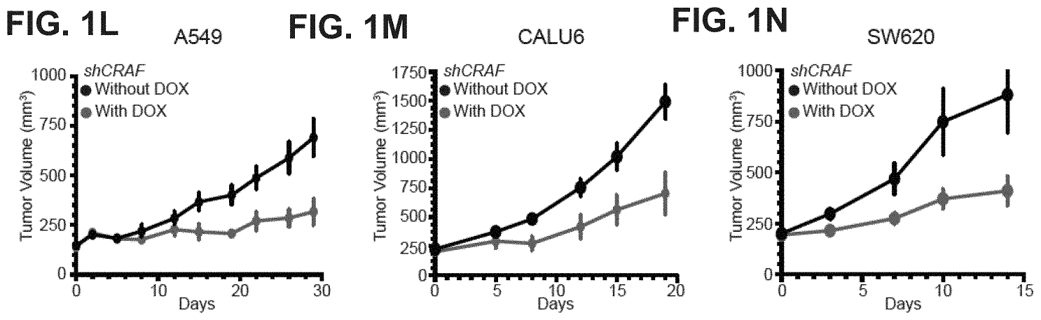

Analysis of CERES scores across subsets of RASK (KRAS), RASN (NRAS) and BRAF^{V600E} mutant tumor lines demonstrated that RASK (KRAS) and RASN (NRAS) mutant tumor lines were dependent on RAF1 (CRAF) for growth. Likewise, BRAF^{V600E} tumor lines were dependent on BRAF for growth (FIG. 1A). ARAF ablation across this panel of tumor cell lines had little effect, suggesting that ARAF has a distinct role from both BRAF and RAF1 (CRAF) (FIG. 1A). Deeper inspection of the cell lines demonstrated that a subset of RASK (KRAS) and RASN (NRAS) mutant cancer cell lines exhibited a strong dependency on RAF1 (CRAF) function for cell growth and survival (FIG. 1B).

b. RAF1 (CRAF) Depletion in RASK (KRAS) Mutant Cancer Cells

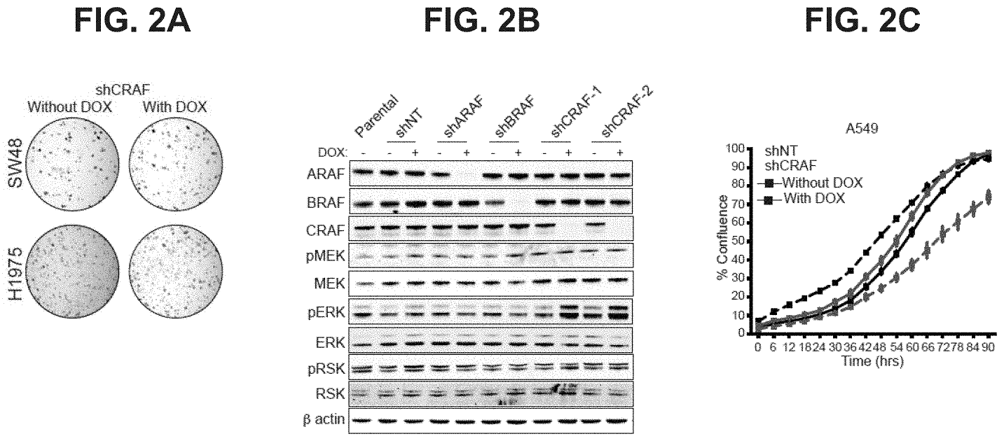

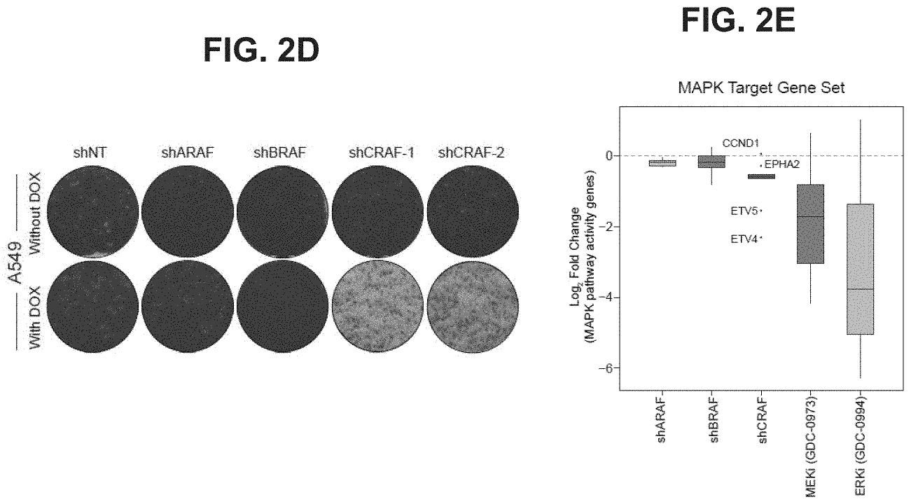

To confirm this observation, RAF1 (CRAF) was depleted in a panel of RASK (KRAS) mutant lung (A549, CALU6), pancreas (TCC.PAN2), and colon (SW620, HCT116) cancer cells. Unlike MEK inhibitor (GDC-0973) treatment, which suppresses MAPK signaling in RASK (KRAS) mutant cells, RAF1 (CRAF) depletion did not inhibit MAPK signaling (FIG. 1C). However, RAF1 (CRAF) depletion did significantly reduce the number of colonies formed on soft agar across the RASK (KRAS) mutant cell lines examined (FIGS. 1D-1H). Consistent with the notion that RAF1 (CRAF) dependency is observed in subsets of RASK (KRAS) mutant cells, RAF1 (CRAF) depletion in RAS wild-type human cancer cell lines (SW48, H₁₉₇₅) did not inhibit colony growth (FIG. 2A). As expected, depletion of ARAF or BRAF did not affect colony formation in the RASK (KRAS) mutant cells (FIGS. 1I and 2B). Interestingly, RAF1 (CRAF) depletion also had a modest effect on cell growth under 2D growth conditions, in contrast to the far stronger effect observed under anchorage-independent growth conditions (FIGS. 2C and 2D). This observation is similar to what was previously observed for RASK (KRAS) dependency in RASK (KRAS) mutant cell lines.

c. Gene Expression Analysis for MAPK Downstream Effectors

To confirm sustained MAPK activity in spite of RAF1 (CRAF) depletion, we performed a gene expression analysis for the MAPK downstream effectors DUSP6 and SPRTY upon RAF1 (CRAF) depletion in the examined RASK (KRAS) mutant cells lines. Again, RAF1 (CRAF) depletion did not affect DUSP6 (FIG. 1J) and SPRTY (FIG. 1K) mRNA expression in comparison to cells treated with a MEK inhibitor (GDC-0973). In order to delineate pathway alterations upon RAF1 (CRAF) depletion versus MAPK inhibition, RNA-sequencing analysis was performed. Treatment of RASK (KRAS) mutant cells with either a MEK inhibitor (GDC-0973) or an ERK inhibitor (GDC-0994) induced suppression of ERK downstream substrates (FIG. 2E). Conversely, acute depletion of either ARAF, BRAF, or RAF1 (CRAF) in these RASK (KRAS) mutant cells does not mediate inhibition of downstream MAPK targets. Thus, RAF kinases compensate to sustain MAPK signaling in comparison to MAPK inhibition.

d. RAF1 (CRAF) Depletion in RASK (KRAS) Mutant Xenograft Models

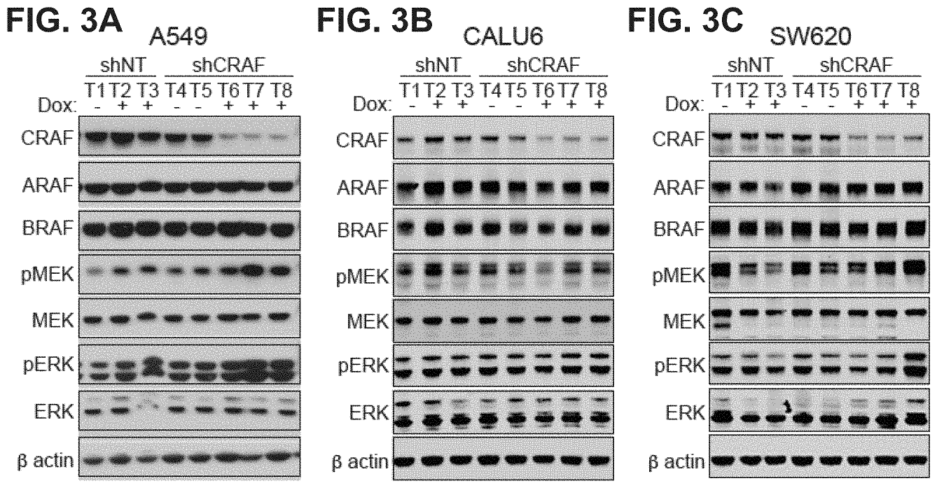

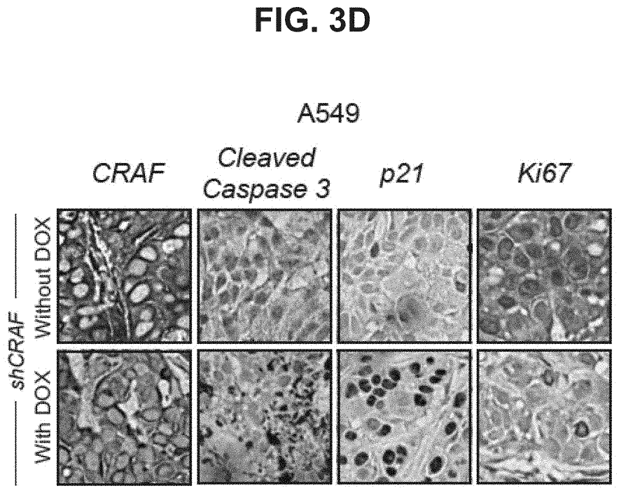

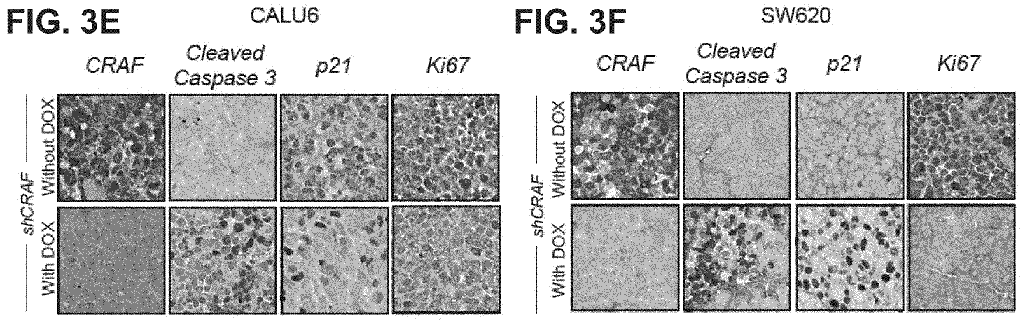

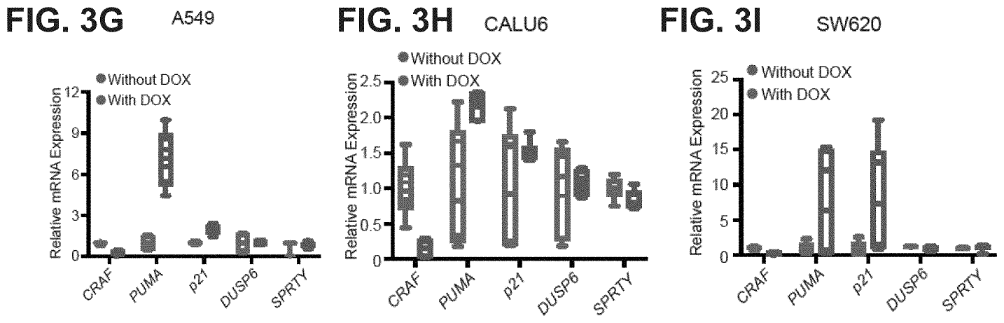

To further delineate RAF1 (CRAF) dependency in RASK (KRAS) mutant tumors in vivo, RAF1 (CRAF) depletion was monitored in three RASK (KRAS) mutant xenograft models (A549, CALU6 and SW620). RAF1 (CRAF) depletion resulted in significant tumor growth inhibition in all three xenograft models (FIGS. 1L-1N). Importantly, RAF1 (CRAF) ablation in established RASK (KRAS) mutant xenograft tumors did not inhibit MAPK signaling as reported sustained pERK expression (FIGS. 3A-3C). However, RAF1 (CRAF) loss did correlate with increased immunohistochemistry (IHC) staining (i.e., elevated expression) of cleaved caspase 3 and p21, indicating induction of cellular apoptosis and cell cycle arrest. Conversely, decreased IHC signal was observed for Ki67, a marker for cellular proliferation (FIGS. 3D-3F). Also, the apoptosis target gene PUMA and the cell cycle target gene p21 were upregulated in multiple RASK (KRAS) mutant tumors upon RAF1 (CRAF) depletion, while expression levels of DUSP6 and SPRTY remained unchanged (FIGS. 3D-31). Taken together, these data indicate that RAF1 (CRAF) loss is dispensable for ERK activation, but is required for growth of RASK (KRAS) mutant tumors.

### Example 2. Investigation of RAF Isoform Knockout Mutants in RASK (KRAS) Mutant Background

a. Investigation of RAF Isoform Knockout Mutants in RASK (KRAS) Mutant Background

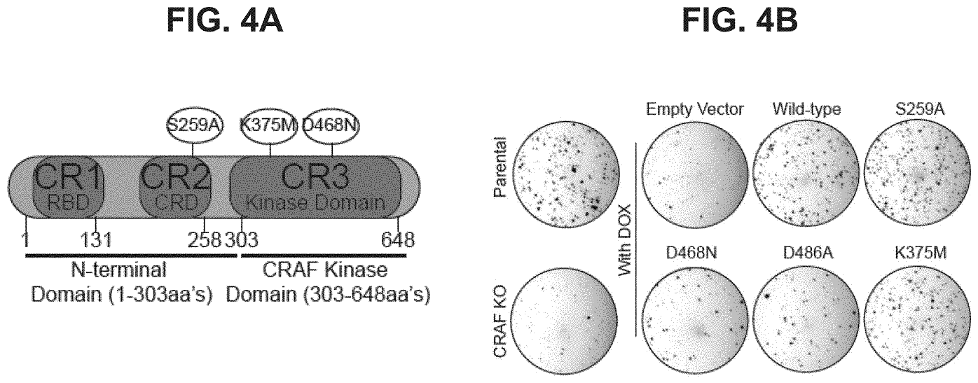

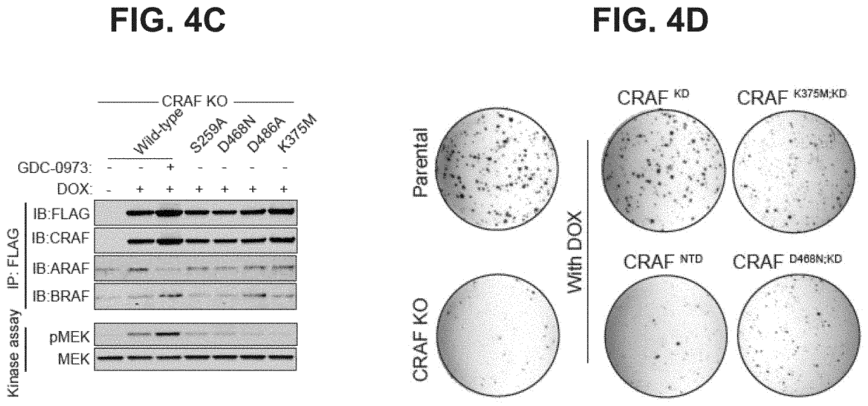

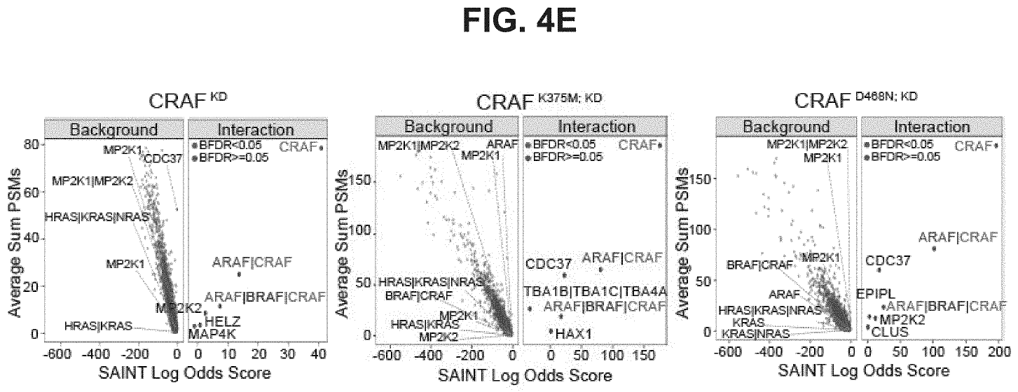

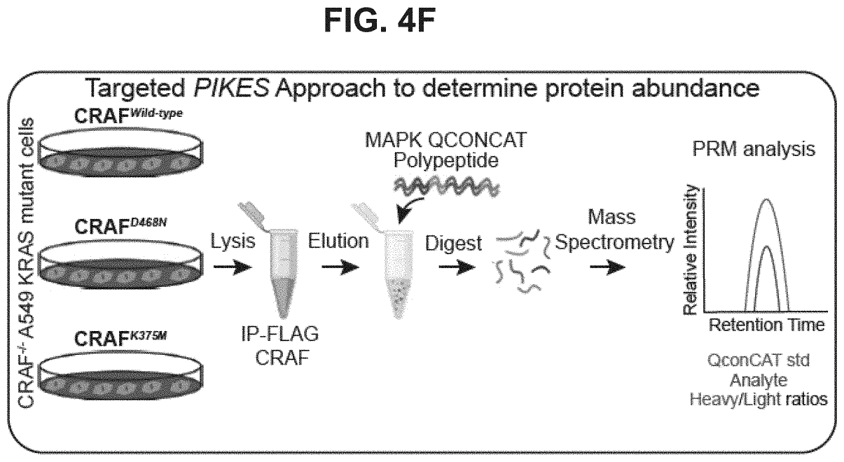

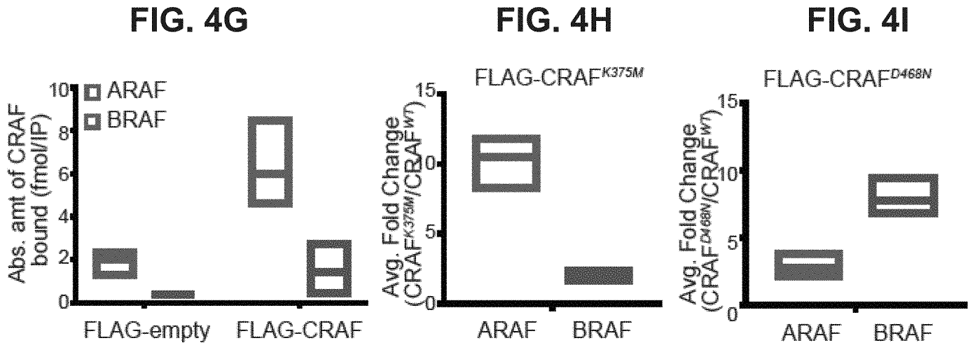

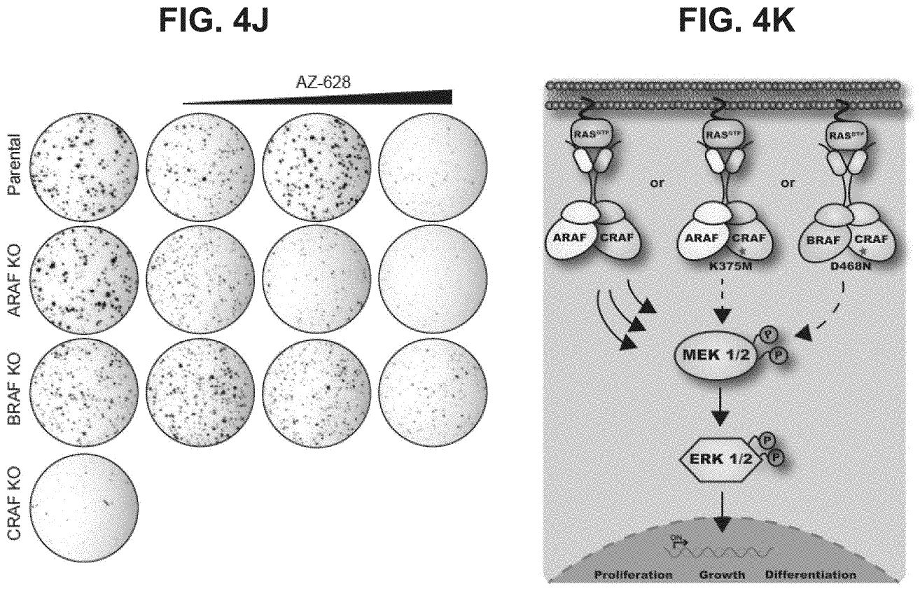

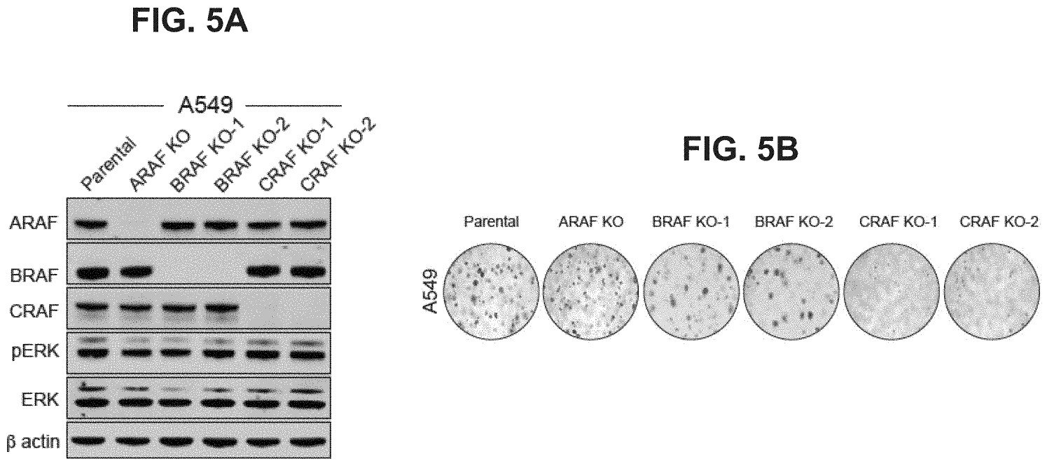

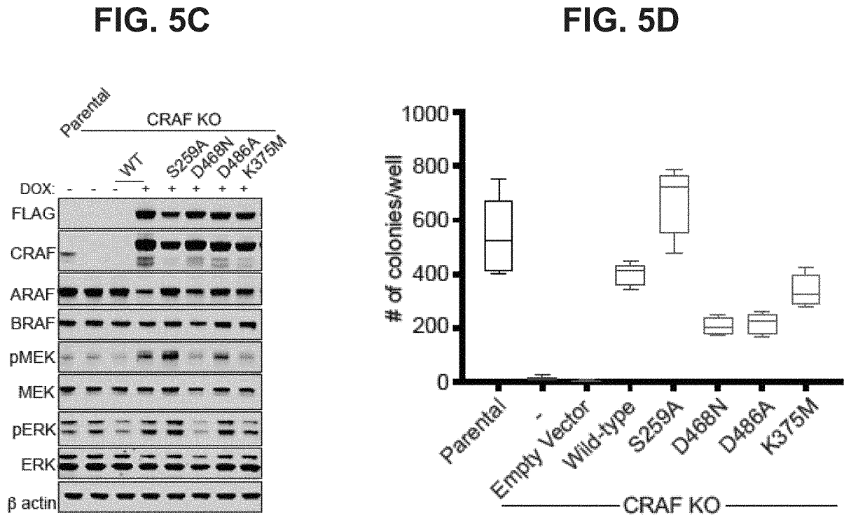

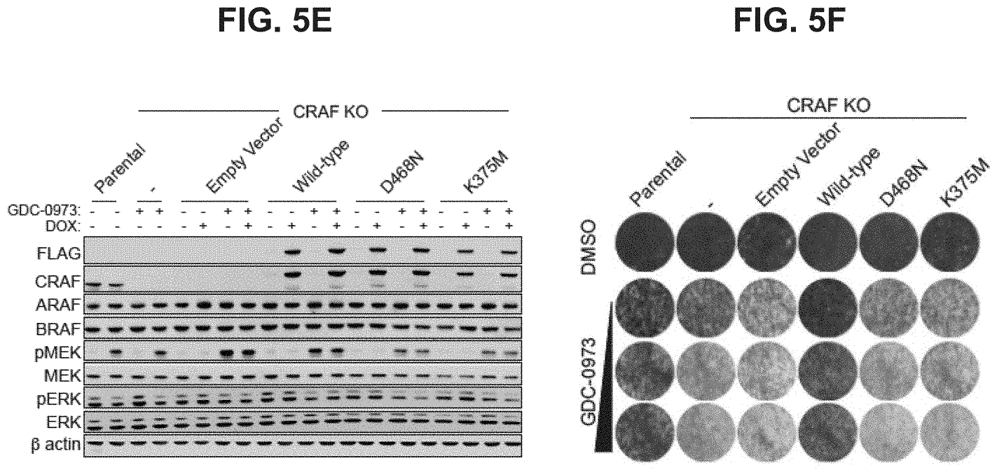

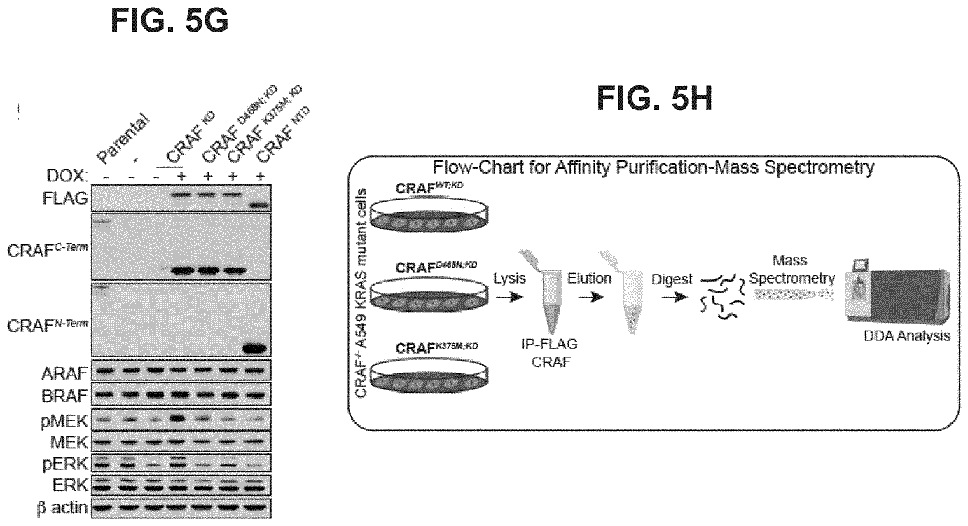

To test whether RAF1 (CRAF) kinase activity is required for RASK (KRAS) mutant tumor cell growth, knockout cell lines of the three RAF isoforms were generated in a RASK (KRAS) mutant background. RAF1 (CRAF) knockout clones demonstrated a significant reduction in colony formation compared to the parental cells, ARAF knockout cells, and BRAF knockout cells (FIGS. 5A and 5B). In the RAF1 (CRAF) knockout RASK (KRAS) mutant cells, stable expression of a panel of RAF1 (CRAF) mutants was established. The FLAG-tagged RAF1 (CRAF) expression constructs were titrated to express comparable levels of full-length RAF1 (CRAF) or the indicated mutants (FIGS. 4A and 5C). Expression of RAF1 (CRAF) kinase-dead mutants including RAF1^{D468N}, RAF1^{D486A}, and RAF1^{K375M}(CRAF^{D468N}, CRAF^{D486A}, and CRAF^{K375M}) reduced pERK levels compared to cells expressing RAF1^{wild-type} (CRAF^{wild-type}) or RAF1^{S259A} (CRAF^{S259A}), a mutation which renders the RAF1 (CRAF) kinase constitutively active (FIG. 5C). RAF1 (CRAF) kinase-dead mutants either partially (RAF1^{D468N} (CRAF^{D468N}), RAF1^{D486A}(CRAF^{D486A})) or fully (RAF1^{K375M} (CRAF^{K375M})) rescued colony growth in RAF1 (CRAF)-deficient RASK (KRAS) mutant cells (FIGS. 4B and 5D). In order to confirm that the RAF1 (CRAF) kinase dead mutants exhibited reduced kinase activity, RAF1 (CRAF) was immunoprecipitated and an in vitro kinase assay was conducted. Immunoprecipitation of FLAG-CRAF^{wild-type} and kinase-dead-CRAF^{D468N}, -CRAF^{D486A}, -CRAF^{K375M} mutants demonstrated heterodimerization with ARAF. This result was in contrast to the results of treatment with MEK inhibitor (GDC-0973), which enriched for RAF1:BRAF heterodimers (Hatzivassiliou et al., Nature, 464: 431-435, 2010; Poulikakos et al., Nature, 464: 427-430, 2010) (FIG. 4C). Functionally, the CRAF^{K375M} mutation completely abrogated MEK phosphorylation compared to CRAF^{wild-type}, but still rescued colony growth in RAF1-deficient RASK (KRAS) mutant cells (FIGS. 4C and 5C). It was previously shown that treatment of RASK (KRAS) mutant cell lines with MEK inhibitor induced BRAF:CRAF heterodimers and activated RAF1 (CRAF) kinase activity (Hatzivassiliou et al., Nature, 464: 431-435, 2010). Consistent with this, RAF1 (CRAF) kinase-dead mutants behaved like the RAF1 (CRAF) knockout, and were unable to rescue cell growth inhibition in the presence of MEK inhibitor compared to the cells expressing CRAF^{wild-type} (FIGS. 5E and 5F). Thus, whereas RASK (KRAS) mutant tumors are not dependent on RAF1 (CRAF) catalytic function for growth, treatment with MEK inhibitors confers dependence on RAF1 (CRAF) kinase activity through induction of BRAF and RAF1 (CRAF) heterodimers.

b. Determination of RAF1 (CRAF) Regions Required for RASK (KRAS)-Driven Tumorigenesis

In order to determine the regions of RAF1 (CRAF) required for RASK (KRAS)-driven tumorigenesis, constructs expressing the RAF1 (CRAF) N-terminal domain (CRAF^{NTD}, amino acids (aa) 1-303; SEQ ID NO: 99); the kinase domain only (CRAF^{KD}, aa 303-648; SEQ ID NO: 100); or the kinase domain with kinase-dead mutations (CRAF^{D468N;KD}, CRAF^{K375M;KD}; SEQ ID NO: 101 and SEQ ID NO: 102) were generated (FIG. 4A). These FLAG-RAF1 (FLAG-CRAF) kinase domain constructs were stably expressed in the RAF1 (CRAF) knockout RASK (KRAS) mutant cells. Active ERK signaling was observed upon expression of CRAF^{KD} and was dampened upon expression of CRAF^{D468N;KD} and CRAF^{K375M;KD} (FIG. 5G). Expression of RAF1 (CRAF) kinase-domain alone rescued colony growth in the RAF1 (CRAF)-ablated RASK (KRAS) mutant cells (FIG. 4D). Similar to the CRAF^{KD}, the kinase dead CRAF^{D468N;KD} and CRAF^{K375M;KD} rescued cell growth when expressed in RAF1 (CRAF)-deficient RASK (KRAS) mutant cells (FIG. 4D). Conversely, expression of CRAF^{NTD} did not rescue cell growth (FIG. 4D). Taken together, this data suggests that the kinase domain of RAF1 (CRAF), but not its kinase activity, is required for RASK (KRAS) driven tumor cell growth.

### Example 3. Identification of Interactors of CRAF^{KD}, CRAF^{D468N;KD}, and CRAF^{K375M;KD} Using PIKES

a. AP-MS Experiments

In general, RAF kinases homodimerize and heterodimerize, as well as interact with the substrate MEK, through their kinase domains. Unbiased affinity purification mass spectrometry (AP-MS) experiments were conducted to identify potential interactors of CRAF^{KD}, CRAF^{D468N;KD} and CRAF^{K375M;KD} (FIG. 5H). Surprisingly, AP-MS experiments revealed that RAF1 (CRAF) protein interactors were limited to members of the MAPK pathway (FIG. 4E). FIG. 4E provides Statistical Analysis of Interactomes (SAINT) log odds scores: SAINT is an algorithm that reports a log odds score describing the likelihood that proteins identified in a protein-protein experiment (e.g., an AP-MS experiment) are specifically enriched by the bait protein of interest (e.g., RAF1 (CRAF)), rather than enriched by chance. The sum of peptide-spectrum match values (PSMs) for each protein group from bait and control searches were used as input for the Statistical Analysis of Interactome (SAINT) algorithm (v3.6.1) to determine prey-bait interactions. Positive log odds scores indicate the likelihood that the interaction is true rather than false (null model).

Both active and kinase-dead FLAG-tagged CRAF^{KD} interacted with ARAF and BRAF. Whereas the ARAF peptides initially appeared to be more enriched than BRAF, the ARAF peptides could not be unambiguously distinguished from the other RAF isoforms in this assay due to significant homology of protein sequences in the kinase domain of the three RAF isoforms.

b. Use of PIKES to Distinguish Between RAF Isoforms

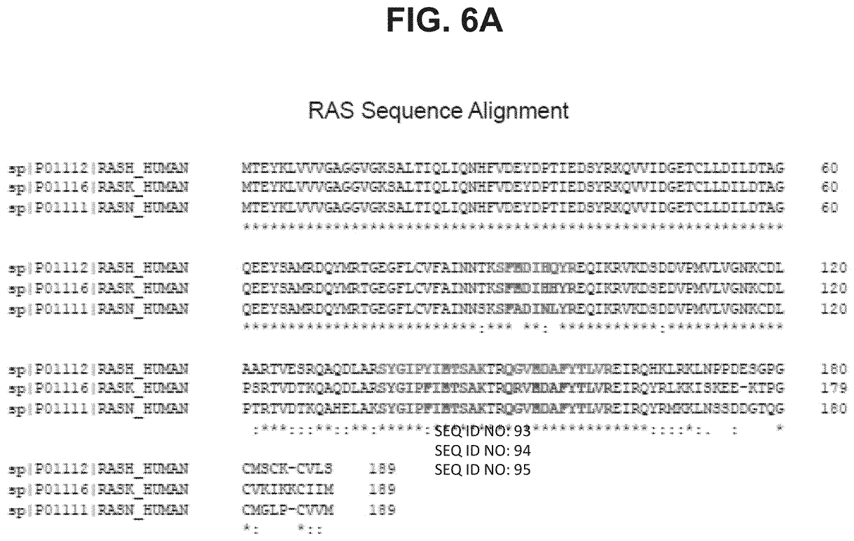

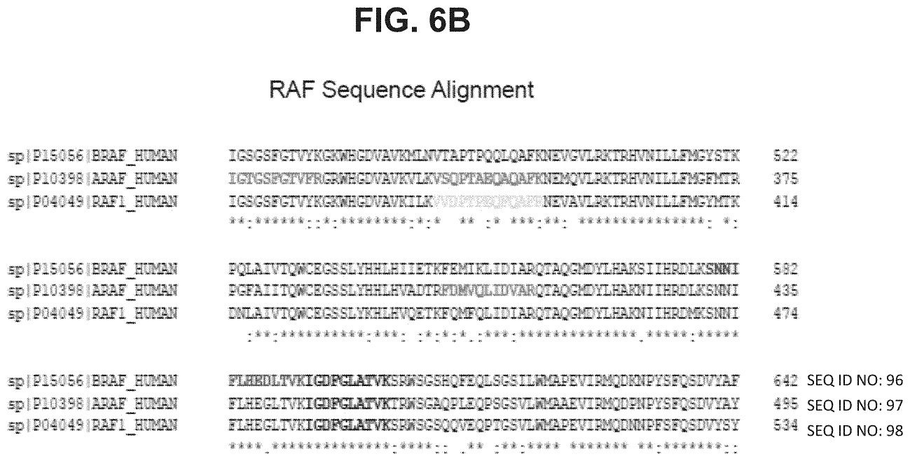

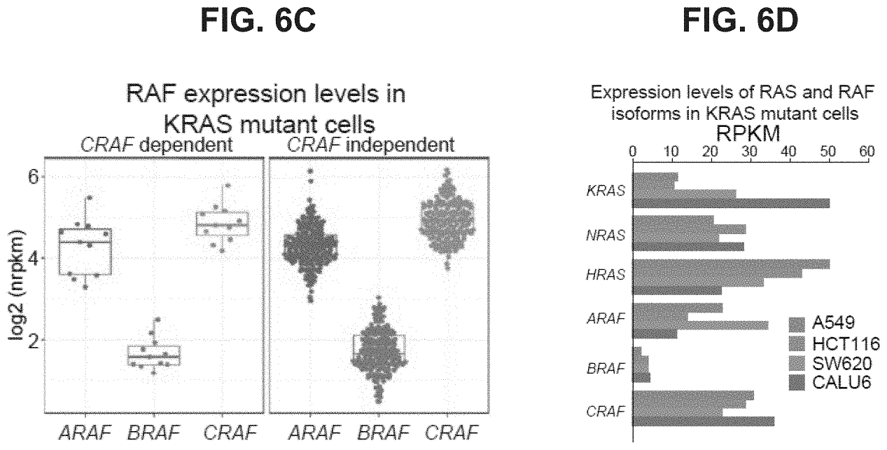

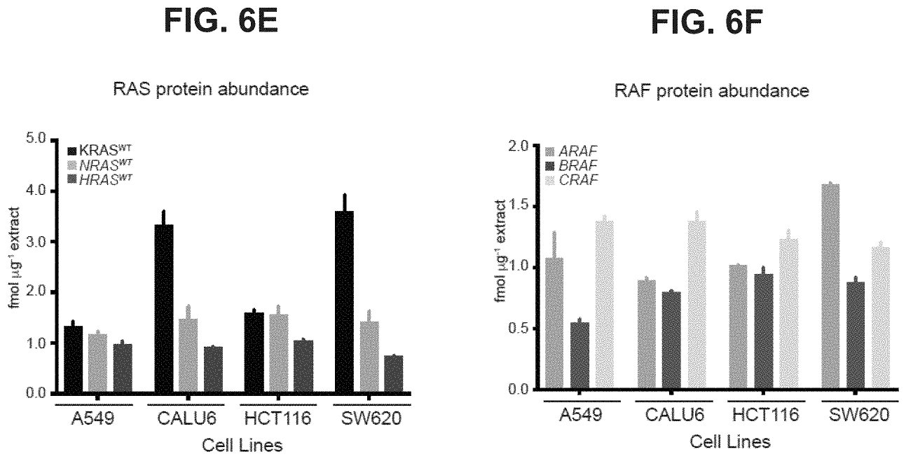

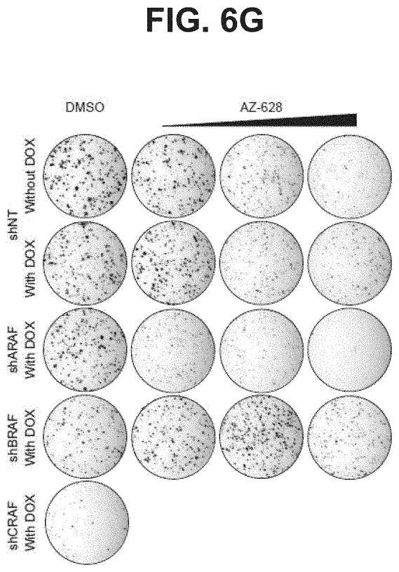

In order to distinguish between the various RAF isoforms and quantify the protein abundance of the RAF dimerization partners, a recently described quantitative mass spectrometry approach termed Protein Interaction Kinetics and Estimation of Stoichiometry (PIKES) (Reichermeier et al., Mol Cell, 77: 1092-1106 el 099, 2020) was utilized. At the heart of the PIKES approach is an isotopically labeled internal standard artificial protein assembled by concatenating a series of peptides from proteins of interest. An isotopically labeled internal standard protein was assembled by concatenating a series of peptides (both isoform-specific and pan-peptides) from the RAS/MAPK pathway including the three RAS and RAF isoforms. Upon proteolytic digestion, this QconCAT standard (Pratt et al., Nat Protoc, 1: 1029-1043, 2006) is converted into equimolar ratios of isotopically labelled internal standard peptides that can be used to distinguish between and estimate the stoichiometries of closely related pathway components, such as the RAS and RAF isoforms (FIGS. 6A and 6B) or the corresponding wild-type and mutant versions.

Parallel reaction monitoring-based mass spectrometry (PRM-MS) was used to measure the signal intensity of each MAPK pathway component in RASK (KRAS) mutant cells relative to a corresponding internal standard polypeptide (derived from the QconCAT) labeled with either heavy arginine (R10) or lysine (K8) (FIG. 4F). To test the functionality of the assay, the abundances of the MAPK pathway components across a panel of RASK (KRAS) mutant cell lines were first determined using mRNA expression levels of RAS and RAF isoforms in the RASK (KRAS) mutant cell lines from the Project Achilles dataset as reference. Significantly higher mRNA expression levels (RKPM) of ARAF and RAF1 (CRAF) were observed relative to BRAF in the RASK (KRAS) mutant cells (FIGS. 6C and 6D). Next, the protein abundances of the RAS family members RASH (HRAS), RASN (NRAS) and RASK (KRAS), as well as the RAF kinases ARAF, BRAF and RAF1 (CRAF), were determined across a panel of RASK (KRAS) tumor cell lines by utilizing the distinguishing and RAS mutant-specific peptides of the QconCAT (SEQ ID NO: 1) (FIGS. 6E and 6F). Protein expression levels of RAS isoforms differed based upon the ratio of wild-type to mutant RASK (KRAS) expressed. Correlatively, CALU6 and SW620 cell lines have the highest levels of RASK (KRAS) protein (FIG. 6E). Consistent with the mRNA expression data, higher levels of ARAF and RAF1 (CRAF) compared to BRAF protein were observed in all of the four RASK (KRAS) mutant cell lines tested (FIG. 6F).

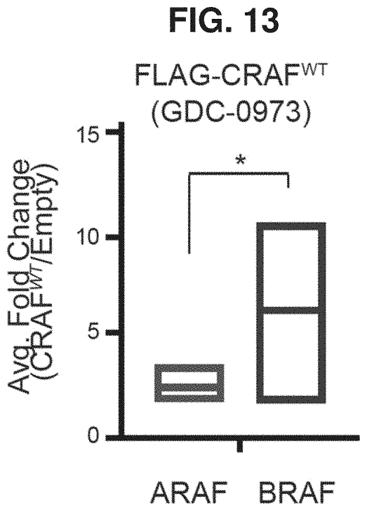

The PIKES approach was next used to determine whether RAF1 (CRAF) preferentially interacted with either BRAF or ARAF in RASK (KRAS) mutant cells (FIG. 4F). FLAG-tagged CRAF^{wild-type} and the kinase-dead mutants CRAF^{D468N} and CRAF^{K375M} were expressed in and immunoprecipitated from RASK (KRAS) mutant cells lacking endogenous RAF1 (CRAF). Higher levels of RAF1 (CRAF):ARAF dimers than RAF1 (CRAF):BRAF dimers were consistently observed in RASK (KRAS) mutant cells expressing CRAF^{wild-type} and CRAF^{K375M} (FIG. 4G). To further validate the preferential RAF dimer association, CRAF^{wild-type} was immunoprecipitated upon treatment with a MEK inhibitor (GDC-0973). As previously demonstrated, treatment with the MEK inhibitor promoted increased CRAF: BRAF dimers in comparison to CRAF: ARAF dimers (FIG. 13). To determine whether a preferential dimer association exists in the context of CRAF kinase-dead mutants, the CRAF^{K375M} kinase-dead mutant was immunoprecipitated and increased ARAF: CRAF dimers were observed (FIG. 4H). In contrast, the other kinase-dead mutant CRAF^{D468N} favored for RAF1 (CRAF):BRAF dimers (FIG. 4I), which was consistent with previous reports demonstrating that CRAF-associated Noonan's syndrome mutations in the HRD (D468) and DFG (D486) motif of CRAF dimerize with BRAF to activate ERK signaling (Wu et al., Mol Cell Biol, 32: 3872-3890, 2012). Thus, in RASK (KRAS) mutant cells, RAF1 (CRAF) preferentially binds to ARAF, potentially due to the increased abundance of ARAF versus BRAF observed across all KRAS mutant tumor lines (FIGS. 4H and 6F).

c. PIKES Analysis Methods

Parallel reaction monitoring (PRM) analysis was used to quantify the stoichiometric relationships between various wild-type and mutant MAPK components using a custom-designed QconCAT reagent (synthesis of QconCAT reagent by PolyQuant GmbH). The MAPK QconCAT comprised 42 peptides from 10 proteins concatenated into a single isotopically labeled polypeptide. Stable isotopes were incorporated at lysine (K8; 13C615N2) and arginine (R10; 13C615N4) residues. For key disease-associated mutations, wild-type and mutant sequences were incorporated into the MAPK QCONCAT to facilitate distinct quantitative assays reporting the abundance of each form. Peptides used in the PIKES analysis for distinguishing RAS and RAF isoforms, including mutant sequences, are shown in Table 6. The pan-RAS peptide (LVVVGAGGVGK (SEQ ID NO: 35)) sequence is shared across all RAS isoforms and includes the G12 and G13 codon where the most common disease-associated mutations of RAS are found. RAS mutant-specific peptides are derived from the pan-RAS peptide and detect for G12/13V, G12/13D, and G12/13S mutations.

TABLE 6
Isoform-specific and pan-QCONCAT
peptide sequences used for PIKES
analysis for RAF and RAS isoforms

| Protein | Peptide | Genotype |
|---|---|---|
| RAF1 | VVDPTPEQFQAFR (SEQ ID NO: 2) | Wild-type |
| BRAF | SNNIFLHEDLTVK (SEQ ID NO: 5) | Wild-type |
| ARAF | IGTGSFGTVFR (SEQ ID NO: 12) | Wild-type |
| RAF1\| | IGDFGLATVK (SEQ ID NO: 14) | Wild-type |
| BRAF\| | | |
| ARAF | | |
| KRAS | SFEDIHHYR (SEQ ID NO: 26) | Wild-type |
| NRAS | SFADINLYR (SEQ ID NO: 28) | Wild-type |
| HRAS | SFEDIHQYR (SEQ ID NO: 30) | Wild-type |
| KRAS\| | LVVVGAGGVGK (SEQ ID NO: 35) | Wild-type |
| NRAS\| | | |
| HRAS | | |
| KRAS\| | LVVVGAVGVGK (SEQ ID NO: 39) | Mutant- |
| NRAS\| | | G12/13V |
| HRAS | | |
| KRAS\| | LVVVGADGVGK (SEQ ID NO: 42) | Mutant- |
| NRAS\| | | G12/13D |
| HRAS | | |
| KRAS\| | LVVVGASGVGK (SEQ ID NO: 43) | Mutant- |
| NRAS\| | | G12/13S |
| HRAS | | |

For PIKES experiments, a total of 2×10⁷ cells were lysed in cell lysis buffer (8M urea, 50 mM Tris-HCl pH8.0) supplemented with protease (Roche, #11836170001) and phosphatase inhibitors (Thermo, #78426). Total protein was quantified using the PIERCE™ BCA Protein Assay Kit (Thermo Fisher, 23227). One aliquot of 100 μg of cell lysate was combined with the MAPK QconCAT reagent (50 fmol/1 μg cell lysate) for each replicate sample. Cell lysates were reduced with 10 mM DTT for 15 minutes at 50° C., and allowed to cool to room temperature prior to alkylation with 30 mM iodoacetamide for 15 minutes in the dark. Lysates were diluted 4-fold to <2M urea and digested at 37° C. overnight with modified sequencing-grade trypsin (Promega) using 1:50 enzyme-to-protein ratio. Digested peptides were acidified to 0.1% trifluoroacetic acid (TFA) final concentration and desalted using 5 μL C18 cartridges on the AssayMAP Bravo platform (Agilent) running the Peptide Cleanup v2.0 method. Lyophilized peptides were resuspended in 100 μL Buffer A (0.1% formic acid/2% acetonitrile/98% water). LC-MS/MS was performed on an ORBITRAP FUSION™ LUMOS™ mass spectrometer (Thermo Fisher) coupled to a DIONEX™ ULTIMATE™ 3000 rapid separation liquid chromatography (RSLC) system. For each sample, 1 μL of digested peptides were injected on an lonOpticks Aurora Series column and separated over a 40 minute gradient of buffer B (0.1% formic acid (FA)/98% acetonitrile (ACN)/2% water) from 2% to 35% and a second stage gradient over 4.9 minutes from 35% to 75%. The ORBITRAP FUSION™ LUMOS™ was operated with precursor ions analyzed by the ORBITRAP FUSION™ LUMOS™ at 240,000 resolution, automatic gain control (AGC) target at 1×10⁶, and a maximum injection time of 50 ms. Trigger peptides were added into an inclusion list with corresponding m/z, charge state, and retention time window of 6 minutes. Selected trigger peptides for MS2 were fragmented by collision-induced dissociation (CID) with a collision energy of 30%, and analyzed in the ORBITRAP FUSION™ LUMOS™ at 15,000 resolution with an AGC target of 2×10⁵ and a maximum injection time of 120 ms. For RAF trigger peptides, a maximum injection time of 220 ms was used.

To quantify RAF heterodimers, FLAG-IP of CRAF was performed as stated above and eluates were obtained by incubating bound beads with 3×FLAG® Peptide (Sigma, #F4799) in 2M urea and 50 mM Tris-HCl pH8.0 for 30 minutes with shaking at room temperature. Collected eluates were then combined with 100 fmol of QconCAT reagent and samples were processed similarly to the cell line analysis above for PRM analysis.

For PRM analysis, RAW files were loaded onto Skyline (v19.1). A target peptide list was generated from the QconCAT peptides for both unlabeled and heavy-labeled peptides with transition setting filtered for precursor charges 2 and 3, ion charges 1 and 2, and y-ion types. Selected peaks from product ions were reviewed manually. Both MS1 and MS/MS filtering were set to Orbitrap mass analyzer with 60,000 resolving power. Signal was summed for the top three product ions and normalized to the heavy labeled QconCAT internal standard for peptide quantification.

### Example 4. Investigation of Ratios of RAF1 (CRAF):ARAF to RAF1 (CRAF):BRAF Heterodimers

It was hypothesized that the ratio of RAF1 (CRAF):ARAF versus RAF1 (CRAF):BRAF heterodimers may be important in the growth of RASK (KRAS) mutant tumors, and that this ratio may impact the efficacy of RAF kinase inhibition. To test this hypothesis, RASK (KRAS) mutant cells depleted of individual members of the RAF kinases (ARAF, BRAF or RAF1 (CRAF)) were treated with a pan-RAF dimer inhibitor (AZ-628). ARAF ablation in RASK (KRAS) mutant cells sensitized cells to RAF dimer inhibition (FIGS. 4J and 6G). This suggests that RAF1 (CRAF):BRAF heterodimers promote RAF1 (CRAF) kinase activity in RASK (KRAS) mutant cells, which is inhibited with a RAF dimer inhibitor. On the contrary, BRAF ablation in RASK (KRAS) mutant cells rendered the cell less sensitive to RAF dimer inhibition, suggesting that RAF1 (CRAF):ARAF dimers are more resistant to kinase inhibition. Taken together, RAF1 (CRAF) dimerization partners appear to dictate sensitivity to MAPK inhibition.

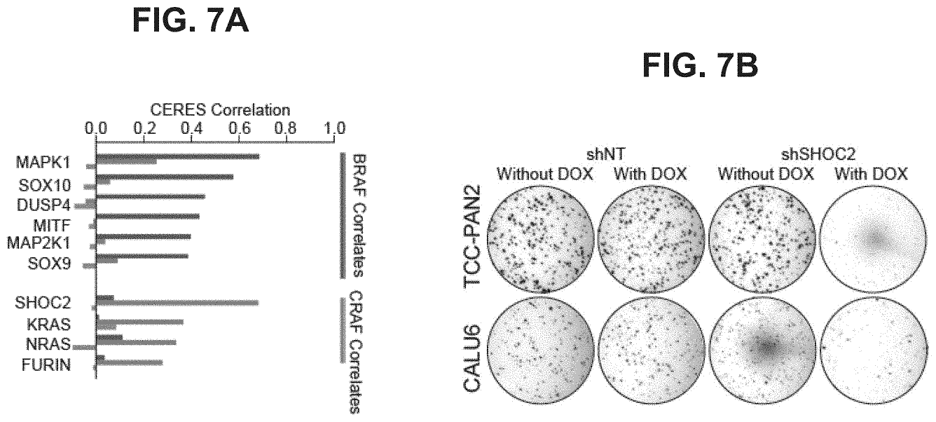

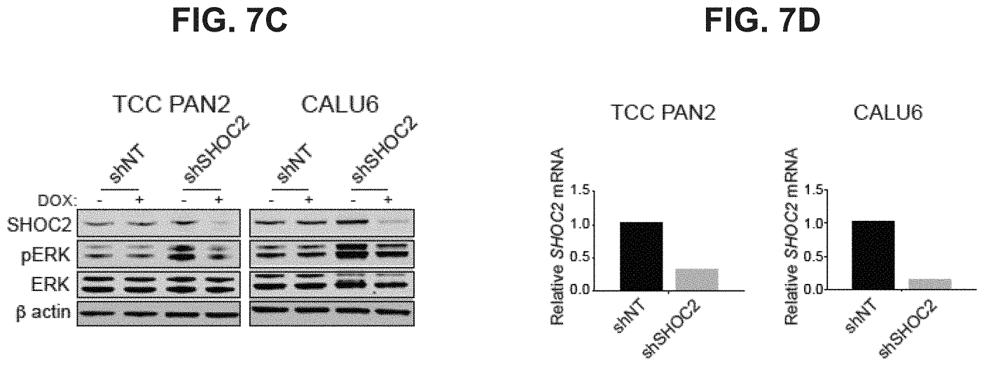

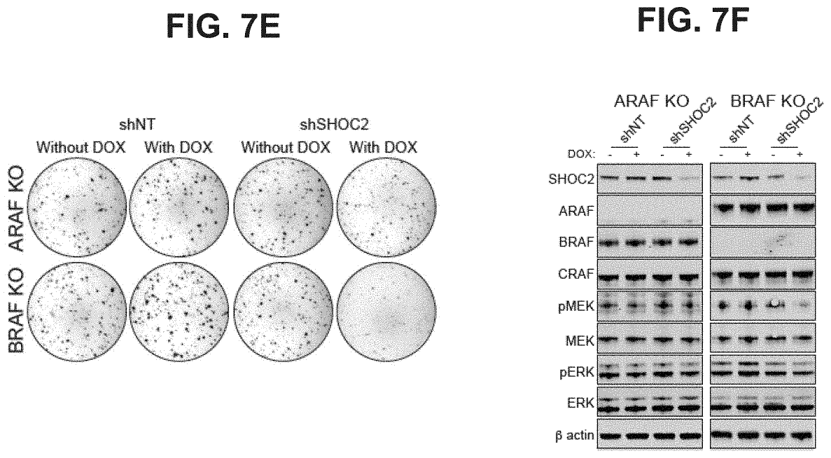

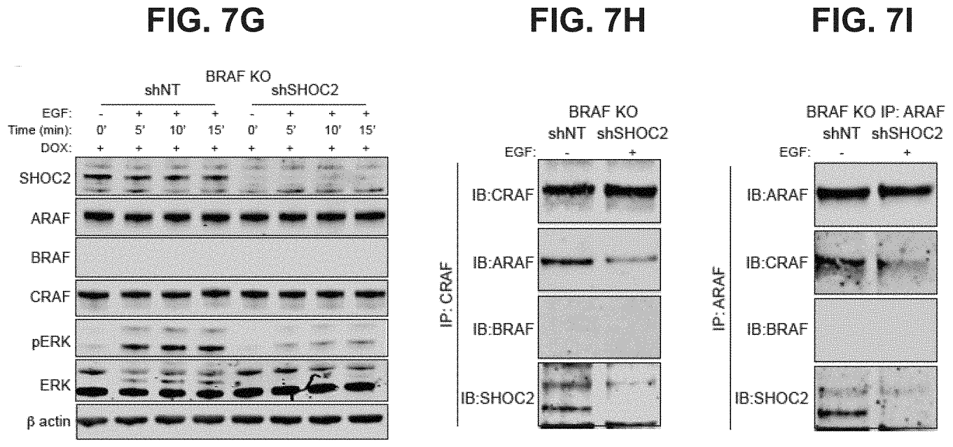

Given that RASK (KRAS) mutant cells have higher levels of RAF1 (CRAF):ARAF heterodimers than RAF1 (CRAF):BRAF heterodimers, it was necessary to characterize the functional roles of RAF1 (CRAF) heterodimers. A genetic approach targeting SHOC2, a member of the MRAS and PP1 complex which regulates RAF dimer formation, was used (Boned del Rio et al., Proc Natl Acad Sci USA, 116: 13330-13339, 2019; Jones et al., Nat Commun, 10: 2532, 2019). Importantly, SHOC2 was identified as the gene for which depletion was most highly correlated with RAF1 (CRAF) depletion in the Achilles DepMap portal, meaning that tumor cell lines that were dependent on RAF1 (CRAF) were likely to be also dependent on SHOC2 depletion (FIG. 7A). This observation was confirmed by depleting SHOC2 in RASK (KRAS) mutant cells (FIGS. 7B-7D). SHOC2 depletion has been demonstrated to prevent RAF dimer formation as a mechanism for inhibiting cell growth (Boned del Rio et al., Proc Natl Acad Sci USA, 116: 13330-13339, 2019; Jones et al., Nat Commun, 10: 2532, 2019). To determine the functional role of RAF1 (CRAF):ARAF and RAF1 (CRAF):BRAF heterodimers, SHOC2 was depleted in RAF knockout A549 RASK (KRAS) mutant cells. Previously, A549 cells were demonstrated to be insensitive to SHOC2 ablation (Jones et al., Nat Commun, 10: 2532, 2019). However, A549 BRAF knockout cells, which would favor RAF1 (CRAF):ARAF dimers, were sensitive to SHOC2 ablation, having reduced colony formation compared to A549 ARAF knockout cells that favor RAF1 (CRAF):BRAF dimers (FIGS. 7E and 7F). Mechanistically, SHOC2 ablation disrupts heterodimerization between RAF kinases, as demonstrated by the reduction of immunoprecipitated RAF1 (CRAF) or ARAF in RASK (KRAS) mutant cells upon SHOC2 depletion (FIGS. 7G-71). Thus, SHOC2 knockdown-mediated disruption of RAF1 (CRAF):ARAF heterodimers reduces cell growth in RASK (KRAS) mutant cells.

### Example 5. Functional Role of ARAF in RASK (KRAS) Mutant Cells

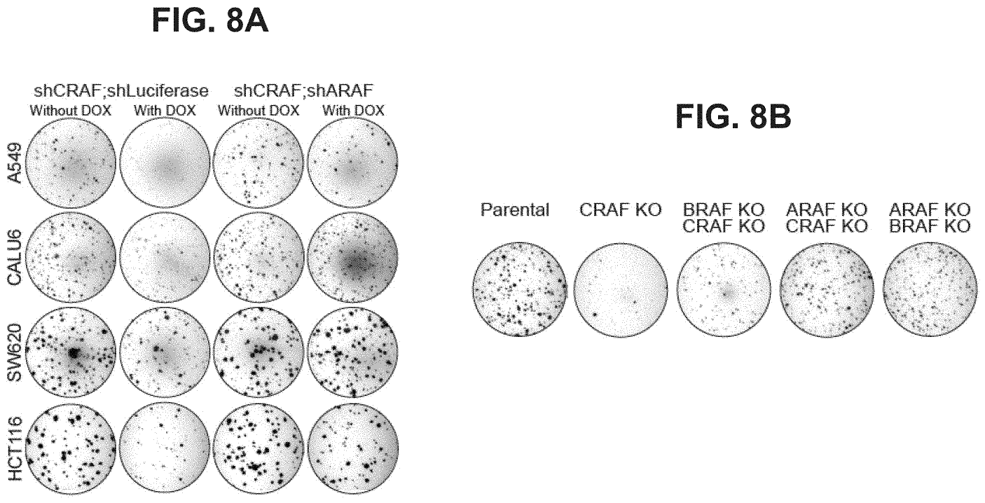

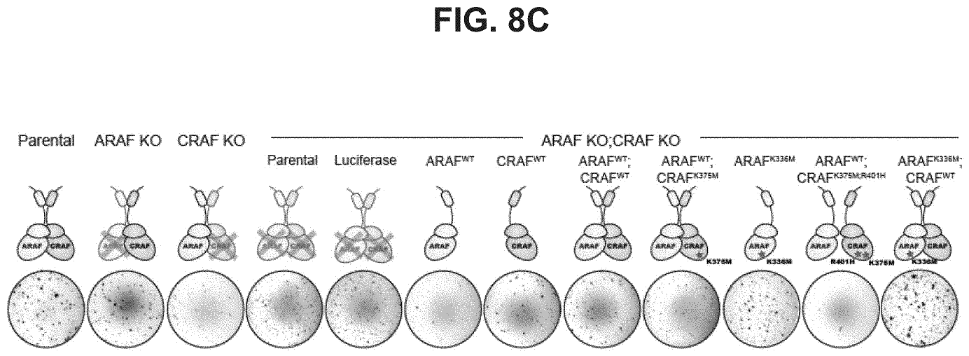

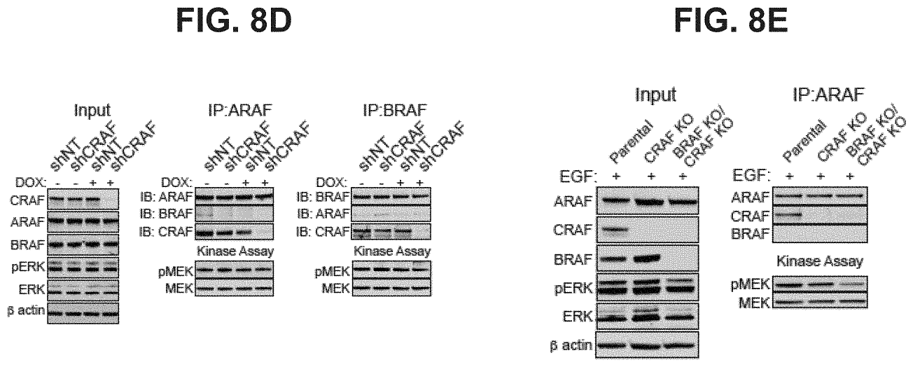

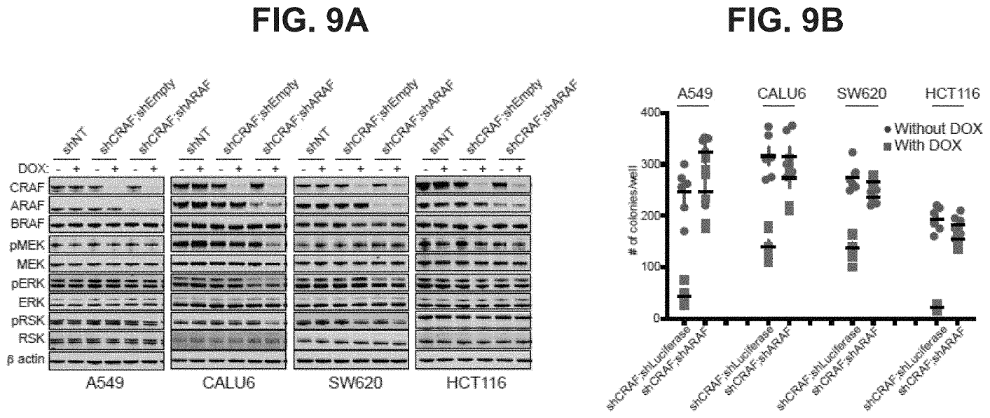

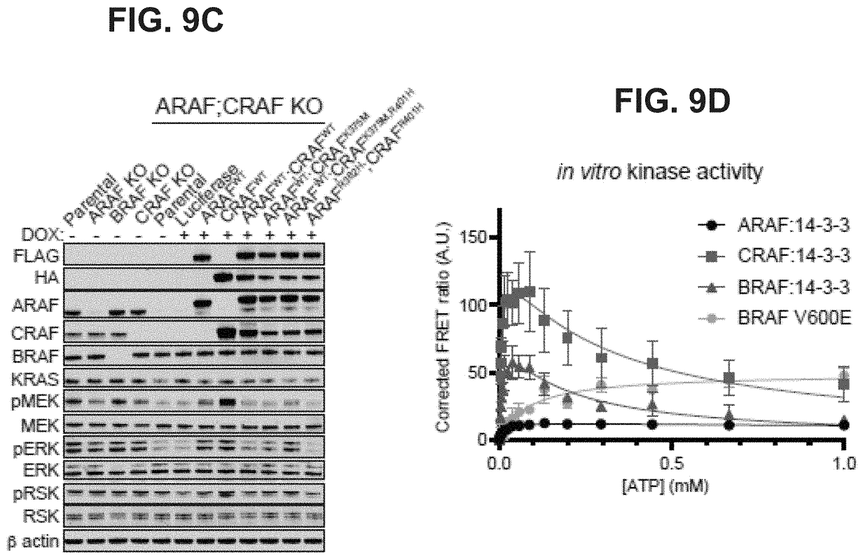

Given the significance of RAF1 (CRAF):ARAF heterodimers in promoting growth of RASK (KRAS) mutant cells, the functional role of ARAF was examined by depletion of both RAF1 (CRAF) and ARAF. Co-depletion of RAF1 (CRAF) and ARAF rescued colony growth in RAF1 (CRAF)-deficient cells (FIGS. 8A, 9A, and 9B). Next, double CRISPR double knockout clones of the RAF isoforms were generated in RASK (KRAS) mutant A549 cells. ARAF:RAF1 (CRAF) double knockout cells rescued the cell death mediated by RAF1 (CRAF) loss, while BRAF:RAF1 (CRAF) double knockout cells had reduced colony formation (FIG. 8B). The ARAF and BRAF double knockout cells resembled the parental cells (FIG. 3B). This indicates that ARAF homodimers function in limiting colony growth in RASK (KRAS) mutant cancer cells. To test the functional role of RAF1 (CRAF):ARAF heterodimers, FLAG-tagged ARAF and HA-tagged RAF1 (CRAF) constructs were expressed stably in ARAF and RAF1 (CRAF) double knockout cells (FIG. 9C). Expression of ARAF^{wild-type} inhibited colony formation in the ARAF;RAF1 (CRAF) double knockout cells, suggesting that ARAF homodimers limit growth of RASK (KRAS) mutant tumors (FIG. 8C). However, expression of CRAF^{wild-type} or co-expression of ARAF^{wild-type};CRAF^{wild-type} or the kinase-dead mutants ARAF^{wild-type};CRAF^{K375M} promotes growth of RASK (KRAS) mutant cells (FIG. 8C). Conversely, disruption of RAF1 (CRAF):ARAF heterodimers ARAF^{wild-type};CRAF^{K375M};R^{401H} mediates cell growth inhibition in the ARAF;RAF1 (CRAF) double knockout cells (FIG. 8C). Importantly, expression of ARAF^{wild-type}, and not kinase-dead ARAF^{K336M}, prevented growth of ARAF;RAF1 (CRAF) double knockout cells (FIG. 8C), indicating that the catalytic activity of ARAF homodimers mediates cell growth inhibition upon RAF1 (CRAF) loss in RASK (KRAS) mutant cells.

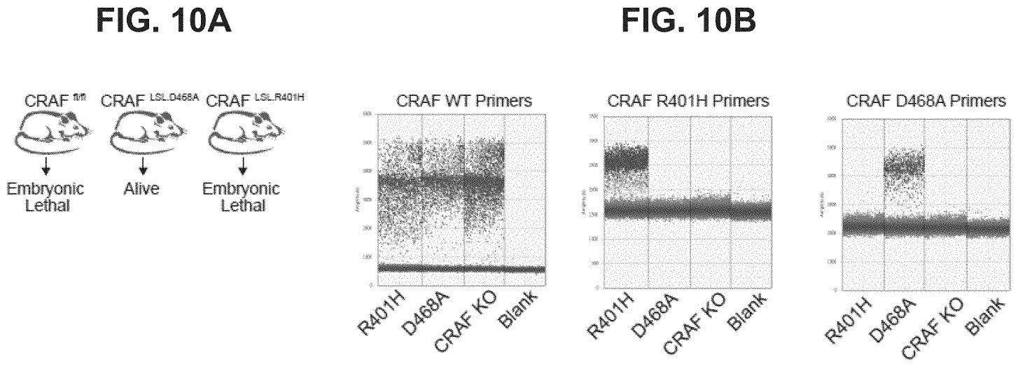

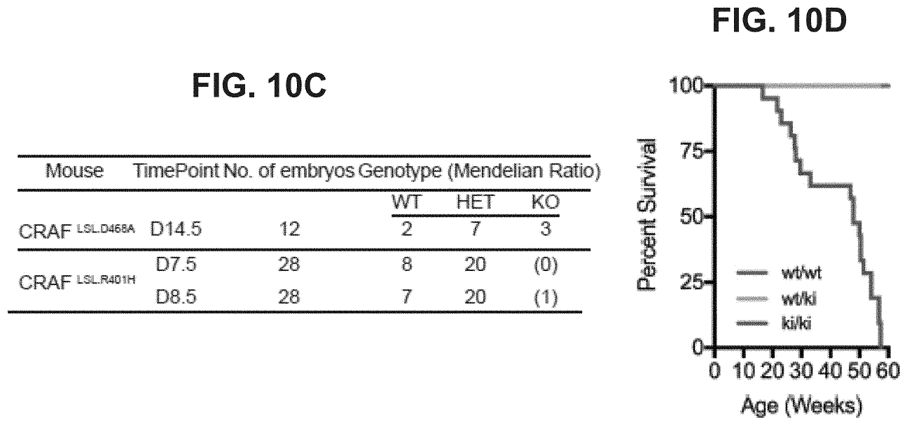

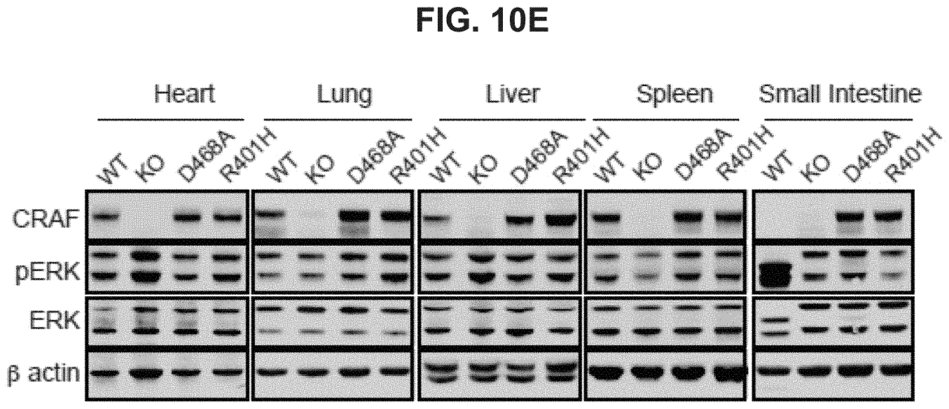

To more precisely differentiate the role of RAF1 (CRAF) dimerization from RAF1 (CRAF) catalytic activity, kinase-dead CRAF^{D468A} and dimer-defective CRAF^{R401H} knock-in mice were generated, along with RAF1 (CRAF) conditional knockout (CRAF^{fl/fl}) mice (FIGS. 10A and 10B). As expected, CRAF^{D468A} mice rescued embryonic lethality of CRAF^{fl/fl}, with a median survival of 50 weeks (FIGS. 10C and 10D). Surprisingly, the CRAF^{R401H} knock-in mice, which cannot form RAF1 (CRAF) dimers, were embryonic lethal as early as E6.5, similar to the CRAF^{fl/fl} knockout mice (FIG. 10C). To confirm expression of RAF1 (CRAF) kinase dead and dimer defective mutants, a homozygous CRAF^{fl/fl}, CRAF^{LSL.D468A} and CRAF^{LSL.R401H} mouse was crossed to a ubiquitous Rosa²⁶.Cre.ER^{T2} reporter line. Stable expression of RAF1 (CRAF) was observed in all tissues up to 30 days post tamoxifen (FIG. 10E). Thus, RAF1 (CRAF) kinase activity and dimerization appear to be distinguished from one another during development, with RAF1 (CRAF) catalytic function being dispensable and RAF1 (CRAF) dimerization required for survival.

Among the RAF family of kinases, ARAF is less understood due to its minimal kinase activity (Marais et al., J Biol Chem, 272: 4378-4383). It was hypothesized that ARAF function is negatively regulated by RAF1 (CRAF) in RASK (KRAS) mutant cells. To determine whether the catalytic function of ARAF is impacted by the absence of RAF1 (CRAF), a MEK in vitro phosphorylation assay was performed. In RASK (KRAS) mutant cells acutely depleted of RAF1 (CRAF), ARAF and BRAF were immunoprecipitated and utilized to phosphorylate the MEK substrate via in vitro kinase assay. Immunoprecipitated ARAF phosphorylated MEK to similar levels as immunoprecipitated BRAF (FIG. 8D). Surprisingly, it was again observed that ARAF does not heterodimerize with BRAF, even in the absence of RAF1 (CRAF) (FIG. 8D). To further demonstrate that ARAF catalytic function is not dependent on BRAF activity upon RAF1 (CRAF) ablation, ARAF was immunoprecipitated from RAF1 (CRAF)-knockout and RAF1 (CRAF);BRAF double knockout RASK (KRAS) mutant cells. Although ARAF co-immunoprecipitated with RAF1 (CRAF) in the parental cells, RAF1 (CRAF) depletion did not promote ARAF and BRAF heterodimerization (FIG. 8E). This suggests that ARAF preferentially heterodimerizes with RAF1 (CRAF). However, mechanisms as to why ARAF does not dimerize with BRAF remains unclear. Additionally, ARAF homodimers function to activate MEK in the absence of both RAF1 (CRAF) and BRAF (FIG. 8E). This highlights an unprecedented catalytic function of ARAF homodimers in limiting growth of RASK (KRAS) mutant cells in the absence of RAF1 (CRAF).

### Example 6. Further Characterization of RAF1 (CRAF) Activity

a. ATP Assays

BRAF and RAF1 (CRAF) kinase activities have been shown to be highly regulated through numerous mechanisms that largely converge on dimerization. It has previously been shown that ATP binding to RAF1 (CRAF) and BRAF regulates RAF1 (CRAF):BRAF dimerization and kinase activity (Liau et al., Nat Struct Mol Biol, 27: 134-141, 2020). To determine whether the same is true for ARAF, similar kinase assays were conducted using recombinant purified RAF1 (CRAF):14-3-3, BRAF-14-3-3 and ARAF:14-3-3 dimers (FIG. 9D). Surprisingly, while both BRAF and RAF1 (CRAF) exhibited reduced kinase activity at high ATP concentrations, ARAF kinase activity was completely insensitive to elevated ATP concentrations. This effect mirrors what is seen for BRAF^{V600E}, which functions as a monomer and is insensitive to ATP concentrations.

b. Assessment of MAPK Signaling Under RAF1 (CRAF) Depletion

Taken together, disruption of RAF1 (CRAF):ARAF heterodimers enables ARAF catalytic activity to limit growth of RASK (KRAS) mutant cancer cells. It has previously been shown that sustained activation of MAPK signaling results in cell cycle arrest and/or differentiation (Nieto et al., Nature, 548: 239-243, 2017) and that cells have adopted multiple negative feedback loops to dampen pathway signaling in response to mitogenic stimuli (Unni et al., eLife, 7: e33718, 2018; Hanafusa et al., Nat Cell Biol, 4: 850-858, 2002; Kidger et al., Semin Cell Dev Biol, 50: 125-132, 2016).

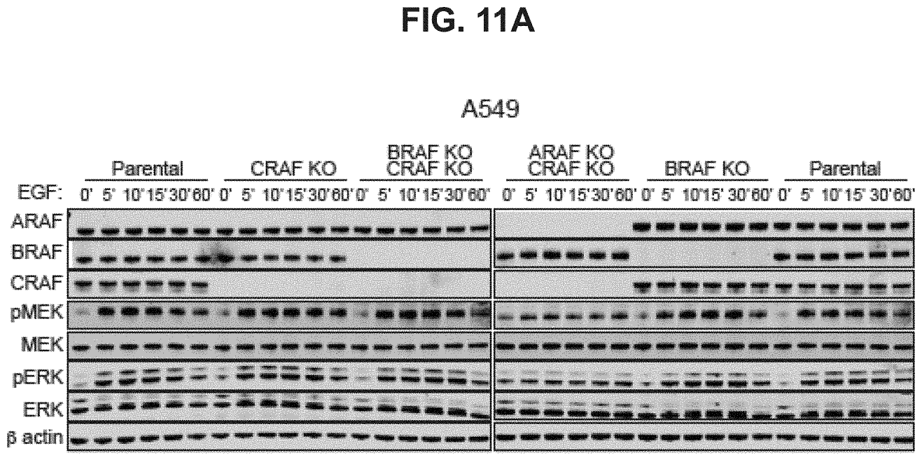

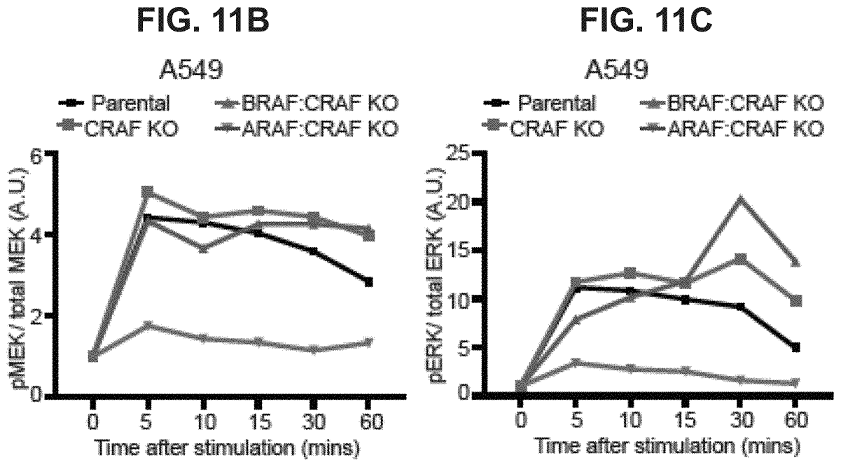

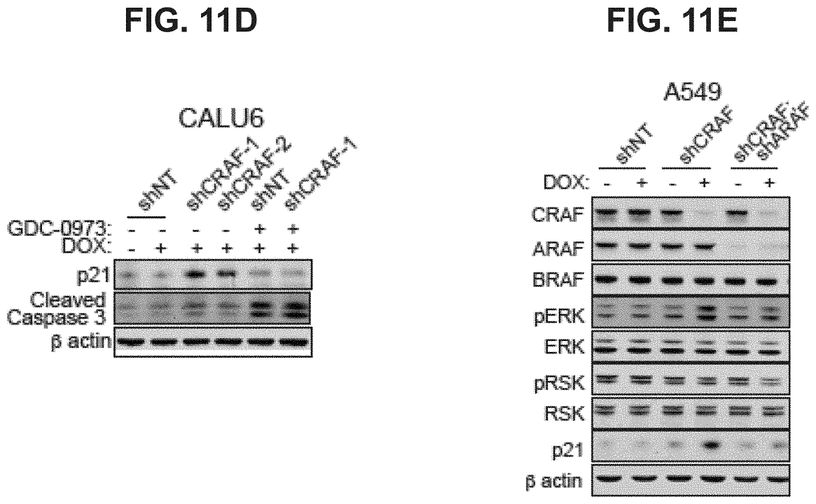

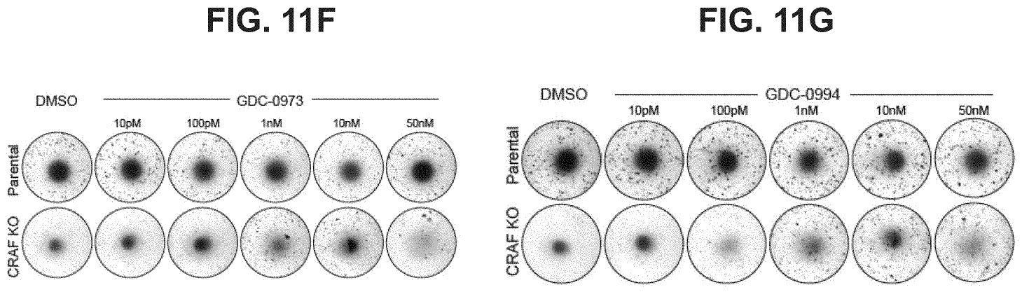

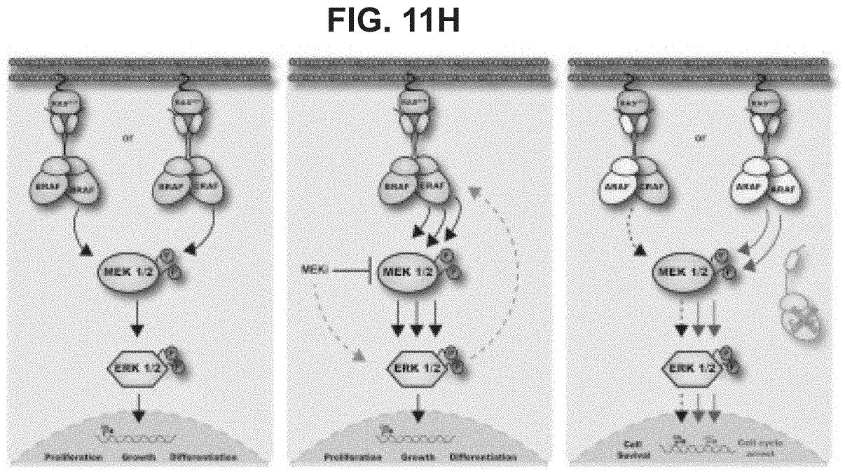

Indeed, activated ERK negatively regulates its upstream components through direct feedback phosphorylation of BRAF and RAF1 (CRAF), resulting in reduced RAF dimerization and kinase activity (Dougherty et al., Molecular Cell, 17: 215-224, 2005; Brummer et al., Oncogene, 22: 8823-8834, 2003; Ritt et al., Mol Cell Biol, 30: 806-819, 2010). It was hypothesized that upon depletion of RAF1 (CRAF), this negative regulatory mechanism would be lost and could result in tonic MAPK, over time driving cell cycle arrest. To determine whether RAF1 (CRAF) loss impacts the duration and amplitude of MAPK signaling, RAF CRISPR knockout cells were serum starved and stimulated with epidermal growth factor (EGF) and harvested over multiple time points. As expected, in parental cells, EGF stimulation caused robust induction of pERK within 5 minutes that was downregulated by 30 and 60 minutes, presumably due to the induction of negative feedback loops (FIGS. 11A-11C). In contrast, in both the RAF1 (CRAF) knockout and BRAF;RAF1 (CRAF) double knockout cells, MAPK activity was robust and sustained through 60 minutes (FIG. 11A). However, the ARAF;RAF1 (CRAF) double knockout cells promoted cell survival and exhibited a lower amplitude of MAPK activity, which may be more tolerable in RASK (KRAS) mutant cells (FIG. 11A). Consistent with our observation, we observed a sustained phosphorylation of MEK (FIG. 11B) and ERK (FIG. 11C) in the RAF1 (CRAF) knockout and BRAF;RAF1 (CRAF) double knockout cells upon EGF stimulation. This suggests that ARAF catalytic activity breaches a toxic threshold of MAPK pathway signaling which likely limits tumor cell growth. Conversely, ARAF;RAF1 (CRAF) double knockout cells exhibited a lower threshold of MAPK induction that directly correlated to the survival of RASK (KRAS) mutant cells (FIGS. 11B and 11C).

c. Assessment of Cell Death Under RAF1 (CRAF) Depletion

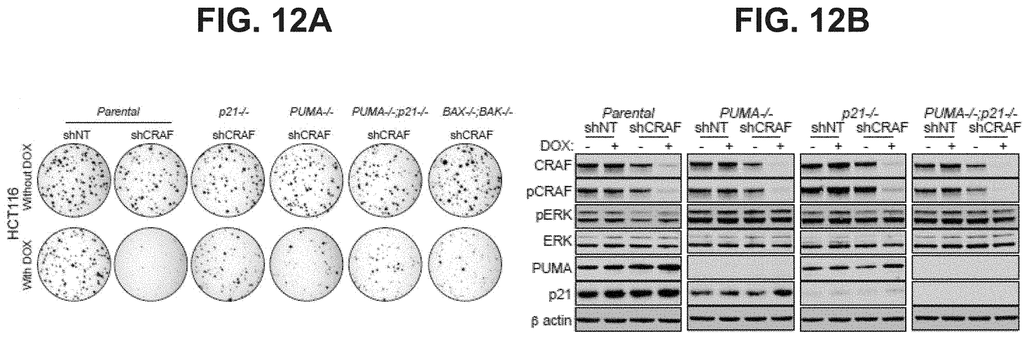

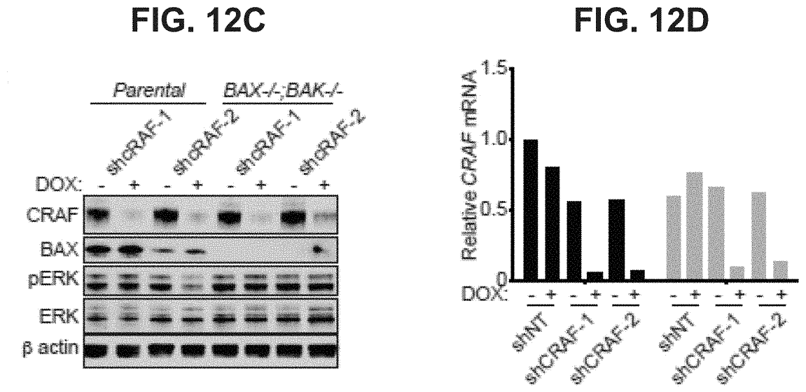

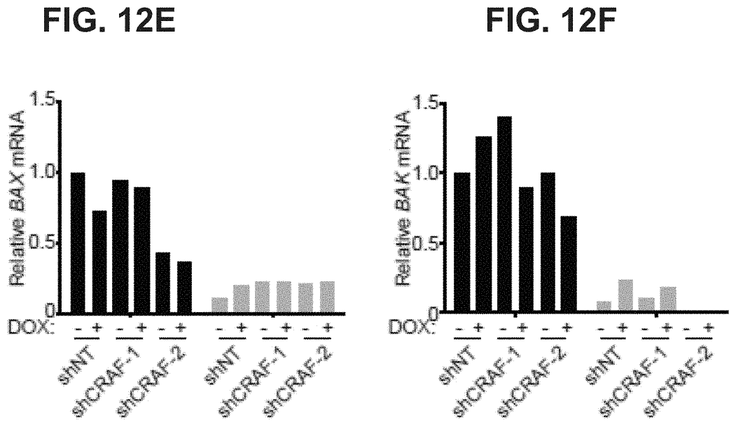

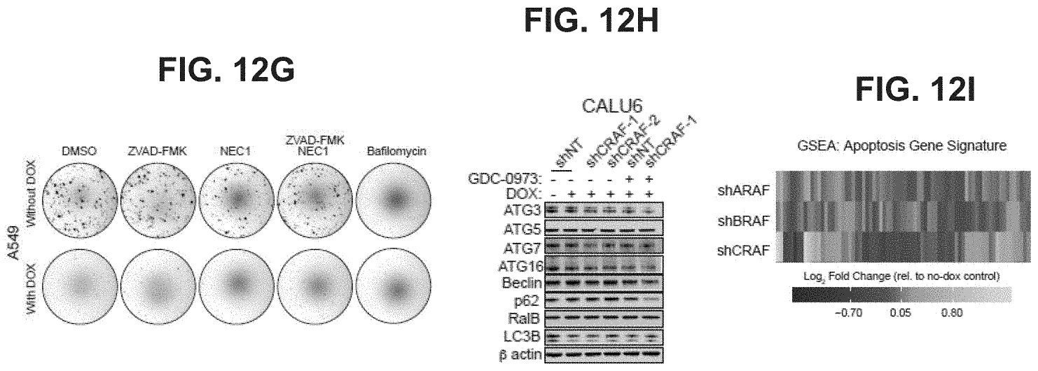

As ARAF catalytic function inhibits cell growth upon RAF1 (CRAF) loss, mechanisms by which ARAF limits growth of RASK (KRAS) mutant cells were investigated. Given that MAPK inhibition is associated with induction of cellular apoptosis (Yen et al., Cancer Cell, 34: 611-625, 2018), it was hypothesized that RAF1 (CRAF) ablation induces cell death, similar to MAPK inhibition in RASK (KRAS) mutants. Consistently, RAF1 (CRAF) ablation resulted in the induction of p21, a marker for cell-cycle arrest and not cellular apoptosis, compared to RASK (KRAS) mutant cells treated with a MEK inhibitor (GDC-0973) (FIG. 11D). Additionally, RAF1 (CRAF) ablation in a β21-knockout isogenic RASK (KRAS) mutant cell line (HCT116 p21^{-/-}) rescued cell growth inhibition mediated upon RAF1 (CRAF) loss (FIG. 12A). However, RAF1 (CRAF) ablation in PUMA knockout or a BAX:BAK double knockout RASK (KRAS) mutant cell lines did not rescue cell growth mediated upon RAF1 (CRAF) loss (FIGS. 12A-12F). A chemical inhibitor approach was adopted to test whether cell growth inhibition upon RAF1 (CRAF) loss could be rescued by the treatment with apoptosis or autophagic inhibitors. Treatment with either a pan-caspase inhibitor (Z-VAD-FMK) or a necroptosis inhibitor (NEC1) or a combination of both did not rescue cell growth in RAF1 (CRAF)-depleted RASK (KRAS) mutant cells (FIG. 12G). Separately, treatment with an autophagy inhibitor (bafilomycin) did not rescue cell growth in RAF1 (CRAF)-depleted RASK (KRAS) mutant cells (FIG. 12G). Additionally, RAF1 (CRAF) depletion and/or treatment of RASK (KRAS) mutant cells with a MEK inhibitor did not induce an autophagic response. Finally, a Gene Set Enrichment Analysis (GSEA) of targets involved in the apoptosis pathway was performed, but no significant enrichment of any apoptosis target gene set was observed upon acute depletion of ARAF, BRAF or RAF1 (CRAF) (FIG. 12I). These data show that RAF1 (CRAF) depletion in RASK (KRAS) mutant cancer cells induces cell cycle arrest and not cellular apoptosis. Thus, RAF1 (CRAF) depletion promotes p21-mediated cell cycle arrest in RASK (KRAS) mutant cancer cells. Further, co-depletion of RAF1 (CRAF) and ARAF in RASK (KRAS) mutant cells abrogated the expression levels of p21 (FIG. 4E). This suggests that ARAF catalytic function indirectly induces p21-mediated cell cycle arrest in RASK (KRAS) mutant cancer cells.

d. Treatment with a MEK Inhibitor

Previous reports have demonstrated that sustained oncogene-induced signaling is associated with DNA damage and tumor growth inhibition (Hills and Diffley, Curr Biol, 24: R435-444, 2014). Within the MAPK pathway, expression of BRAF^{D631A} mutation upon BRAF^{wild-type} loss induces cellular toxicity which impedes tumor development of RASK (KRAS) mutant lung tumors (Nieto et al., Nature, 548: 239-243, 2017; Yao et al., Nature, 548: 234-238, 2017). Importantly, pharmacological inhibition of ERK rescued tumor growth in this context (Nieto et al., Nature, 548: 239-243, 2017; Yao et al., Nature, 548: 234-238, 2017). To formally demonstrate that tonic ERK activation reduces tumor cell growth of RAF1 (CRAF)-depleted cells, the RAF1 (CRAF)-depleted cells were subjected to a low-dose treatment of MEK inhibitor (GDC-0973) (FIG. 11H) and an ERK inhibitor (GDC-0994) (FIG. 11I). Low-dose treatment of both of these drugs promoted cell survival in the RAF1 (CRAF) ablated RASK (KRAS) mutant cells, whereas, as expected, increasing the dose of either the MEK (GDC-0973) or ERK (GDC-0994) inhibitor in RAF1 (CRAF)-depleted cells limited cell survival. Thus, oncogenic cellular stress induced by ARAF catalytic activity could be inhibited by low-dose MAPK inhibition, thereby promoting survival of RASK (KRAS) mutant cells.

e. Conclusions

In this work, the kinase-dependent and kinase-independent roles of RAF1 (CRAF) in RASK (KRAS)-driven tumors are investigated. A subset of RASK (KRAS) and RASN (NRAS) mutant tumors were found to be dependent on RAF1 (CRAF) for growth. Interestingly, this dependence requires the RAF1 (CRAF) kinase domain, but not its catalytic activity. Mechanistic studies demonstrate that RAF1 (CRAF) dimerization with ARAF is required to maintain RASK (KRAS)-driven tumors, and that loss of RAF1 (CRAF) or disruption of RAF1 (CRAF) and ARAF heterodimer formation reduces tumor cell growth. Importantly, concurrent loss of ARAF with RAF1 (CRAF) rescues the lethality associated with RAF1 (CRAF) depletion. This study uncovers a potential new mechanism of regulation among RAF kinases through isoform-specific heterodimerization and its role in potentiating growth of mutant RASK (KRAS) cells. These data suggest that generation of RAF1 (CRAF) dimerization-specific inhibitors or isoform selective degradation may be efficacious in suppressing RASK (KRAS) mutant tumors.

Altogether, the results presented herein suggest that RAF1 (CRAF) dimerization preference regulates MAPK signaling in unique yet opposing ways, either through allosteric induction of kinase activity via BRAF:RAF1 (CRAF) dimers or through regulation of MAPK signaling duration and amplitude via RAF1 (CRAF):ARAF dimers. As such, RAS-driven tumorigenesis appears to critically depend on the stoichiometry of RAF homodimers and heterodimers in a cell- or tissue-specific manner. RAF homodimers and heterodimers are thus proposed to serve as a rheostat over MAPK signaling to avoid cell cycle arrest, senescence, or other tumor suppressive responses. From a therapeutic standpoint, selective disruption of RAF1 (CRAF)-containing dimers or chemical degradation of RAF1 (CRAF) may be more beneficial in RASK (KRAS)-driven tumors than pan-RAF kinase inhibition alone.

### Example 7. Determination of Protein Levels of Members of the PI3K Pathway

In another example, a protein level of one or more members of the PI3K pathway (e.g., P85A, P85B, PK3CA (also called PIK3CA and p110-alpha), PK3CA^{E545K}, PK3CA^{H1047K}, PK3CD, PK3CB, ERBB2 (HER2), EGFR, RRAS2, and P55G) is determined in a sample from a subject using the methods described herein.

An exemplary method includes (a) adding a recombinant protein described in Section IIC herein to a sample from the subject, the recombinant protein comprising at least one polypeptide corresponding to each protein for which a level is to be determined; (b) exposing the sample to a cleavage agent (e.g., trypsin) that cleaves the recombinant protein and proteins from the sample, (c) measuring a level of one or more internal standard polypeptides derived from the recombinant protein and a level of one or more corresponding polypeptides from the sample; and (d) comparing the level of the one or more internal standard polypeptides to the level of the one or more corresponding polypeptides from the sample, thereby determining a protein level of one or more of P85A, P85B, PK3CA, PK3CA^{E545K}, PK3CA^{H1047K}PK3CD, PK3CB, ERBB2, EGFR, RRAS2, and P55G in the sample.

In one example, the recombinant protein includes a set of polypeptides having the amino acid sequences of SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 82, SEQ ID NO: 83, SEQ ID NO: 84, SEQ ID NO: 85, SEQ ID NO: 86, and SEQ ID NO: 87. The recombinant protein may further include polypeptides having the amino acid sequences of SEQ ID NO: 76, SEQ ID NO: 77, SEQ ID NO: 78, SEQ ID NO: 79, SEQ ID NO: 80, and/or SEQ ID NO: 81.

In one example, the recombinant protein consists of the amino acid sequence of SEQ ID NO: 44).

An exemplary method uses a PIKES approach as described in Example 3, above.

### Example 7. Quality Control Techniques for QCONCAT Polypeptides

The QCONCAT construct described in Example 3 was tested to verify utility. The construct was subjected to reduction, alkylation, and trypsin digestion followed by LC-MS/MS to confirm that under typical sample handling conditions (e.g., conditions as described in Example 3(c)), tryptic peptides of interest were produced as anticipated. Additionally, heavy isotope incorporation for each tryptic peptide was examined to ensure that incorporation of the heavy label was 98%.

What is claimed is:

1. A recombinant protein comprising a set of non-identical, contiguous polypeptides, the set comprising:
a polypeptide consisting of a sequence present in RAF1;
a polypeptide consisting of a sequence present in BRAF;
a polypeptide consisting of a sequence present in BRAF^{V600E};
a polypeptide consisting of a sequence present in ARAF;
a polypeptide consisting of a sequence present in MP2K1;
a polypeptide consisting of a sequence present in MP2K2;
a polypeptide consisting of a sequence present in MK03;
a polypeptide consisting of a sequence present in MK01;
a polypeptide consisting of a sequence present in RASK;
a polypeptide consisting of a sequence present in RASN;
a polypeptide consisting of a sequence present in RASH;
a polypeptide consisting of a sequence present in each of RAF1, BRAF, and ARAF;
a polypeptide consisting of a sequence present in both of RASH and RASN;
a polypeptide consisting of a sequence present in both of RASN and RASK;
a polypeptide consisting of a sequence present in each of RASH, RASN, and RASK;
a polypeptide consisting of a sequence present in each of RASH^{Q61K}, RASN^{Q61K}, and RASK^{Q61K};
a polypeptide consisting of a sequence present in each of RASH^{Q61R}, RASN^{Q61R}, and RASK^{Q61R};
a polypeptide consisting of a sequence present in each of RASH^{G12V}, RASN^{G12V}, and RASK^{G12V};
a polypeptide consisting of a sequence present in each of RASH^{G13D}, RASN^{G13D}, and RASK^{G13D};
a polypeptide consisting of a sequence present in each of RASH^{G12C}, RASN^{G12C}, and RASK^{G12C};
a polypeptide consisting of a sequence present in each of RASH^{G12D}, RASN^{G12D}, and RASK^{G12D}; and
a polypeptide consisting of a sequence present in each of RASH^{G12S}, RASN^{G12S}, and RASK^{G12S};
wherein the recombinant protein comprises a trypsin cleavage site between each polypeptide of the set that allows separation of each polypeptide upon exposure of the recombinant protein to trypsin.

2. The recombinant protein of claim 1, wherein each of the polypeptides is between 6 and 25 amino acid residues in length.

3. The recombinant protein of claim 1 or 2, wherein:
the polypeptide consisting of a sequence present in BRAF^{V600E} has the amino acid sequence of SEQ ID NO: 10;
the polypeptide consisting of a sequence present in each of RASH^{Q61K}, RASN^{Q61K}, and RASK^{Q61K} has the amino acid sequence of SEQ ID NO: 37;
the polypeptide consisting of a sequence present in each of RASH^{Q61R}, RASN^{Q61R}, and RASK^{Q61R} has the amino acid sequence of SEQ ID NO: 38;
the polypeptide consisting of a sequence present in each of RASH^{G12V}, RASN^{G12V}, and RASK^{G12V} has the amino acid sequence of SEQ ID NO: 39;
the polypeptide consisting of a sequence present in each of RASH^{G13D}, RASN^{G13D}, and RASK^{G13D} has the amino acid sequence of SEQ ID NO: 40;
the polypeptide consisting of a sequence present in each of RASH^{G12C}, RASN^{G12C}, and RASK^{G12C} has the amino acid sequence of SEQ ID NO: 41;
the polypeptide consisting of a sequence present in each of RASH^{G12D}, RASN^{G12D}, and RASK^{G12D} has the amino acid sequence of SEQ ID NO: 42; and/or
the polypeptide consisting of a sequence present in each of RASH^{G12S}, RASN^{G12S}, and RASK^{G12S} has the amino acid sequence of SEQ ID NO: 43.

4. The recombinant protein of any one of claims 1-3, wherein the set comprises at least two polypeptides consisting of a sequence present in RAF1, BRAF, ARAF, MP2K1, MP2K2, MK03, MK01, RASK, RASN, or RASH; at least two polypeptides consisting of a sequence present in both of RASH and RASN; and/or at least two polypeptides consisting of a sequence present in each of RASH, RASN, and RASK.

5. The recombinant protein of any one of claims 1-4, wherein the set further comprises one or more polypeptides consisting of a sequence present in one or more additional target molecules, and wherein the recombinant protein comprises a trypsin cleavage site between each of the one or more polypeptides that allows separation of each polypeptide upon exposure of the recombinant protein to trypsin.

6. A recombinant protein comprising a set of polypeptides having the amino acid sequences of SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, and SEQ ID NO: 43.

7. The recombinant protein of any one of claims 1-6, further comprising an N-terminal sequence comprising methionine and a trypsin cleavage site between the N-terminal sequence and the set of polypeptides that allows separation of the N-terminal sequence from the set of polypeptides upon exposure of the recombinant protein to trypsin.

8. The recombinant protein of claim 7, wherein the N-terminal sequence has the amino acid sequence of SEQ ID NO: 88.

9. The recombinant protein of any one of claims 1-8, further comprising a C-terminal sequence comprising a tag and a trypsin cleavage site between the C-terminal sequence and the set of polypeptides that allows separation of the C-terminal sequence from the set of polypeptides upon exposure of the recombinant protein to trypsin.

10. The recombinant protein of claim 9, wherein the tag is a polyhistidine tag.

11. The recombinant protein of claim 10, wherein the C-terminal sequence has the amino acid sequence of SEQ ID NO: 89.

12. The recombinant protein of claim 9, wherein the tag is a FLAG tag or a V5 tag.

13. The recombinant protein of any one of claims 1-4 and 6-11, wherein the recombinant protein comprises the amino acid sequence of SEQ ID NO: 1.

14. A recombinant protein consisting of the amino acid sequence of SEQ ID NO: 1.

15. The recombinant protein of any one of claims 1-14, wherein each polypeptide of the set comprises a label.

16. The recombinant protein of claim 15, wherein the label is an isotopic label.

17. The recombinant protein of claim 16, wherein the isotopic label is heavy arginine.

18. The recombinant protein of claim 17, wherein the heavy arginine is ¹³C₁-arginine (R1); ¹³C₂-arginine (R2); ¹⁵N₄-arginine (R4); ¹³C₆-arginine (R6); ²H₇-arginine (R7); ¹³C₆, ¹⁵N₄-arginine (R10); ²H₇, ¹⁵N₄-arginine (R11), or ¹³C₆, ²H₇, ¹⁵N₄-arginine (R17).

19. The recombinant protein of claim 16, wherein the isotopic label is heavy lysine.

20. The recombinant protein of claim 19, wherein the heavy lysine is, ¹³C₁-lysine (K1); ¹⁵N₂-lysine (K2); ²H₄-lysine (K4); ¹³C₆-lysine (K6); ¹³C₆, ¹⁵N₂-lysine (K8); ²H₈-lysine (K8); ²H₉-lysine (K9); ²H₉, ¹⁵N₂-lysine (K11); or ¹³C₆; ²H₉, ¹⁵N₂-lysine (K17).

21. The recombinant protein of claim 15, wherein the label is a chemical label.

22. The recombinant protein of claim 21, wherein the chemical label is a tandem mass tag (TMT), an iTRAQ, a label produced by reductive methylation/dimethylation, or a label produced by acetylation.

23. The recombinant protein of any one of claims 15-22, wherein the recombinant protein is at least 98% labeled.

24. The recombinant protein of claim 23, wherein the recombinant protein is at least 99% labeled.

25. A method for determining a protein level in a sample from a subject of one or more of RAF1, BRAF, BRAF^{V600E}, ARAF, MP2K1, MP2K2, MK03, MK01, RASK, RASN, RASH; RASH and RASN; RASN and RASK; RASH, RASN, and RASK; RASH^{Q61K}, RASN^{Q61K}, and RASK^{Q61K}; RASH^{Q61R}, RASN^{Q61R}, and RASK^{Q61R}; RASH^{G12V}, RASN^{G12V}, and RASK^{G12V}; RASH^{G13D}, RASN^{G13D}, and RASK^{G13D}; RASH^{G12C}, RASN^{G12C}, and RASK^{G12C}; RASH^{G12D}, RASN^{G12D}, and RASK^{G12D}; and RASH^{G12S}, RASN^{G12S}, and RASK^{G12S};
the method comprising:
(a) adding to the sample an amount of the recombinant protein of any one of claims 1-24;
(b) exposing the sample following step (a) to trypsin, whereby the recombinant protein is cleaved, thereby generating an equimolar set of internal standard polypeptides, the set comprising:
a polypeptide consisting of a sequence present in RAF1;
a polypeptide consisting of a sequence present in BRAF;
a polypeptide consisting of a sequence present in BRAF^{V600E};
a polypeptide consisting of a sequence present in ARAF;
a polypeptide consisting of a sequence present in MP2K1;
a polypeptide consisting of a sequence present in MP2K2;
a polypeptide consisting of a sequence present in MK03;
a polypeptide consisting of a sequence present in MK01;
a polypeptide consisting of a sequence present in RASK;
a polypeptide consisting of a sequence present in RASN;
a polypeptide consisting of a sequence present in RASH;
a polypeptide consisting of a sequence present in each of RAF1, BRAF, and ARAF;
a polypeptide consisting of a sequence present in both of RASH and RASN;
a polypeptide consisting of a sequence present in both of RASN and RASK;
a polypeptide consisting of a sequence present in each of RASH, RASN, and RASK;
a polypeptide consisting of a sequence present in each of RASH^{Q61K}, RASN^{Q61K}, and RASK^{Q61K};
a polypeptide consisting of a sequence present in each of RASH^{Q61R}, RASN^{Q61R}, and RASK^{Q61R};
a polypeptide consisting of a sequence present in each of RASH^{G12V}, RASN^{G12V}, and RASK^{G12V};
a polypeptide consisting of a sequence present in each of RASH^{G13D}, RASN^{G13D}, and RASK^{G13D};
a polypeptide consisting of a sequence present in each of RASH^{G12C}, RASN^{G12C}, and RASK^{G12C};
a polypeptide consisting of a sequence present in each of RASH^{G12D}, RASN^{G12D}, and RASK^{G12D}; and
a polypeptide consisting of a sequence present in each of RASH^{G12S}, RASN^{G12S}, and RASK^{G12S};
(c) measuring a level of one or more internal standard polypeptides and a level of one or more corresponding polypeptides from the sample; and
(d) comparing the level of the one or more internal standard polypeptides and the level of the one or more corresponding polypeptides from the sample, thereby determining a protein level of one or more of:
RAF1, BRAF, BRAF^{V600E}, ARAF, MP2K1, MP2K2, MK03, MK01, RASK, RASN, RASH; RASH and RASN; RASN and RASK; RASH, RASN, and RASK; RASH^{Q61K}, RASN^{Q61K}, and RASK^{Q61K}; RASH^{Q61R}, RASN^{Q61R}, and RASK^{Q61R}; RASH^{G12V}, RASN^{G12V}, and RASK^{G12V}; RASH^{G13D}, RASN^{G13D}, and RASK^{G13D}; RASH^{G12C}, RASN^{G12C}, and RASK^{G12C}; RASH^{G12D}, RASN^{G12D}, and RASK^{G12D}; and RASH^{G12S}, RASN^{G12S}, and RASK^{G12S} in the sample.

26. The method of claim 25, comprising determining a protein level of one or more of RASH, RASN, RASK, ARAF, BRAF, and RAF1 in the sample.

27. The method of claim 26, comprising determining a protein level of each of RASH, RASN, RASK, ARAF, BRAF, and RAF1 in the sample.

28. The method of any one of claims 25-27, wherein the protein level is a relative protein level.

29. The method of any one of claims 25-27, wherein the protein level is an absolute protein level.

30. The method of any one of claims 25-27, wherein the method is performed for at least two samples from the subject.

31. The method of claim 30, wherein the at least two samples are from at least two different time points.

32. The method of claim 31, wherein the at least two different time points include a time point before administration of an agent to the subject and a timepoint after administration of the agent to the subject.

33. The method of any one of claims 25-32, wherein the measuring of step (c) comprises mass spectrometry (MS).

34. The method of claim 33, wherein the MS is parallel reaction monitoring MS (PRM-MS).

35. The method of any one of claims 25-34, wherein the sample is a human sample.

36. The method of any one of claims 25-35, wherein the sample is a tumor sample.

37. The method of any one of claims 25-36, wherein the sample is a lysate.

38. The method of any one of claims 25-37, wherein the sample is an immunoprecipitate of a target protein.

39. The method of claim 38, wherein the method comprises determining the ratio of the target protein to one or more of RAF1, BRAF, BRAF^{V600E}, ARAF, MP2K1, MP2K2, MK03, MK01, RASK, RASN, RASH; RASH and RASN; RASN and RASK; RASH, RASN, and RASK; RASH^{Q61K}, RASN^{Q61K}, and RASK^{Q61K}; RASH^{Q61R}, RASN^{Q61R}, and RASK^{Q61R}; RASH^{G12V}, RASN^{G12V}, and RASK^{G12V}; RASH^{G13D}, RASN^{G13D}, and RASK^{G13D}; RASH^{G12C}, RASN^{G12C}, and RASK^{G12C}; RASH^{G12D}, RASN^{G12D}, and RASK^{G12D}; and RASH^{G12S}, RASN^{G12S}, and RASK^{G12S}.

40. A nucleic acid encoding the recombinant protein of any one of claims 1-24.

41. A recombinant protein comprising a set of non-identical, contiguous polypeptides, the set comprising:
a polypeptide consisting of a sequence present in P85A;
a polypeptide consisting of a sequence present in P85B;
a polypeptide consisting of a sequence present in PK3CA;
a polypeptide consisting of a sequence present in PK3CA^{E545K};
a polypeptide consisting of a sequence present in PK3CA^{H1047K};
a polypeptide consisting of a sequence present in PK3CD;
a polypeptide consisting of a sequence present in PK3CB;
a polypeptide consisting of a sequence present in ERBB2;
a polypeptide consisting of a sequence present in EGFR;
a polypeptide consisting of a sequence present in RRAS2; and
a polypeptide consisting of a sequence present in P55G,
wherein the recombinant protein comprises a trypsin cleavage site between each polypeptide of the set that allows separation of each polypeptide upon exposure of the recombinant protein to trypsin.

42. The recombinant protein of claim 41, wherein each of the polypeptides is between 6 and 25 amino acid residues in length.

43. The recombinant protein of claim 41 or 42, wherein the polypeptide consisting of a sequence present in PK3CA^{E545K} has the amino acid sequence of SEQ ID NO: 56 and/or the polypeptide consisting of a sequence present in PK3CA^{H1047K} has the amino acid sequence of SEQ ID NO: 58 or SEQ ID NO: 59.

44. The recombinant protein of any one of claims 41-43, wherein the set comprises at least two polypeptides consisting of a sequence present in P85A, P85B, PK3CA, PK3CA^{E545K}, PK3CA^{H1047K}, PK3CD, PK3CB, ERBB2, EGFR, RRAS2, or P55G.

45. The recombinant protein of any one of claims 41-44, further comprising a polypeptide consisting of a sequence present in a control protein.

46. The recombinant protein of claim 45, wherein the control protein is G3P or ACTA.

47. The recombinant protein of claim 46, wherein the recombinant protein comprises a polypeptide consisting of a sequence present in G3P and a polypeptide consisting of a sequence present in ACTA.

48. The recombinant protein of any one of claims 45-47, wherein the set comprises at least two polypeptides consisting of a sequence present in G3P or ACTA.

49. The recombinant protein of any one of claims 41-48, further comprising one or more additional non-identical, contiguous polypeptides consisting of a sequence present in one or more additional target molecules, wherein each of the one or more additional polypeptides comprises a cleavage site that allows separation of the polypeptide from the set upon exposure of the recombinant protein to trypsin.

50. A recombinant protein comprising a set of polypeptides having the amino acid sequences of SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 82, SEQ ID NO: 83, SEQ ID NO: 84, SEQ ID NO: 85, SEQ ID NO: 86, and SEQ ID NO: 87.

51. The recombinant protein of claim 50, wherein the set of polypeptides further comprises polypeptides having the amino acid sequences of SEQ ID NO: 76, SEQ ID NO: 77, SEQ ID NO: 78, SEQ ID NO: 79, SEQ ID NO: 80, and SEQ ID NO: 81.

52. The recombinant protein of any one of claims 41-51, further comprising an N-terminal sequence comprising methionine and a trypsin cleavage site between the N-terminal sequence and the set of polypeptides that allows separation of the N-terminal sequence from the set of polypeptides upon exposure of the recombinant protein to trypsin.

53. The recombinant protein of claim 52, wherein the N-terminal sequence has the amino acid sequence of SEQ ID NO: 88.

54. The recombinant protein of any one of claims 41-53, further comprising a C-terminal sequence comprising a tag and a trypsin cleavage site between the C-terminal sequence and the set of polypeptides that allows separation of the C-terminal sequence from the set of polypeptides upon exposure of the recombinant protein to trypsin.

55. The recombinant protein of claim 54, wherein the tag is a polyhistidine tag.

56. The recombinant protein of claim 55, wherein the C-terminal sequence has the amino acid sequence of SEQ ID NO: 89.

57. The recombinant protein of claim 54, wherein the tag is a FLAG tag or a V5 tag.

58. The recombinant protein of any one of claims 41-48 and 51-56, wherein the recombinant protein comprises the amino acid sequence of SEQ ID NO: 44.

59. A recombinant protein consisting of the amino acid sequence of SEQ ID NO: 44.

60. The recombinant protein of any one of claims 41-59, wherein each polypeptide of the set comprises a label.

61. The recombinant protein of claim 60, wherein the label is an isotopic label.

62. The recombinant protein of claim 61, wherein the isotopic label is heavy arginine.

63. The recombinant protein of claim 62, wherein the heavy arginine is ¹³C₁-arginine (R1); ¹³C₂-arginine (R2); ¹⁵N₄-arginine (R4); ¹³C₆-arginine (R6); ²H₇-arginine (R7); ¹³C₆, ¹⁵N₄-arginine (R10); ²H₇, ¹⁵N₄-arginine (R11), or ¹³C₆, ²H₇, ¹⁵N₄-arginine (R17).

64. The recombinant protein of claim 61, wherein the isotopic label is heavy lysine.

65. The recombinant protein of claim 64, wherein the heavy lysine is ¹³C₁-lysine (K1); ¹⁵N₂-lysine (K2); ²H₄-lysine (K4); ¹³C₆-lysine (K6); ¹³C₆, ¹⁵N₂-lysine (K8); ²H₈-lysine (K8); ²H₉-lysine (K9); ²H₉, ¹⁵N₂-lysine (K11); or ¹³C₆; ²H₉, ¹⁵N₂-lysine (K17).

66. The recombinant protein of claim 60, wherein the label is a chemical label.

67. The recombinant protein of claim 66, wherein the chemical label is a tandem mass tag (TMT), an iTRAQ, a label produced by reductive methylation/dimethylation, or a label produced by acetylation.

68. The recombinant protein of any one of claims 60-67, wherein the recombinant protein is at least 98% labeled.

69. The recombinant protein of claim 68, wherein the recombinant protein is at least 99% labeled.

70. A method for determining a protein level in a sample from a subject of one or more of P85A, P85B, PK3CA, PK3CA^{E545K}, PK3CA^{H1047K}, PK3CD, PK3CB, ERBB2, EGFR, RRAS2, and P55G;
the method comprising:
(a) adding to the sample an amount of the recombinant protein of any one of claims 41-69;
(b) exposing the sample following step (a) to trypsin, whereby the recombinant protein is cleaved, thereby generating an equimolar set of internal standard polypeptides, the set comprising:
a polypeptide consisting of a sequence present in P85A;
a polypeptide consisting of a sequence present in P85B;
a polypeptide consisting of a sequence present in PK3CA;
a polypeptide consisting of a sequence present in PK3CA^{E545K};
a polypeptide consisting of a sequence present in PK3CA^{H1047K};
a polypeptide consisting of a sequence present in PK3CD;
a polypeptide consisting of a sequence present in PK3CB;
a polypeptide consisting of a sequence present in ERBB2;
a polypeptide consisting of a sequence present in EGFR;
a polypeptide consisting of a sequence present in RRAS2;
and a polypeptide consisting of a sequence present in P55G;
(c) measuring a level of one or more internal standard polypeptides and a level of one or more corresponding polypeptides from the sample; and
(d) comparing the level of the one or more internal standard polypeptides and the level of the one or more corresponding polypeptides from the sample, thereby determining a protein level of one or more of P85A, P85B, PK3CA, PK3CA^{E545K}, PK3CA^{H1047K}, PK3CD, PK3CB, ERBB2, EGFR, RRAS2, and P55G in the sample.

71. The method of claim 70, further comprising determining a protein level of G3P and/or ACTA in the sample from the subject, wherein the set of internal standard polypeptides of step (b) comprises a polypeptide consisting of a sequence present in G3P and/or a polypeptide consisting of a sequence present in ACTA.

72. The method of claim 70 or 71, wherein the protein level is a relative protein level.

73. The method of claim 70 or 71, wherein the protein level is an absolute protein level.

74. The method of claim 70 or 71, wherein the method is performed for at least two samples from the subject.

75. The method of claim 74, wherein the at least two samples are from at least two different time points.

76. The method of claim 75, wherein the at least two different time points include a time point before administration of an agent to the subject and a timepoint after administration of the agent to the subject.

77. The method of any one of claims 70-76, wherein the measuring of step (c) comprises mass spectrometry (MS).

78. The method of claim 77, wherein the MS is parallel reaction monitoring MS (PRM-MS).

79. The method of any one of claims 70-78, wherein the sample is a human sample.

80. The method of any one of claims 70-79, wherein the sample is a tumor sample.

81. The method of any one of claims 70-80, wherein the sample is a lysate.

82. The method of any one of claims 70-81, wherein the sample is an immunoprecipitate of a target protein.

83. The method of claim 82, wherein the method comprises determining the ratio of the target protein to one or more of P85A, P85B, PK3CA, PK3CA^{E545K}, PK3CA^{H1047K}, PK3CD, PK3CB, ERBB2, EGFR, G3P, ACTA, RRAS2, and P55G.

84. A nucleic acid encoding the recombinant protein of any one of claims 41-69.


SEQUENCE LISTING (102 sequences)

SEQ ID NO: 1 - protein, 542 aa, synthetic construct
MAGRVVDPTPEQFQAFRDNLAIVTQWCEGSSLYKSNNIFLHEDLTVKLTQEHIEALLDKSTSTPNVHMVSTTLPVDSRMLNVTAPTPQQLQAFKMLTPQQLQAFKIGDFGLATVKIGDFGLATEKVSQPTAEQAQAFKLVVVGADGVGKIGTGSFGTVFRFDMVQLIDVARGQPFDVGPRIGDFGLATVKLPSGVFSLEFQDFVNKISELGAGNGGVVFKIPEQILGKISELGAGNGGVVTKLPNGVFTPDFQEFVNKLNQPGTPTRLVVVGAVGVGKSQQLSNDHICYFLYQILRNYLLSLPHKGQVFDVGPRHENIIGINDIIRSFEDIHHYRQVVIDGETCLLDILDTAGRDSEDVPMVLVGNKSFADINLYRLVVVGACGVGKQAHELAKSFEDIHQYRLVVVGAGDVGKSYGIPYIETSAKDSDDVPMVLVGNKQGVEDAFYTLVRSYGIPFIETSAKLVVVGAGGVGKQVVIDGETCLLDILDTAGQEEYSAMRLVVVGASGVGKQVVIDGETCLLDILDTAGKLAAALEHHHHHH

SEQ ID NO: 2 - protein, 13 aa, synthetic construct
VVDPTPEQFQAFR

SEQ ID NO: 3 - protein, 17 aa, synthetic construct
DNLAIVTQWCEGSSLYK

SEQ ID NO: 4 - protein, 19 aa, synthetic construct
STSTPNVHMVSTTLPVDSR

SEQ ID NO: 5 - protein, 13 aa, synthetic construct
SNNIFLHEDLTVK

SEQ ID NO: 6 - protein, 12 aa, synthetic construct
LTQEHIEALLDK

SEQ ID NO: 7 - protein, 16 aa, synthetic construct
MLNVTAPTPQQLQAFK

SEQ ID NO: 8 - protein, 11 aa, synthetic construct
MLTPQQLQAFK

SEQ ID NO: 9 - protein, 10 aa, synthetic construct
IGDFGLATVK

SEQ ID NO: 10 - protein, 10 aa, synthetic construct
IGDFGLATEK

SEQ ID NO: 11 - protein, 13 aa, synthetic construct
VSQPTAEQAQAFK

SEQ ID NO: 12 - protein, 11 aa, synthetic construct
IGTGSFGTVFR

SEQ ID NO: 13 - protein, 11 aa, synthetic construct
FDMVQLIDVAR

SEQ ID NO: 14 - protein, 10 aa, synthetic construct
IGDFGLATVK

SEQ ID NO: 15 - protein, 16 aa, synthetic construct
LPSGVFSLEFQDFVNK

SEQ ID NO: 16 - protein, 14 aa, synthetic construct
ISELGAGNGGVVFK

SEQ ID NO: 17 - protein, 8 aa, synthetic construct
IPEQILGK

SEQ ID NO: 18 - protein, 14 aa, synthetic construct
ISELGAGNGGVVTK

SEQ ID NO: 19 - protein, 16 aa, synthetic construct
LPNGVFTPDFQEFVNK

SEQ ID NO: 20 - protein, 9 aa, synthetic construct
LNQPGTPTR

SEQ ID NO: 21 - protein, 9 aa, synthetic construct
GQPFDVGPR

SEQ ID NO: 22 - protein, 18 aa, synthetic construct
SQQLSNDHICYFLYQILR

SEQ ID NO: 23 - protein, 9 aa, synthetic construct
NYLLSLPHK

SEQ ID NO: 24 - protein, 9 aa, synthetic construct
GQVFDVGPR

SEQ ID NO: 25 - protein, 12 aa, synthetic construct
HENIIGINDIIR

SEQ ID NO: 26 - protein, 9 aa, synthetic construct
SFEDIHHYR

SEQ ID NO: 27 - protein, 13 aa, synthetic construct
DSEDVPMVLVGNK

SEQ ID NO: 28 - protein, 9 aa, synthetic construct
SFADINLYR

SEQ ID NO: 29 - protein, 7 aa, synthetic construct
QAHELAK

SEQ ID NO: 30 - protein, 9 aa, synthetic construct
SFEDIHQYR

SEQ ID NO: 31 - protein, 12 aa, synthetic construct
SYGIPYIETSAK

SEQ ID NO: 32 - protein, 13 aa, synthetic construct
DSDDVPMVLVGNK

SEQ ID NO: 33 - protein, 12 aa, synthetic construct
SYGIPFIETSAK

SEQ ID NO: 34 - protein, 12 aa, synthetic construct
QGVEDAFYTLVR

SEQ ID NO: 35 - protein, 11 aa, synthetic construct
LVVVGAGGVGK

SEQ ID NO: 36 - protein, 26 aa, synthetic construct
QVVIDGETCLLDILDTAGQEEYSAMR

SEQ ID NO: 37 - protein, 19 aa, synthetic construct
QVVIDGETCLLDILDTAGK

SEQ ID NO: 38 - protein, 19 aa, synthetic construct
QVVIDGETCLLDILDTAGR

SEQ ID NO: 39 - protein, 11 aa, synthetic construct
LVVVGAVGVGK

SEQ ID NO: 40 - protein, 11 aa, synthetic construct
LVVVGAGDVGK

SEQ ID NO: 41 - protein, 11 aa, synthetic construct
LVVVGACGVGK

SEQ ID NO: 42 - protein, 11 aa, synthetic construct
LVVVGADGVGK

SEQ ID NO: 43 - protein, 11 aa, synthetic construct
LVVVGASGVGK

SEQ ID NO: 44 - protein, 499 aa, synthetic construct
MAGRTQSSSNLAELRNESLAQYNPKAALQALGVAEGGERLDILDTAGQEEFGAMRIQGEYTLTLREAGFSYSHAGLSNRYEQYLDNLLVREIFPQSLPKEATLITIKDLNSPHSRYLVIQGDERDPLSEITKQMNDARHGGWTTKTGLIEVVLRTDLHAFENLEIIRGELLNPTGTVRIELIQGSKHEVQEHFPEALARNANLSTIKAAEIASSDSANVSSREATISYIKVFGEDSVGVIFKEYNSGDDLDRVLQGLPRGLQSLPTHDPSPLQRFVVIQNEDLGPASPLDSTFYRIPLENLQIIRLQEYHSQYQEKALGATFGPLLLRDPLSEITEQEKLVINGNPITIFQERGALQNIIPASTGAAKVGVNGFGRAGFAGDDAPRAVFPSIVGRPRSYELPDGQVITIGNERMNVDQAFHELVRFSAASSDNTENLIKHCVIYSTARQMNDAHHGGWTTKVQAEDLLYGKPDGAFLIRGSFEEIYKLAAALEHHHHHH

SEQ ID NO: 45 - protein, 14 aa, synthetic construct
FSAASSDNTENLIK

SEQ ID NO: 46 - protein, 11 aa, synthetic construct
TQSSSNLAELR

SEQ ID NO: 47 - protein, 10 aa, synthetic construct
NESLAQYNPK

SEQ ID NO: 48 - protein, 14 aa, synthetic construct
AALQALGVAEGGER

SEQ ID NO: 49 - protein, 12 aa, synthetic construct
ALGATFGPLLLR

SEQ ID NO: 50 - protein, 11 aa, synthetic construct
APGPGPPPAAR

SEQ ID NO: 51 - protein, 14 aa, synthetic construct
EAGFSYSHAGLSNR

SEQ ID NO: 52 - protein, 11 aa, synthetic construct
YEQYLDNLLVR

SEQ ID NO: 53 - protein, 8 aa, synthetic construct
EATLITIK

SEQ ID NO: 54 - protein, 8 aa, synthetic construct
DLNSPHSR

SEQ ID NO: 55 - protein, 11 aa, synthetic construct
DPLSEITEQEK

SEQ ID NO: 56 - protein, 8 aa, synthetic construct
DPLSEITK

SEQ ID NO: 57 - protein, 13 aa, synthetic construct
QMNDAHHGGWTTK

SEQ ID NO: 58 - protein, 6 aa, synthetic construct
QMNDAR

SEQ ID NO: 59 - protein, 7 aa, synthetic construct
HGGWTTK

SEQ ID NO: 60 - protein, 9 aa, synthetic construct
TGLIEVVLR

SEQ ID NO: 61 - protein, 11 aa, synthetic construct
GELLNPTGTVR

SEQ ID NO: 62 - protein, 8 aa, synthetic construct
IELIQGSK

SEQ ID NO: 63 - protein, 13 aa, synthetic construct
HEVQEHFPEALAR

SEQ ID NO: 64 - protein, 8 aa, synthetic construct
NANLSTIK

SEQ ID NO: 65 - protein, 9 aa, synthetic construct
EIFPQSLPK

SEQ ID NO: 66 - protein, 15 aa, synthetic construct
AAEIASSDSANVSSR

SEQ ID NO: 67 - protein, 8 aa, synthetic construct
EATISYIK

SEQ ID NO: 68 - protein, 12 aa, synthetic construct
VFGEDSVGVIFK

SEQ ID NO: 69 - protein, 10 aa, synthetic construct
EYNSGDDLDR

SEQ ID NO: 70 - protein, 7 aa, synthetic construct
VLQGLPR

SEQ ID NO: 71 - protein, 15 aa, synthetic construct
GLQSLPTHDPSPLQR

SEQ ID NO: 72 - protein, 21 aa, synthetic construct
FVVIQNEDLGPASPLDSTFYR

SEQ ID NO: 73 - protein, 13 aa, synthetic construct
TDLHAFENLEIIR

SEQ ID NO: 74 - protein, 10 aa, synthetic construct
IPLENLQIIR

SEQ ID NO: 75 - protein, 9 aa, synthetic construct
YLVIQGDER

SEQ ID NO: 76 - protein, 14 aa, synthetic construct
LVINGNPITIFQER

SEQ ID NO: 77 - protein, 15 aa, synthetic construct
GALQNIIPASTGAAK

SEQ ID NO: 78 - protein, 8 aa, synthetic construct
VGVNGFGR

SEQ ID NO: 79 - protein, 10 aa, synthetic construct
AGFAGDDAPR

SEQ ID NO: 80 - protein, 11 aa, synthetic construct
AVFPSIVGRPR

SEQ ID NO: 81 - protein, 16 aa, synthetic construct
SYELPDGQVITIGNER

SEQ ID NO: 82 - protein, 12 aa, synthetic construct
QVTQEEGQQLAR

SEQ ID NO: 83 - protein, 8 aa, synthetic construct
GSFEEIYK

SEQ ID NO: 84 - protein, 11 aa, synthetic construct
LVVVGGGGVGK

SEQ ID NO: 85 - protein, 18 aa, synthetic construct
VQAEDLLYGKPDGAFLIR

SEQ ID NO: 86 - protein, 9 aa, synthetic construct
HCVIYSTAR

SEQ ID NO: 87 - protein, 11 aa, synthetic construct
LQEYHSQYQEK

SEQ ID NO: 88 - protein, 4 aa, synthetic construct
MAGR

SEQ ID NO: 89 - protein, 12 aa, synthetic construct
LAAALEHHHHHH

SEQ ID NO: 90 - protein, 8 aa, synthetic construct
DYKDDDDK

SEQ ID NO: 91 - protein, 9 aa, synthetic construct
YPYDVPDYA

SEQ ID NO: 92 - protein, 14 aa, synthetic construct
GKPIPNPLLGLDST

SEQ ID NO: 93 - protein, 189 aa, synthetic construct
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 94 - protein, 189 aa, synthetic construct [sequence omitted]

SEQ ID NO: 95 - protein, 189 aa, synthetic construct
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 96 - protein, 180 aa, synthetic construct
IGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAF

SEQ ID NO: 97 - protein, 180 aa, synthetic construct
IGTGSFGTVFRGRWHGDVAVKVLKVSQPTAEQAQAFKNEMQVLRKTRHVNILLFMGFMTRPGFAIITQWCEGSSLYHHLHVADTRFDMVQLIDVARQTAQGMDYLHAKNIIHRDLKSNNIFLHEGLTVKIGDFGLATVKTRWSGAQPLEQPSGSVLWMAAEVIRMQDPNPYSFQSDVYAY

SEQ ID NO: 98 - protein, 180 aa, synthetic construct
IGSGSFGTVYKGKWHGDVAVKILKVVDPTPEQFQAFRNEVAVLRKTRHVNILLFMGYMTKDNLAIVTQWCEGSSLYKHLHVQETKFQMFQLIDIARQTAQGMDYLHAKNIIHRDMKSNNIFLHEGLTVKIGDFGLATVKSRWSGSQQVEQPTGSVLWMAPEVIRMQDNNPFSFQSDVYSY

SEQ ID NO: 99 - protein, 303 aa, synthetic construct
MEHIQGAWKTISNGFGFKDAVFDGSSCISPTIVQQFGYQRRASDDGKLTDPSKTSNTIRVFLPNKQRTVVNVRNGMSLHDCLMKALKVRGLQPECCAVFRLLHEHKGKKARLDWNTDAASLIGEELQVDFLDHVPLTTHNFARKTFLKLAFCDICQKFLLNGFRCQTCGYKFHEHCSTKVPTMCVDWSNIRQLLLFPNSTIGDSGVPALPSLTMRRMRESVSRMPVSSQHRYSTPHAFTFNTSSPSSEGSLSQRQRSTSTPNVHMVSTTLPVDSRMIEDAIRSHSESASPSALSSSPNNLSPT

SEQ ID NO: 100 - protein, 346 aa, synthetic construct
TGWSQPKTPVPAQRERAPVSGTQEKNKIRPRGQRDSSYYWEIEASEVMLSTRIGSGSFGTVYKGKWHGDVAVKILKVVDPTPEQFQAFRNEVAVLRKTRHVNILLFMGYMTKDNLAIVTQWCEGSSLYKHLHVQETKFQMFQLIDIARQTAQGMDYLHAKNIIHRDMKSNNIFLHEGLTVKIGDFGLATVKSRWSGSQQVEQPTGSVLWMAPEVIRMQDNNPFSFQSDVYSYGIVLYELMTGELPYSHINNRDQIIFMVGRGYASPDLSKLYKNCPKAMKRLVADCVKKVKEERPLFPQILSSIELLQHSLPKINRSASEPSLHRAAHTEDINACTLTTSPRLPVF

SEQ ID NO: 101 - protein, 346 aa, synthetic construct
TGWSQPKTPVPAQRERAPVSGTQEKNKIRPRGQRDSSYYWEIEASEVMLSTRIGSGSFGTVYKGKWHGDVAVKILKVVDPTPEQFQAFRNEVAVLRKTRHVNILLFMGYMTKDNLAIVTQWCEGSSLYKHLHVQETKFQMFQLIDIARQTAQGMDYLHAKNIIHRNMKSNNIFLHEGLTVKIGDFGLATVKSRWSGSQQVEQPTGSVLWMAPEVIRMQDNNPFSFQSDVYSYGIVLYELMTGELPYSHINNRDQIIFMVGRGYASPDLSKLYKNCPKAMKRLVADCVKKVKEERPLFPQILSSIELLQHSLPKINRSASEPSLHRAAHTEDINACTLTTSPRLPVF

SEQ ID NO: 102 - protein, 346 aa, synthetic construct
TGWSQPKTPVPAQRERAPVSGTQEKNKIRPRGQRDSSYYWEIEASEVMLSTRIGSGSFGTVYKGKWHGDVAVMILKVVDPTPEQFQAFRNEVAVLRKTRHVNILLFMGYMTKDNLAIVTQWCEGSSLYKHLHVQETKFQMFQLIDIARQTAQGMDYLHAKNIIHRDMKSNNIFLHEGLTVKIGDFGLATVKSRWSGSQQVEQPTGSVLWMAPEVIRMQDNNPFSFQSDVYSYGIVLYELMTGELPYSHINNRDQIIFMVGRGYASPDLSKLYKNCPKAMKRLVADCVKKVKEERPLFPQILSSIELLQHSLPKINRSASEPSLHRAAHTEDINACTLTTSPRLPVF